SEQUENCE LISTING (24 sequences)

SEQ ID NO: 1 - protein, 841 aa, Artificial Sequence
  Synthetically generated
GVMVHLFQWKYNDIANECEKVLGPKGYEAVQITPPAEHLQGSSWWVVYQPVSYKNFTSLGGNEAELKSMIARCKAAGVKIYADAVFNQLAGGSGVGTGGSSYNAGSFSYPQFGYNDFHHAGSLTNYADRNNVQNGALLGLPDLDTGSAYVQDQLATYMKTLSGWGVAGFRLDAAKHMSVADLSAIVSKAGNPFVYSEVIGATGEPIQPGEYTGIGAVTEFKYGTDLASNFKGQIKNLKSMGESWGLLASNKAEVFVVNHDRERGHGGGGMLTYKDGALYNLANIFMLAWPYGAYPQVMSGYDFGTNTDIGGPSATPCSSGSSWNCEHRWSNIANMVSFHNAAQGTSMTNWWDNGNNQIAFGRGAKAFVVINNESSTLSKSLQTGLPAGEYCNILAGDALCSGSTIKVDASGMATFNVAGMKAAAIHINAKPDSTSSGSSGSSSGSSSSATSNKFASMNLRGTNNGWASTAMTVDANRVWSADVTFTGAADANGAQRFKFDVYGNWTESYGDTQADGIADKGSAKDIYFNGVGKYRVSLKESDMSYTLTQLSSNQAPVAAITPKTLSVKLGDSVVFDASGSTDDVGVTGYSWSTGGSAKTETVLFDALGTKTITVTVADADGLTSKASATVTVTDGSVAYNSNFASLNFRGTPNSWGAAAMTLVADNTWEATVNFDGQANQRFKFDIKGDWSQNYGDSNKDGVAERTGADIYTTVTGQYKVQFNDSTLKYTLTKLADSSATSYSANFASLYLRGTPNSWGTTAMKLVANNSWQAEVTFTGKGDATGAQRFKFDVKGDWSQNYGDSNMDGTAERTGGDITSAVVGTYLVTFNDSTLKYTLTAK

SEQ ID NO: 2 - protein, 429 aa, Artificial Sequence
  Synthetically generated
GVMVHLFQWKYNDIANECEKVLGPKGYEAVQITPPAEHLQGSSWWVVYQPVSYKNFTSLGGNEAELKSMIARCKAAGVKIYADAVFNQLAGGSGVGTGGSSYNAGSFSYPQFGYNDFHHAGSLTNYADRNNVQNGALLGLPDLDTGSAYVQDQLATYMKTLSGWGVAGFRLDAAKHMSVADLSAIVSKAGNPFVYSEVIGATGEPIQPGEYTGIGAVTEFKYGTDLASNFKGQIKNLKSMGESWGLLASNKAEVFVVNHDRERGHGGGGMLTYKDGALYNLANIFMLAWPYGAYPQVMSGYDFGTNTDIGGPSATPCSSGSSWNCEHRWSNIANMVSFHNAAQGTSMTNWWDNGNNQIAFGRGAKAFVVINNESSTLSKSLQTGLPAGEYCNILAGDALCSGSTIKVDASGMATFNVAGMKAAAIHINA

SEQ ID NO: 3 - protein, 430 aa, Artificial Sequence
  Synthetically generated
GVMVHLFQWKYNDIANECEKVLGPKGYEAVQITPPAEHLQGSSWWVVYQPVSYKNFTSLGGNEAELKSMIARCKAAGVKIYADAVFNQLAGGSGVGTGGSSYNAGSFSYPQFGYNDFHHAGSLTNYADRNNVQNGALLGLPDLDTGSAYVQDQLATYMKTLSGWGVAGFRLDAAKHMSVADLSAIVSKAGNPFVYSEVIGATGEPIQPGEYTGIGAVTEFKYGTDLASNFKGQIKNLKSMGESWGLLASNKAEVFVVNHDRERGHGGGGMLTYKDGALYNLANIFMLAWPYGAYPQVMSGYDFGTNTDIGGPSATPCSSGSSWNCEHRWSNIANMVSFHNAAQGTSMTNWWDNGNNQIAFGRGAKAFVVINNESSTLSKSLQTGLPAGEYCNILAGDALCSGSTIKVDASGMATFNVAGMKAAAIHINAK

SEQ ID NO: 4 - protein, 431 aa, Artificial Sequence
  Synthetically generated
GVMVHLFQWKYNDIANECEKVLGPKGYEAVQITPPAEHLQGSSWWVVYQPVSYKNFTSLGGNEAELKSMIARCKAAGVKIYADAVFNQLAGGSGVGTGGSSYNAGSFSYPQFGYNDFHHAGSLTNYADRNNVQNGALLGLPDLDTGSAYVQDQLATYMKTLSGWGVAGFRLDAAKHMSVADLSAIVSKAGNPFVYSEVIGATGEPIQPGEYTGIGAVTEFKYGTDLASNFKGQIKNLKSMGESWGLLASNKAEVFVVNHDRERGHGGGGMLTYKDGALYNLANIFMLAWPYGAYPQVMSGYDFGTNTDIGGPSATPCSSGSSWNCEHRWSNIANMVSFHNAAQGTSMTNWWDNGNNQIAFGRGAKAFVVINNESSTLSKSLQTGLPAGEYCNILAGDALCSGSTIKVDASGMATFNVAGMKAAAIHINAKP

SEQ ID NO: 5 - protein, 432 aa, Artificial Sequence
  Synthetically generated
GVMVHLFQWKYNDIANECEKVLGPKGYEAVQITPPAEHLQGSSWWVVYQPVSYKNFTSLGGNEAELKSMIARCKAAGVKIYADAVFNQLAGGSGVGTGGSSYNAGSFSYPQFGYNDFHHAGSLTNYADRNNVQNGALLGLPDLDTGSAYVQDQLATYMKTLSGWGVAGFRLDAAKHMSVADLSAIVSKAGNPFVYSEVIGATGEPIQPGEYTGIGAVTEFKYGTDLASNFKGQIKNLKSMGESWGLLASNKAEVFVVNHDRERGHGGGGMLTYKDGALYNLANIFMLAWPYGAYPQVMSGYDFGTNTDIGGPSATPCSSGSSWNCEHRWSNIANMVSFHNAAQGTSMTNWWDNGNNQIAFGRGAKAFVVINNESSTLSKSLQTGLPAGEYCNILAGDALCSGSTIKVDASGMATFNVAGMKAAAIHINAKPD

SEQ ID NO: 6 - protein, 433 aa, Artificial Sequence
  Synthetically generated
GVMVHLFQWKYNDIANECEKVLGPKGYEAVQITPPAEHLQGSSWWVVYQPVSYKNFTSLGGNEAELKSMIARCKAAGVKIYADAVFNQLAGGSGVGTGGSSYNAGSFSYPQFGYNDFHHAGSLTNYADRNNVQNGALLGLPDLDTGSAYVQDQLATYMKTLSGWGVAGFRLDAAKHMSVADLSAIVSKAGNPFVYSEVIGATGEPIQPGEYTGIGAVTEFKYGTDLASNFKGQIKNLKSMGESWGLLASNKAEVFVVNHDRERGHGGGGMLTYKDGALYNLANIFMLAWPYGAYPQVMSGYDFGTNTDIGGPSATPCSSGSSWNCEHRWSNIANMVSFHNAAQGTSMTNWWDNGNNQIAFGRGAKAFVVINNESSTLSKSLQTGLPAGEYCNILAGDALCSGSTIKVDASGMATFNVAGMKAAAIHINAKPDS

SEQ ID NO: 7 - protein, 434 aa, Artificial Sequence
  Synthetically generated
GVMVHLFQWKYNDIANECEKVLGPKGYEAVQITPPAEHLQGSSWWVVYQPVSYKNFTSLGGNEAELKSMIARCKAAGVKIYADAVFNQLAGGSGVGTGGSSYNAGSFSYPQFGYNDFHHAGSLTNYADRNNVQNGALLGLPDLDTGSAYVQDQLATYMKTLSGWGVAGFRLDAAKHMSVADLSAIVSKAGNPFVYSEVIGATGEPIQPGEYTGIGAVTEFKYGTDLASNFKGQIKNLKSMGESWGLLASNKAEVFVVNHDRERGHGGGGMLTYKDGALYNLANIFMLAWPYGAYPQVMSGYDFGTNTDIGGPSATPCSSGSSWNCEHRWSNIANMVSFHNAAQGTSMTNWWDNGNNQIAFGRGAKAFVVINNESSTLSKSLQTGLPAGEYCNILAGDALCSGSTIKVDASGMATFNVAGMKAAAIHINAKPDST

SEQ ID NO: 8 - protein, 437 aa, Artificial Sequence
  Synthetically generated
GVMVHLFQWKYNDIANECEKVLGPKGYEAVQITPPAEHLQGSSWWVVYQPVSYKNFTSLGGNEAELKSMIARCKAAGVKIYADAVFNQLAGGSGVGTGGSSYNAGSFSYPQFGYNDFHHAGSLTNYADRNNVQNGALLGLPDLDTGSAYVQDQLATYMKTLSGWGVAGFRLDAAKHMSVADLSAIVSKAGNPFVYSEVIGATGEPIQPGEYTGIGAVTEFKYGTDLASNFKGQIKNLKSMGESWGLLASNKAEVFVVNHDRERGHGGGGMLTYKDGALYNLANIFMLAWPYGAYPQVMSGYDFGTNTDIGGPSATPCSSGSSWNCEHRWSNIANMVSFHNAAQGTSMTNWWDNGNNQIAFGRGAKAFVVINNESSTLSKSLQTGLPAGEYCNILAGDALCSGSTIKVDASGMATFNVAGMKAAAIHINAKPDSTSSG

SEQ ID NO: 9 - protein, 440 aa, Artificial Sequence
  Synthetically generated
GVMVHLFQWKYNDIANECEKVLGPKGYEAVQITPPAEHLQGSSWWVVYQPVSYKNFTSLGGNEAELKSMIARCKAAGVKIYADAVFNQLAGGSGVGTGGSSYNAGSFSYPQFGYNDFHHAGSLTNYADRNNVQNGALLGLPDLDTGSAYVQDQLATYMKTLSGWGVAGFRLDAAKHMSVADLSAIVSKAGNPFVYSEVIGATGEPIQPGEYTGIGAVTEFKYGTDLASNFKGQIKNLKSMGESWGLLASNKAEVFVVNHDRERGHGGGGMLTYKDGALYNLANIFMLAWPYGAYPQVMSGYDFGTNTDIGGPSATPCSSGSSWNCEHRWSNIANMVSFHNAAQGTSMTNWWDNGNNQIAFGRGAKAFVVINNESSTLSKSLQTGLPAGEYCNILAGDALCSGSTIKVDASGMATFNVAGMKAAAIHINAKPDSTSSGSSG

SEQ ID NO: 10 - protein, 433 aa, Artificial Sequence
  Synthetically generated
GVMVHLFQWKYNDIANECEKVLGPKGYEAVQITPPAEHLQGSSWWVVYQPVSYKNFTSLGGNEAELKSMIARCKAAGVKIYADAVFNQLAGGSGVGTGGSSYNAGSFSYPQFGYNDFHHAGPLTNYTDRNNVQNGALHGLPDLDTGSAYVQDQLATYMKTLSGWGVAGFRLDAAKHMSVADLSAIVSKAGNPFVYSEVIGATGEPIQPGEYTGIGAVTEFKYGTDLASNFKGQIKNLKSMGESWGLLASNKAEVFVVNHDRERGHGGGGMLTYKDGALYNLANIFMLAWPYGAYPQVMSGYDFGTNTDIGGPSATPCSSGSSWNCEHRWSNIANMVSFHNAAQGTSMTNWWDNGNNQIAFGRGAKAFVVINNESSTLRKKLQTGLPAGEYCNILAGDALCSGSTIKVDASGMATFNVAGMKAAAIHINAKPDS

SEQ ID NO: 11 - protein, 23 aa, Artificial Sequence
  Synthetically generated
MKSRAWLLGSAVAMALASSAANA

SEQ ID NO: 12 - protein, 85 aa, Artificial Sequence
  Synthetically generated
MRFPSIFTAVLFAASSALAAPVNTTTEDETAQIPAEAVIGYSDLEGDFDVAVLPFSNSTNNGLLFINTTIASIAAKEEGVSLEKR

SEQ ID NO: 13 - DNA, 2526 bp, Artificial Sequence
  Synthetically generated
ggtgtcatggttcacctgttccagtggaagtacaatgacatcgccaacgagtgcgaaaaggtgctcggtcccaaagggtatgaagcagtgcagatcacgccgcctgctgaacacctgcaaggctcctcctggtgggtggtctatcagcccgtcagctacaagaacttcacttctctgggcggtaacgaggccgaactcaaaagcatgatcgcccgttgcaaggccgccggggtcaagatttacgccgatgcggtattcaaccagctggctggtggatcaggcgtcggtacaggtggtagcagctacaatgccggcagcttcagctatccccaatttggctacaacgatttccatcacgctgggagcctcaccaactatgccgaccgcaacaatgtgcaaaacggtgccctgctggggctgccggatctggataccggctctgcctatgtgcaggatcagctggctacctatatgaagaccctgagtggctggggtgtggcaggttttcgtcttgatgcagcaaagcatatgagcgttgccgatctctcggccatcgtcagcaaggcgggcaatccttttgtctactccgaggtgattggtgccacgggtgaaccaatccagccgggcgaatataccggcattggtgccgtgaccgaatttaaatacggcaccgatctggcctccaacttcaaggggcagatcaagaatctcaagagcatgggcgagagctggggtctgcttgcgtcgaacaaggctgaagtctttgtggtcaaccatgaccgtgagcggggacatggcggtggcggtatgctgacctacaaggatggtgccctctacaatctggccaacatcttcatgctggcctggccctatggcgcctatccccaggtgatgtccggctatgatttcggcaccaataccgatattggtgggccgagcgctaccccttgttcttccggctctagctggaactgcgaacaccgctggagcaacatcgccaacatggtctcgttccacaatgccgcccaaggcacgtccatgaccaactggtgggataatggtaataaccagatcgcctttggtcgcggcgccaaggcctttgtggtgatcaacaatgaatcttccactctgagcaagagcctgcagacgggtctgccagccggggagtactgcaacattctggccggtgatgccctgtgcagcggcagcaccatcaaggtggatgccagcggtatggccaccttcaacgtggcagggatgaaggcggcagcgatccatatcaatgccaagcccgatagcaccagcagtggcagctcaggctcttcctctggctcttcttcctctgccaccagtaacaagtttgccagcatgaatctgcggggcaccaacaatggctgggccagcaccgccatgacagtggatgccaaccgtgtctggtcggcggatgtcacctttaccggggccgcggatgccaatggtgcccagcgcttcaagtttgatgtctatggcaactggacagagagctatggcgatacacaagccgatggcattgccgacaaggggagcgccaaggacatctatttcaatggtgtgggcaagtatcgtgtctcgctcaaggagagcgacatgagctacaccctgacccagctctccagcaatcaggcaccggtggcggccatcacccccaagacactctccgtcaagctgggtgactcagtggtgttcgatgcctccggctccaccgatgatgtgggtgtcactggctacagctggtctaccggtggcagtgccaagaccgaaactgtgctgtttgatgctctgggtaccaagaccattaccgtgacagtggccgatgccgatggcttgacctccaaggccagtgccaccgtcaccgtcaccgatggcagcgtggcttataacagcaactttgccagcctgaacttccgtggcactcccaacagttggggcgcggcagccatgacgctggtggcagacaacacctgggaggcaacggtcaacttcgatggtcaggccaatcagcgcttcaagttcgatatcaagggtgactggagccagaactatggtgatagcaacaaggatggggtggccgaacgtaccggtgccgatatttacaccactgtgaccggtcaatataaggtgcaatttaacgactccactttgaagtacaccctgaccaagctggccgatagcagcgccaccagctatagcgcgaactttgccagcctctacctgcgtggcaccccgaacagctggggcaccaccgccatgaagctggtggccaataacagctggcaggccgaggtgaccttcaccggcaagggcgatgccactggtgcccaacgcttcaagttcgacgtcaagggtgactggagccagaactacggtgacagcaacatggacgggactgccgaacggactggtggcgatatcaccagtgccgtggtgggcacctatctggtgacctttaatgacagcacactgaaatacaccctgaccgccaaataa

SEQ ID NO: 14 - DNA, 1290 bp, Artificial Sequence
  Synthetically generated
ggtgtcatggttcacctgttccagtggaagtacaatgacatcgccaacgagtgcgaaaaggtgctcggtcccaaagggtatgaagcagtgcagatcacgccgcctgctgaacacctgcaaggctcctcctggtgggtggtctatcagcccgtcagctacaagaacttcacttctctgggcggtaacgaggccgaactcaaaagcatgatcgcccgttgcaaggccgccggggtcaagatttacgccgatgcggtattcaaccagctggctggtggatcaggcgtcggtacaggtggtagcagctacaatgccggcagcttcagctatccccaatttggctacaacgatttccatcacgctgggagcctcaccaactatgccgaccgcaacaatgtgcaaaacggtgccctgctggggctgccggatctggataccggctctgcctatgtgcaggatcagctggctacctatatgaagaccctgagtggctggggtgtggcaggttttcgtcttgatgcagcaaagcatatgagcgttgccgatctctcggccatcgtcagcaaggcgggcaatccttttgtctactccgaggtgattggtgccacgggtgaaccaatccagccgggcgaatataccggcattggtgccgtgaccgaatttaaatacggcaccgatctggcctccaacttcaaggggcagatcaagaatctcaagagcatgggcgagagctggggtctgcttgcgtcgaacaaggctgaagtctttgtggtcaaccatgaccgtgagcggggacatggcggtggcggtatgctgacctacaaggatggtgccctctacaatctggccaacatcttcatgctggcctggccctatggcgcctatccccaggtgatgtccggctatgatttcggcaccaataccgatattggtgggccgagcgctaccccttgttcttccggctctagctggaactgcgaacaccgctggagcaacatcgccaacatggtctcgttccacaatgccgcccaaggcacgtccatgaccaactggtgggataatggtaataaccagatcgcctttggtcgcggcgccaaggcctttgtggtgatcaacaatgaatcttccactctgagcaagagcctgcagacgggtctgccagccggggagtactgcaacattctggccggtgatgccctgtgcagcggcagcaccatcaaggtggatgccagcggtatggccaccttcaacgtggcagggatgaaggcggcagcgatccatatcaatgcctaa

SEQ ID NO: 15 - DNA, 1293 bp, Artificial Sequence
  Synthetically generated
ggtgtcatggttcacctgttccagtggaagtacaatgacatcgccaacgagtgcgaaaaggtgctcggtcccaaagggtatgaagcagtgcagatcacgccgcctgctgaacacctgcaaggctcctcctggtgggtggtctatcagcccgtcagctacaagaacttcacttctctgggcggtaacgaggccgaactcaaaagcatgatcgcccgttgcaaggccgccggggtcaagatttacgccgatgcggtattcaaccagctggctggtggatcaggcgtcggtacaggtggtagcagctacaatgccggcagcttcagctatccccaatttggctacaacgatttccatcacgctgggagcctcaccaactatgccgaccgcaacaatgtgcaaaacggtgccctgctggggctgccggatctggataccggctctgcctatgtgcaggatcagctggctacctatatgaagaccctgagtggctggggtgtggcaggttttcgtcttgatgcagcaaagcatatgagcgttgccgatctctcggccatcgtcagcaaggcgggcaatccttttgtctactccgaggtgattggtgccacgggtgaaccaatccagccgggcgaatataccggcattggtgccgtgaccgaatttaaatacggcaccgatctggcctccaacttcaaggggcagatcaagaatctcaagagcatgggcgagagctggggtctgcttgcgtcgaacaaggctgaagtctttgtggtcaaccatgaccgtgagcggggacatggcggtggcggtatgctgacctacaaggatggtgccctctacaatctggccaacatcttcatgctggcctggccctatggcgcctatccccaggtgatgtccggctatgatttcggcaccaataccgatattggtgggccgagcgctaccccttgttcttccggctctagctggaactgcgaacaccgctggagcaacatcgccaacatggtctcgttccacaatgccgcccaaggcacgtccatgaccaactggtgggataatggtaataaccagatcgcctttggtcgcggcgccaaggcctttgtggtgatcaacaatgaatcttccactctgagcaagagcctgcagacgggtctgccagccggggagtactgcaacattctggccggtgatgccctgtgcagcggcagcaccatcaaggtggatgccagcggtatggccaccttcaacgtggcagggatgaaggcggcagcgatccatatcaatgccaagtaa

SEQ ID NO: 16 - DNA, 1296 bp, Artificial Sequence
  Synthetically generated
ggtgtcatggttcacctgttccagtggaagtacaatgacatcgccaacgagtgcgaaaaggtgctcggtcccaaagggtatgaagcagtgcagatcacgccgcctgctgaacacctgcaaggctcctcctggtgggtggtctatcagcccgtcagctacaagaacttcacttctctgggcggtaacgaggccgaactcaaaagcatgatcgcccgttgcaaggccgccggggtcaagatttacgccgatgcggtattcaaccagctggctggtggatcaggcgtcggtacaggtggtagcagctacaatgccggcagcttcagctatccccaatttggctacaacgatttccatcacgctgggagcctcaccaactatgccgaccgcaacaatgtgcaaaacggtgccctgctggggctgccggatctggataccggctctgcctatgtgcaggatcagctggctacctatatgaagaccctgagtggctggggtgtggcaggttttcgtcttgatgcagcaaagcatatgagcgttgccgatctctcggccatcgtcagcaaggcgggcaatccttttgtctactccgaggtgattggtgccacgggtgaaccaatccagccgggcgaatataccggcattggtgccgtgaccgaatttaaatacggcaccgatctggcctccaacttcaaggggcagatcaagaatctcaagagcatgggcgagagctggggtctgcttgcgtcgaacaaggctgaagtctttgtggtcaaccatgaccgtgagcggggacatggcggtggcggtatgctgacctacaaggatggtgccctctacaatctggccaacatcttcatgctggcctggccctatggcgcctatccccaggtgatgtccggctatgatttcggcaccaataccgatattggtgggccgagcgctaccccttgttcttccggctctagctggaactgcgaacaccgctggagcaacatcgccaacatggtctcgttccacaatgccgcccaaggcacgtccatgaccaactggtgggataatggtaataaccagatcgcctttggtcgcggcgccaaggcctttgtggtgatcaacaatgaatcttccactctgagcaagagcctgcagacgggtctgccagccggggagtactgcaacattctggccggtgatgccctgtgcagcggcagcaccatcaaggtggatgccagcggtatggccaccttcaacgtggcagggatgaaggcggcagcgatccatatcaatgccaagccctaa

SEQ ID NO: 17 - DNA, 1299 bp, Artificial Sequence
  Synthetically generated
ggtgtcatggttcacctgttccagtggaagtacaatgacatcgccaacgagtgcgaaaaggtgctcggtcccaaagggtatgaagcagtgcagatcacgccgcctgctgaacacctgcaaggctcctcctggtgggtggtctatcagcccgtcagctacaagaacttcacttctctgggcggtaacgaggccgaactcaaaagcatgatcgcccgttgcaaggccgccggggtcaagatttacgccgatgcggtattcaaccagctggctggtggatcaggcgtcggtacaggtggtagcagctacaatgccggcagcttcagctatccccaatttggctacaacgatttccatcacgctgggagcctcaccaactatgccgaccgcaacaatgtgcaaaacggtgccctgctggggctgccggatctggataccggctctgcctatgtgcaggatcagctggctacctatatgaagaccctgagtggctggggtgtggcaggttttcgtcttgatgcagcaaagcatatgagcgttgccgatctctcggccatcgtcagcaaggcgggcaatccttttgtctactccgaggtgattggtgccacgggtgaaccaatccagccgggcgaatataccggcattggtgccgtgaccgaatttaaatacggcaccgatctggcctccaacttcaaggggcagatcaagaatctcaagagcatgggcgagagctggggtctgcttgcgtcgaacaaggctgaagtctttgtggtcaaccatgaccgtgagcggggacatggcggtggcggtatgctgacctacaaggatggtgccctctacaatctggccaacatcttcatgctggcctggccctatggcgcctatccccaggtgatgtccggctatgatttcggcaccaataccgatattggtgggccgagcgctaccccttgttcttccggctctagctggaactgcgaacaccgctggagcaacatcgccaacatggtctcgttccacaatgccgcccaaggcacgtccatgaccaactggtgggataatggtaataaccagatcgcctttggtcgcggcgccaaggcctttgtggtgatcaacaatgaatcttccactctgagcaagagcctgcagacgggtctgccagccggggagtactgcaacattctggccggtgatgccctgtgcagcggcagcaccatcaaggtggatgccagcggtatggccaccttcaacgtggcagggatgaaggcggcagcgatccatatcaatgccaagcccgattaa

SEQ ID NO: 18 - DNA, 1302 bp, Artificial Sequence
  Synthetically generated
ggtgtcatggttcacctgttccagtggaagtacaatgacatcgccaacgagtgcgaaaaggtgctcggtcccaaagggtatgaagcagtgcagatcacgccgcctgctgaacacctgcaaggctcctcctggtgggtggtctatcagcccgtcagctacaagaacttcacttctctgggcggtaacgaggccgaactcaaaagcatgatcgcccgttgcaaggccgccggggtcaagatttacgccgatgcggtattcaaccagctggctggtggatcaggcgtcggtacaggtggtagcagctacaatgccggcagcttcagctatccccaatttggctacaacgatttccatcacgctgggagcctcaccaactatgccgaccgcaacaatgtgcaaaacggtgccctgctggggctgccggatctggataccggctctgcctatgtgcaggatcagctggctacctatatgaagaccctgagtggctggggtgtggcaggttttcgtcttgatgcagcaaagcatatgagcgttgccgatctctcggccatcgtcagcaaggcgggcaatccttttgtctactccgaggtgattggtgccacgggtgaaccaatccagccgggcgaatataccggcattggtgccgtgaccgaatttaaatacggcaccgatctggcctccaacttcaaggggcagatcaagaatctcaagagcatgggcgagagctggggtctgcttgcgtcgaacaaggctgaagtctttgtggtcaaccatgaccgtgagcggggacatggcggtggcggtatgctgacctacaaggatggtgccctctacaatctggccaacatcttcatgctggcctggccctatggcgcctatccccaggtgatgtccggctatgatttcggcaccaataccgatattggtgggccgagcgctaccccttgttcttccggctctagctggaactgcgaacaccgctggagcaacatcgccaacatggtctcgttccacaatgccgcccaaggcacgtccatgaccaactggtgggataatggtaataaccagatcgcctttggtcgcggcgccaaggcctttgtggtgatcaacaatgaatcttccactctgagcaagagcctgcagacgggtctgccagccggggagtactgcaacattctggccggtgatgccctgtgcagcggcagcaccatcaaggtggatgccagcggtatggccaccttcaacgtggcagggatgaaggcggcagcgatccatatcaatgccaagcccgatagctaa

SEQ ID NO: 19 - DNA, 1305 bp, Artificial Sequence
  Synthetically generated
ggtgtcatggttcacctgttccagtggaagtacaatgacatcgccaacgagtgcgaaaaggtgctcggtcccaaagggtatgaagcagtgcagatcacgccgcctgctgaacacctgcaaggctcctcctggtgggtggtctatcagcccgtcagctacaagaacttcacttctctgggcggtaacgaggccgaactcaaaagcatgatcgcccgttgcaaggccgccggggtcaagatttacgccgatgcggtattcaaccagctggctggtggatcaggcgtcggtacaggtggtagcagctacaatgccggcagcttcagctatccccaatttggctacaacgatttccatcacgctgggagcctcaccaactatgccgaccgcaacaatgtgcaaaacggtgccctgctggggctgccggatctggataccggctctgcctatgtgcaggatcagctggctacctatatgaagaccctgagtggctggggtgtggcaggttttcgtcttgatgcagcaaagcatatgagcgttgccgatctctcggccatcgtcagcaaggcgggcaatccttttgtctactccgaggtgattggtgccacgggtgaaccaatccagccgggcgaatataccggcattggtgccgtgaccgaatttaaatacggcaccgatctggcctccaacttcaaggggcagatcaagaatctcaagagcatgggcgagagctggggtctgcttgcgtcgaacaaggctgaagtctttgtggtcaaccatgaccgtgagcggggacatggcggtggcggtatgctgacctacaaggatggtgccctctacaatctggccaacatcttcatgctggcctggccctatggcgcctatccccaggtgatgtccggctatgatttcggcaccaataccgatattggtgggccgagcgctaccccttgttcttccggctctagctggaactgcgaacaccgctggagcaacatcgccaacatggtctcgttccacaatgccgcccaaggcacgtccatgaccaactggtgggataatggtaataaccagatcgcctttggtcgcggcgccaaggcctttgtggtgatcaacaatgaatcttccactctgagcaagagcctgcagacgggtctgccagccggggagtactgcaacattctggccggtgatgccctgtgcagcggcagcaccatcaaggtggatgccagcggtatggccaccttcaacgtggcagggatgaaggcggcagcgatccatatcaatgccaagcccgatagcacctaa

SEQ ID NO: 20 - DNA, 1314 bp, Artificial Sequence
  Synthetically generated
ggtgtcatggttcacctgttccagtggaagtacaatgacatcgccaacgagtgcgaaaaggtgctcggtcccaaagggtatgaagcagtgcagatcacgccgcctgctgaacacctgcaaggctcctcctggtgggtggtctatcagcccgtcagctacaagaacttcacttctctgggcggtaacgaggccgaactcaaaagcatgatcgcccgttgcaaggccgccggggtcaagatttacgccgatgcggtattcaaccagctggctggtggatcaggcgtcggtacaggtggtagcagctacaatgccggcagcttcagctatccccaatttggctacaacgatttccatcacgctgggagcctcaccaactatgccgaccgcaacaatgtgcaaaacggtgccctgctggggctgccggatctggataccggctctgcctatgtgcaggatcagctggctacctatatgaagaccctgagtggctggggtgtggcaggttttcgtcttgatgcagcaaagcatatgagcgttgccgatctctcggccatcgtcagcaaggcgggcaatccttttgtctactccgaggtgattggtgccacgggtgaaccaatccagccgggcgaatataccggcattggtgccgtgaccgaatttaaatacggcaccgatctggcctccaacttcaaggggcagatcaagaatctcaagagcatgggcgagagctggggtctgcttgcgtcgaacaaggctgaagtctttgtggtcaaccatgaccgtgagcggggacatggcggtggcggtatgctgacctacaaggatggtgccctctacaatctggccaacatcttcatgctggcctggccctatggcgcctatccccaggtgatgtccggctatgatttcggcaccaataccgatattggtgggccgagcgctaccccttgttcttccggctctagctggaactgcgaacaccgctggagcaacatcgccaacatggtctcgttccacaatgccgcccaaggcacgtccatgaccaactggtgggataatggtaataaccagatcgcctttggtcgcggcgccaaggcctttgtggtgatcaacaatgaatcttccactctgagcaagagcctgcagacgggtctgccagccggggagtactgcaacattctggccggtgatgccctgtgcagcggcagcaccatcaaggtggatgccagcggtatggccaccttcaacgtggcagggatgaaggcggcagcgatccatatcaatgccaagcccgatagcaccagcagtggctaa

SEQ ID NO: 21 - DNA, 1323 bp, Artificial Sequence
  Synthetically generated
ggtgtcatggttcacctgttccagtggaagtacaatgacatcgccaacgagtgcgaaaaggtgctcggtcccaaagggtatgaagcagtgcagatcacgccgcctgctgaacacctgcaaggctcctcctggtgggtggtctatcagcccgtcagctacaagaacttcacttctctgggcggtaacgaggccgaactcaaaagcatgatcgcccgttgcaaggccgccggggtcaagatttacgccgatgcggtattcaaccagctggctggtggatcaggcgtcggtacaggtggtagcagctacaatgccggcagcttcagctatccccaatttggctacaacgatttccatcacgctgggagcctcaccaactatgccgaccgcaacaatgtgcaaaacggtgccctgctggggctgccggatctggataccggctctgcctatgtgcaggatcagctggctacctatatgaagaccctgagtggctggggtgtggcaggttttcgtcttgatgcagcaaagcatatgagcgttgccgatctctcggccatcgtcagcaaggcgggcaatccttttgtctactccgaggtgattggtgccacgggtgaaccaatccagccgggcgaatataccggcattggtgccgtgaccgaatttaaatacggcaccgatctggcctccaacttcaaggggcagatcaagaatctcaagagcatgggcgagagctggggtctgcttgcgtcgaacaaggctgaagtctttgtggtcaaccatgaccgtgagcggggacatggcggtggcggtatgctgacctacaaggatggtgccctctacaatctggccaacatcttcatgctggcctggccctatggcgcctatccccaggtgatgtccggctatgatttcggcaccaataccgatattggtgggccgagcgctaccccttgttcttccggctctagctggaactgcgaacaccgctggagcaacatcgccaacatggtctcgttccacaatgccgcccaaggcacgtccatgaccaactggtgggataatggtaataaccagatcgcctttggtcgcggcgccaaggcctttgtggtgatcaacaatgaatcttccactctgagcaagagcctgcagacgggtctgccagccggggagtactgcaacattctggccggtgatgccctgtgcagcggcagcaccatcaaggtggatgccagcggtatggccaccttcaacgtggcagggatgaaggcggcagcgatccatatcaatgccaagcccgatagcaccagcagtggcagctcaggctaa

SEQ ID NO: 22 - DNA, 1302 bp, Artificial Sequence
  Synthetically generated
ggtgtcatggttcacctgttccagtggaagtacaatgacatcgccaacgagtgcgaaaaggtgctcggtcccaaagggtatgaagcagtgcagatcacgccgcctgctgaacacctgcaaggctcctcctggtgggtggtctatcagcccgtcagctacaagaacttcacttctctgggcggtaacgaggccgaactcaaaagcatgatcgcccgttgcaaggccgccggggtcaagatttacgccgatgcggtattcaaccagctggctggtggatcaggcgtcggtacaggtggtagcagctacaatgccggcagcttcagctatccccaatttggctacaacgatttccatcacgctgggcctctcaccaactatactgaccgcaacaatgtgcaaaacggtgccctgcacgggctgccggatctggataccggctctgcctatgtgcaggatcagctggctacctatatgaagaccctgagtggctggggtgtggcaggttttcgtcttgatgcagcaaagcatatgagcgttgccgatctctcggccatcgtcagcaaggcgggcaatccttttgtctactccgaggtgattggtgccacgggtgaaccaatccagccgggcgaatataccggcattggtgccgtgaccgaatttaaatacggcaccgatctggcctccaacttcaaggggcagatcaagaatctcaagagcatgggcgagagctggggtctgcttgcgtcgaacaaggctgaagtctttgtggtcaaccatgaccgtgagcggggacatggcggtggcggtatgctgacctacaaggatggtgccctctacaatctggccaacatcttcatgctggcctggccctatggcgcctatccccaggtgatgtccggctatgatttcggcaccaataccgatattggtgggccgagcgctaccccttgttcttccggctctagctggaactgcgaacaccgctggagcaacatcgccaacatggtctcgttccacaatgccgcccaaggcacgtccatgaccaactggtgggataatggtaataaccagatcgcctttggtcgcggcgccaaggcctttgtggtgatcaacaatgaatcttccactctgagaaagaagctgcagacgggtctgccagccggggagtactgcaacattctggccggtgatgccctgtgcagcggcagcaccatcaaggtggatgccagcggtatggccaccttcaacgtggcagggatgaaggcggcagcgatccatatcaatgccaagcccgatagctaa

SEQ ID NO: 23 - DNA, 69 bp, Artificial Sequence
  Synthetically generated
atgaagtcccgggcgtggttgttaggtagtgcagtggccatggcgttggcctcttcggcagccaatgcc

SEQ ID NO: 24 - DNA, 255 bp, Artificial Sequence
  Synthetically generated
atgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaaga

(19) United States
(12) Patent Application Publication
(10) Pub. No.: US 2021/0115422 A1
(43) Pub. Date: Apr. 22, 2021

(54) AMYLASE ENZYMES

(71) Applicant: BASF SE, Ludwigshafen (DE)

(72) Inventors: Asfia Qureshi, San Diego, CA (US); Adrienne Huston Davenport, San Diego, CA (US); Xuqiu Tan, San Diego, CA (US); Oliver Spangenberg, Ludwigshafen (DE); Amanda Rae Logue, San Diego, CA (US); Janina Berndt, Ludwigshafen (DE); Tong Li, San Diego, CA (US); Stefan Seemayer, Ludwigshafen (DE); Hans Wolfgang Hoeffken, Ludwigshafen (DE); Jingping Zhong, San Diego, CA (US)

(21) Appl. No.: 17/051,937

(22) PCT Filed: Apr. 24, 2019
(86) PCT No.: PCT/EP2019/060524

§ 371 Date: Oct. 30, 2020

Related U.S. Application Data
(60) Provisional application No. 62/666,253, filed on May 3, 2018.

(51) Int. Cl. C12N 9/28; A21D 8/04

(52) U.S. Cl. CPC C12N 9/2417; A23V 2002/00; A21D 8/042; C12Y 302/01001

(57) ABSTRACT

Engineered variant polypeptides having amylase enzyme activity, compositions comprising the enzymes, and methods of making and using the enzymes. The genetically engineered amylase enzymes are useful in many different applications such as laundry detergents, dish washing detergents, and cleaning products for homes, industry, vehicle care, baking, animal feed, pulp and paper processing, starch processing, and ethanol production.

### SEQUENCE LISTING

This application includes a nucleotide and amino acid sequence listing in computer readable form (CRF) as an ASC II text (.txt) file according to “Standard for the Presentation of Nucleotide and Amino Acid Sequence Listings in International Patent Applications Under the Patent Cooperation Treaty (PCT)” ST.25. The sequence listing is identified below and is hereby incorporated by reference into the specification of this application in its entirety and for all purposes.

| | Date of | |
|---|---|---|
| File Name | Creation | Size |
| 161162_SequenceListing_ST25 | Mar. 19, 2018 | 62.1 KB (63,678 bytes) |

### TECHNICAL FIELD

Genetically engineered amylase enzymes, compositions comprising the enzymes, and methods of using the enzymes or compositions comprising the enzymes. The genetically engineered amylase enzymes are useful in many different applications such as laundry detergents, dish washing detergents, and cleaning products for homes, industry, vehicle care, baking, animal feed, pulp and paper processing, starch processing, and ethanol production. Amylases have been employed in the removal of starch stains and have been added to various compositions such as cleaning products. Current cleaning and/or fabric care compositions comprise formulations of many active ingredients that impact with the ability of amylases to remove starch stains. Thus, the need exists for genetically engineered amylase enzymes that can function in the harsh environment of compositions used for cleaning.

### BRIEF DESCRIPTION OF THE SEVERAL VIEWS OF THE DRAWING

FIG. 1(A) to FIG. 1(H): shows the wash performance of the amylase of SEQ ID NO:1 (encoded by SEQ ID NO:13) and Stainzyme 12L on various starch stains (CS-26, CS-27, CS-28, CS-29 Center for Testmaterials B.V.).

FIG. 2(A) to FIG. 2(C): shows the wash performance of the amylase of SEQ ID NO:1 (encoded by SEQ ID NO:13) and Stainzyme 12L on cocoa oatmeal stain (KC-H097 ex CFT).

FIG. 3(A) to FIG. 3(E): shows the wash performance of the amylase of SEQ ID NO:1 (encoded by SEQ ID NO:13) and Stainzyme 12L on different fresh stains on knitted cotton from Warwick Equest Ltd.

FIG. 4: shows the wash performance of variants of the amylase variants and Stainzyme 12L, in a non-bio commercial liquid laundry formulation on 11 different amylase sensitive stains on knitted cotton on basis of ddE.

### DETAILED DESCRIPTION OF THE INVENTION

An enzyme is a biological molecule (polypeptide) comprising a sequence of amino acid residues, wherein the enzyme can catalyze a reaction. Hence, enzymes are catalytically active proteins or polypeptides. Enzyme names are determined based on the recommendations of the Nomenclature Committee of the International Union of Biochemistry and Molecular Biology (IUBMB). Enzymes are defined by an EC (Enzyme Commission) number, recommended name, alternative names (if any), catalytic activity, and other factors. Enzymes herein may be identified by polypeptide sequences (also called amino acid sequences herein). The polypeptide sequence specifies the three-dimensional structure including the “active site” of an enzyme which in turn determines the catalytic activity of the same. Polypeptide sequences may be identified by a SEQ ID NO.

Enzymes are obtained from or derived from many different sources including: plants; animals; bacteria, archea, fungi, yeast, environmental samples containing DNA that encodes an enzyme, or enzymes can be synthetic generated in a laboratory. For example, bacterial sources of enzymes include enzymes derived from Bacillus, Streptomyces, E. coli and Pseudomonas; fungal sources of enzymes include enzymes derived from Aspergillus, Fusarium, Thermomyces and Trichoderma; yeast sources of enzymes include enzymes derived from Pichia, and Saccharomyces.

Different classes of enzymes are known to be useful in detergents and cleaning products including: lipase, amylase, protease, cellulase, mannanase, pectate lyase, and nuclease; however, there is a need in the industry to provide an amylase that has more activity, temperature profile, pH profile, has improved performance (stain removal), stability in presence of protease, or a combination thereof. The variant polypeptide amylase enzymes address these industrial needs.

The World Intellectual Property Office (WIPO) Standard ST.25 (1998) provides that the amino acid residues should be represented in the sequence listing using the following three-letter symbols with the first letter as a capital. The table below provides an overview of the amino acid identifiers as well as the corresponding DNA codons that encode the amino acid using the standard genetic standard. The DNA codons that encode amino acid residues can be different depending organism that is used and slightly different tables for translation of the genetic code may apply. A compilation of such non-standard code translation tables is maintained at the NCBI. For reference see e.g. https://www.ncbi.nlm.nih.gov/Taxonomy/Utils/wprintgc.cgi.

| Amino Acids | | | Nucleic Acids |
|---|---|---|---|
| Name | 3 letter code | 1 letter code | DNA codons |
| Alanine | Ala | A | GCA, GCC, GCG, GCT |
| Arginine | Arg | R | AGA, AGG, CGA, CGC, |
| | | | CGG, CGT |
| Asparagine | Asn | N | AAC, AAT |
| Aspartic acid; | Asp | D | GAC, GAT |
| (Aspartate) | | | |
| Cysteine | Cys | C | TGC, TGT |
| Glutamic acid; | Glu | E | GAA, GAG |
| (Glutamate) | | | |
| Glutamine | Gln | Q | CAA, CAG |
| Glycine | Gly | G | GGA, GGC, GGG, GGT |
| Histidine | His | H | CAC, CAT |
| Isoleucine | Ile | I | ATA, ATC, ATT |
| Leucine | Leu | L | CTA, CTC, CTG, CTT, |
| | | | TTA, TTG |
| Lysine | Lys | K | AAA, AAG |
| Methionine | Met | M | ATG |
| Phenylalanine | Phe | F | TTC, TTT |
| Proline | Pro | P | CCA, CCC, CCG, CCT |
| Serine | Ser | S | AGC, AGT, TCA, TCC, |
| | | | TCG, TCT |
| Threonine | Thr | T | ACA, ACC, ACG, ACT |
| Tryptophan | Trp | W | TGG |
| Tyrosine | Tyr | Y | TAC TAT |
| Valine | Val | V | GTA, GTC, GTG, GTT |

A “parent” polypeptide amino acid sequence is the starting sequence for introduction of mutations (e.g. by introducing one or more amino acid substitutions, insertions, deletions, or a combination thereof) to the sequence, resulting in “variants” of the parent polypeptide amino acid sequences. A parent incudes: A wild-type polypeptide amino acid sequence or synthetically generated polypeptide amino acid sequence that is used as starting sequence for introduction of (further) changes.

A “variant polypeptide” refers to an enzyme that differs from its parent in its amino acid sequence. While the definition below describes variants in the context of amino acid changes, nucleic acids may be similarly modified, e.g. by substitutions. The parent polypeptide sequence of this invention is SEQ ID NO: 1 (encoded by SEQ ID NO:13), SEQ ID NO:2 (encoded by SEQ ID NO: 14), SEQ ID NO:3 (encoded by SEQ ID NO: 15), SEQ ID NO:4 (encoded by SEQ ID NO: 16), SEQ ID NO:5 (encoded by SEQ ID NO: 17), SEQ ID NO:6 (encoded by SEQ ID NO: 18), SEQ ID NO:7 (encoded by SEQ ID NO: 19), SEQ ID NO:8 (encoded by SEQ ID NO:20), SEQ ID NO:9 (encoded by SEQ ID NO:21), or SEQ ID NO: 10 (encoded by SEQ ID NO:22).

A “mature polypeptide” means an enzyme in its final form including any post-transitional modifications, glycosylation, phosphorylation, truncation, N-terminal modifications, C-terminal modifications, signal sequence deletion. A mature polypeptide can vary depending upon the expression system, vector, promoter, and/or production process.

A “synthetic” or “artificial” compound is produced by in vitro chemical or enzymatic synthesis. The term “non-naturally occurring” refers to a (poly)nucleotide, amino acid, (poly)peptide, enzyme, protein, cell, organism, or other material that is not present in its original naturally occurring environment or source.

Variant polynucleotide and variant polypeptide sequences may be defined by their sequence identity when compared to a parent sequence. Sequence identity usually is provided as “% sequence identity” or “% identity”. For calculation of sequence identities, in a first step a sequence alignment has to be produced. According to this invention, a pairwise global alignment has to be produced, meaning that two sequences have to be aligned over their complete length, which is usually produced by using a mathematical approach, called alignment algorithm.

According to the invention, the alignment is generated by using the algorithm of Needleman and Wunsch (J. Mol. Biol. (1979) 48, p. 443-453). Preferably, the program “NEEDLE” (The European Molecular Biology Open Software Suite (EMBOSS)) is used for the purposes of the current invention, with using the programs default parameter (polynucleotides: gap open=10.0, gap extend=0.5 and matrix=EDNAFULL; polypeptides: gap open=10.0, gap extend=0.5 and matrix=EBLOSUM62).

After aligning two sequences, in a second step, an identity value is determined from the alignment produced.

For this purpose, the %-identity is calculated by dividing the number of identical residues by the length of the alignment region which is showing the two aligned sequences over their complete length multiplied with 100: %-identity=(identical residues/length of the alignment region which is showing the two aligned sequences over their complete length)*100.

For calculating the percent identity of two nucleic acid sequences the same applies as for the calculation of percent identity of two amino acid sequences with some specifications. For nucleic acid sequences encoding for a protein the pairwise alignment shall be made over the complete length of the coding region of the sequence of this invention from start to stop codon excluding introns. Introns present in the other sequence, to which the sequence of this invention is compared, may also be removed for the pairwise alignment. Percent identity is then calculated by %-identity=(identical residues/length of the alignment region which is showing the two aligned sequences from start to stop codon excluding introns over their complete length)*100.

Moreover, the preferred alignment program for nucleic acid sequences implementing the Needleman and Wunsch algorithm (J. Mol. Biol. (1979) 48, p. 443-453) is “NEEDLE” (The European Molecular Biology Open Software Suite (EMBOSS)) with the programs default parameters (gapopen=10.0, gapextend=0.5 and matrix=EDNAFULL).

Sequences, having identical or similar regions with a sequence of this invention, and which shall be compared with a sequence of this invention to determine % identity, can easily be identified by various ways that are within the skill in the art, for instance, using publicly available computer methods and programs such as BLAST, BLAST-2, available for example at NCBI.

Variant polypeptides may be defined by their sequence similarity when compared to a parent sequence. Sequence similarity usually is provided as “% sequence similarity” or “%-similarity”. % sequence similarity takes into account that defined sets of amino acids share similar properties, e.g by their size, by their hydrophobicity, by their charge, or by other characteristics. Herein, the exchange of one amino acid with a similar amino acid may be called “conservative mutation”. Similar amino acids according to the invention are defined as follows, which shall also apply for determination of %-similarity according to this invention, which is also in accordance with the BLOSUM62 matrix as for example used by program “NEEDLE”, which is one of the most used amino acids similarity matrix for database searching and sequence alignments:

Amino acid A is similar to amino acids S

Amino acid D is similar to amino acids E; N

Amino acid E is similar to amino acids D; K; Q

Amino acid F is similar to amino acids W; Y

Amino acid H is similar to amino acids N; Y

Amino acid I is similar to amino acids L; M; V

Amino acid K is similar to amino acids E; Q; R

Amino acid L is similar to amino acids I; M; V

Amino acid M is similar to amino acids I; L; V

Amino acid N is similar to amino acids D; H; S

Amino acid Q is similar to amino acids E; K; R

Amino acid R is similar to amino acids K; Q

Amino acid S is similar to amino acids A; N; T

Amino acid T is similar to amino acids S

Amino acid V is similar to amino acids I; L; M

Amino acid W is similar to amino acids F; Y

Amino acid Y is similar to amino acids F; H; W

Conservative amino acid substitutions may occur over the full length of the sequence of a polypeptide sequence of a functional protein such as an enzyme. In one embodiment, such mutations are not pertaining the functional domains of an enzyme. In one embodiment, conservative mutations are not pertaining the catalytic centers of an enzyme.

For calculation of sequence similarity, in a first step a sequence alignment has to be produced as described above. After aligning two sequences, in a second step, a similarity value is determined from the alignment produced.

For this purpose, the %-similarity is calculated by dividing the number of identical residues plus the number of similar residues by the length of the alignment region which is showing the two aligned sequences over their complete length multiplied with 100: %-similarity=[(identical residues+similar residues)/length of the alignment region which is showing the two aligned sequences over their complete length]*100.

The invention relates to a polypeptide having amylase activity comprising an amino acid sequence that is at least 80% identical, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the full length amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, or SEQ ID NO:10.

The invention relates to a polypeptide having amylase activity comprising an amino acid sequence that is at least 80% identical, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the full length amino acid sequence of SEQ ID NO:2, SEQ ID NO:3, SEQ ID NO:4, SEQ ID NO:5, SEQ ID NO:6, SEQ ID NO:7, SEQ ID NO:8, SEQ ID NO:9, or SEQ ID NO:10.

In another embodiment, the invention relates to a polypeptide having amylase activity comprising an amino acid sequence that is at least 90%, at least 91%, at least 92%, at least 93%, at least 94%, at least 95%, at least 96%, at least 97%, at least 98%, at least 99%, or 100% identical to the full length amino acid sequence of SEQ ID NO:6.

In another embodiment, the invention relates to a polypeptide having amylase activity comprising an amino acid sequence that is at least 90%, at least 91%, at least 92%, at least 93%, at least 94%, at least 95%, at least 96%, at least 97%, at least 98%, at least 99%, or 100% identical to the full length amino acid sequence of SEQ ID NO:10.

The invention further relates to a polynucleotide encoding a variant polypeptide of the invention. The terms “polynucleotide(s)”, “nucleic acid sequence(s)”, “nucleotide sequence(s)”, “nucleic acid(s)”, “nucleic acid molecule” are used interchangeably herein and refer to nucleotides, either ribonucleotides or deoxyribonucleotides or a combination of both, in a polymeric unbranched form of any length. A “gene” is a DNA segment carrying a certain genetic information.

A “parent” polynucleotide acid sequence is the starting sequence for introduction of mutations to the sequence, resulting in “variants” of said parent polynucleotide sequence. A “variant polynucleotide” refers to a polynucleotide that encodes an enzyme and the variant polynucleotide differs from its parent polynucleotide in its nucleic acid sequence.

The polynucleotide of the invention in one aspect has a nucleic acid sequence which is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical when compared to the full length polynucleotide sequence of SEQ ID NO:13, SEQ ID NO: 14, SEQ ID NO: 15 SEQ ID NO: SEQ ID NO:16, SEQ ID NO:17, SEQ ID NO:18, SEQ ID NO:19, SEQ ID NO:20, SEQ ID NO:21, or SEQ ID NO:22.

The polynucleotide of the invention in one aspect has a nucleic acid sequence which is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical when compared to the full length polynucleotide sequence of SEQ ID NO:13.

The polynucleotide of the invention in one aspect has a nucleic acid sequence which is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical when compared to the full length polynucleotide sequence of SEQ ID NO:22.

In one embodiment, the invention relates to a polypeptide having amylase activity which is encoded by a polynucleotide having a nucleic acid sequence at least 80% identical, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the full length polynucleotide sequence of SEQ ID NO:13, SEQ ID NO: 14, SEQ ID NO:15, SEQ ID NO:16, SEQ ID NO:17, SEQ ID NO:18, SEQ ID NO:19, SEQ ID NO:20, SEQ ID NO:21, or SEQ ID NO:22.

In one embodiment, the invention relates to a polypeptide having amylase activity which is encoded by a polynucleotide having a nucleic acid sequence at least 90%, at least 91%, at least 92%, at least 93%, at least 94%, at least 95%, at least 96%, at least 97%, at least 98%, at least 99%, or 100% identical to the full length polynucleotide sequence of SEQ ID NO:18.

Preferably, the polynucleotide is a codon-optimized polynucleotide for improving expression in a specific host cell.

In one embodiment, the invention relates to a polynucleotide encoding a variant polypeptide having amylase activity which has at least at least 80% identical, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO:2, SEQ ID 40 NO:3, SEQ ID NO:4, SEQ ID NO:5, SEQ ID NO:6, SEQ ID NO:7, SEQ ID NO:8, SEQ ID NO:9, or SEQ ID NO:10.

In one embodiment, the invention relates to a polynucleotide encoding a variant polypeptide having amylase activity which has at least at least 80% identical, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 1. In one embodiment, the variant polypeptide comprises one or more amino acid residue insertion, deletion, substitution, or combinations thereof to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, or SEQ ID NO:10.

In one embodiment, the variant polypeptide comprises one or more amino acid residue insertion, deletion, substitution, or combinations thereof to the amino acid sequence of SEQ ID NO:2, SEQ ID NO:3, SEQ ID NO:4, SEQ ID NO:5, SEQ ID NO:6, SEQ ID NO:7, SEQ ID NO:8, SEQ ID NO:9, or SEQ ID NO:10.

In one embodiment, the variant polypeptide comprises one or more amino acid residue substitution to the amino acid sequence of SEQ ID NO:2, SEQ ID NO:3, SEQ ID NO:4, SEQ ID NO:5, SEQ ID NO:6, SEQ ID NO:7, SEQ ID NO:8, SEQ ID NO:9, or SEQ ID NO:10.

In one embodiment, the variant polypeptide may comprise one or more amino acid residue substitution to the amino acid sequence of SEQ ID NO:6.

In one embodiment, the variant polypeptide may comprise one or more amino acid residue substitution to the amino acid sequence of SEQ ID NO:10.

“Substitutions” are described by providing the original amino acid followed by the number of the position within the amino acid sequence, followed by the substituted amino acid. A specific amino acid residue may be substituted with any of the 19 amino acid residues different from the original one. For example, the substitution of histidine at position 120 with alanine is designated as “His120Ala” or “H120A”.

Amino acid deletions are described by providing the original amino acid of the parent enzyme followed by the number of the position within the amino acid sequence, followed by *. Accordingly, the deletion of glycine at position 150 is designated as “Gly150* or G150*”. Alternatively, deletions are indicated by e.g. “deletion of D183 and G184”.

Amino acid insertions are described by providing the original amino acid of the parent enzyme followed by the number of the position within the amino acid sequence, followed by the original amino acid and the additional amino acid. For example, an insertion at position 180 of lysine next to glycine is designated as “Gly180GyLys” or “G180GK”. When more than one amino acid residue is inserted, such as e.g. a Lys and Ala after Gly180 this may be indicated as: Gly180GlyLysAla or G195GKA.

In cases where a substitution and an insertion occur at the same position, this may be indicated as S99SD+S99A or in short S99AD.

Enzyme variants comprising an amino acid sequence and multiple (combinations) of amino acid substitutions may be separated by space, (R170Y G195E); a comma (R170Y, G195E); a “−”, a “+”, or a combination of “−” and “+” (A36N-A71N+A136E+A148D+G209S+E253V). For example: K24E, D25P, L27H, A141R, G2031, S220L, S398P; represent a combination of seven different amino acid residues substitutions when compared to a parent amino acid sequence.

The one or more amino acid substitution of the variant polypeptides can be one or more conservative amino acid substitution. A “conservative amino acid substitution” or “related amino acid” means replacement of one amino acid residue in an amino acid sequence with a different amino acid residue having a similar property at the same position compared to the parent amino acid sequence. Some examples of a conservative amino acid substitution include but are not limited to replacing a positively charged amino acid residue with a different positively charged amino acid residue; replacing a polar amino acid residue with a different polar amino acid residue; replacing a non-polar amino acid residue with a different non-polar amino acid residue, replacing a basic amino acid residue with a different basic amino acid residue, or replacing an aromatic amino acid residue with a different aromatic amino acid residue.

In another embodiment, the variant polypeptide comprises one or more amino acid residue insertion, deletion, or substitution or combinations thereof, wherein the one or more amino acid residue insertion, deletion, or substitution or combinations thereof is at or after the amino acid residue position number selected from the group consisting of: 36, 39, 67, 71, 88, 93, 101, 103, 105, 106, 108, 119, 120, 121, 122, 123, 124, 127, 130, 135, 136, 138, 148, 178, 195, 199, 202, 209, 213, 216, 221, 233, 241, 248, 253, 285, 287, 305, 309, 322, 331, 340, 342, 344, 346, 354, 355, 361, 363, 364, 365, 371, 376, 378, 380, 381, 384, 385, 404, 410, 412, and 416 of the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, or SEQ ID NO:10 and wherein the variant polypeptide comprises amylase activity.

In another embodiment, the variant polypeptide comprises one or more amino acid residue insertion, deletion, substitution, or combinations thereof, wherein the one or more amino acid residue substitution is at or after the amino acid residue position number selected from the group consisting of: 36, 39, 67, 71, 88, 93, 101, 103, 105, 106, 108, 119, 120, 121, 122, 123, 124, 127, 130, 135, 136, 138, 148, 178, 195, 199, 202, 209, 213, 216, 221, 233, 241, 248, 253, 285, 287, 305, 309, 322, 331, 340, 342, 344, 346, 354, 355, 361, 363, 364, 365, 371, 376, 378, 380, 381, 384, 385, 404, 410, 412, and 416 of the amino acid sequence of SEQ ID NO:6.

In another embodiment, the present invention is directed to a polypeptide having amylase activity comprising an amino acid sequence that is at least 80% identical, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the full length amino acid sequence of SEQ ID NO:1, and wherein the polypeptide has a C-terminal deletion. Preferably, the C-terminal deletion is a deletion of at least 100, at least 150, at least 200, at least 250, at least 300, at least 350, at least 400, at least 401, at least 404, at least 407, at least 408, at least 409, at least 410, at least 411, at least 450, at least 500, at least 550, or at least 600 C-terminal amino acid residues. Preferably, the C-terminal deletion is a deletion of between 1-500, 50-400, 100-400, 150-400, 200-400, 250-400, 300-400, 350-400, 300-410, 350-410, 370-410, or 390-410 C-terminal amino acids.

In another embodiment, the present invention is directed to a polypeptide having amylase activity comprising an amino acid sequence that is at least 80% identical, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the full length amino acid sequence of SEQ ID NO:1, and wherein the polypeptide has a C-terminal deletion as described herein, and/or wherein the polypeptide comprises one or more amino acid residue insertions, deletions, substitutions, or any combinations thereof to the amino acid sequence of SEQ ID NO:1, wherein the one or more amino acid residue insertions, deletions, substitutions, or any combinations thereof is at the amino acid residue position number selected from the group consisting of: 36, 39, 67, 71, 88, 93, 101, 103, 105, 106, 108, 119, 120, 121, 122, 123, 124, 127, 130, 135, 136, 138, 148, 178, 195, 199, 202, 209, 213, 216, 221, 233, 241, 248, 253, 285, 287, 305, 309, 322, 331, 340, 342, 344, 346, 354, 355, 361, 363, 364, 365, 371, 376, 378, 380, 381, 384, 385, 404, 410, 412, and 416 of the amino acid sequence of SEQ ID NO:1.

In another embodiment, the present invention is directed to a polypeptide having amylase activity comprising an amino acid sequence that is at least 80% identical, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the full length amino acid sequence of SEQ ID NO:1, and wherein the polypeptide has a C-terminal deletion as described herein, and wherein the C-terminal truncated polypeptide comprises one or more amino acid residue insertions, deletions, substitutions, or any combinations thereof to the amino acid sequence of SEQ ID NO:1, wherein the one or more amino acid residue insertions, deletions, substitutions, or any combinations thereof is at the amino acid residue position number selected from the group consisting of: 36, 39, 67, 71, 88, 93, 101, 103, 105, 106, 108, 119, 120, 121, 122, 123, 124, 127, 130, 135, 136, 138, 148, 178, 195, 199, 202, 209, 213, 216, 221, 233, 241, 248, 253, 285, 287, 305, 309, 322, 331, 340, 342, 344, 346, 354, 355, 361, 363, 364, 365, 371, 376, 378, 380, 381, 384, 385, 404, 410, 412, and 416 of the amino acid sequence of SEQ ID NO:1.

In another aspect, the present invention related to a polypeptide having amylase activity, which is encoded by a polynucleotide having a nucleic acid sequence at least 80% identical, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the full length polynucleotide sequence of SEQ ID NO:13, wherein the polypeptide comprises one or more amino acid residue insertion, deletion, or substitution or combinations thereof at or after the amino acid residue position number selected from the group consisting of: 36, 39, 67, 71, 88, 93, 101, 103, 105, 106, 108, 119, 120, 121, 122, 123, 124, 127, 130, 135, 136, 138, 148, 178, 195, 199, 202, 209, 213, 216, 221, 233, 241, 248, 253, 285, 287, 305, 309, 322, 331, 340, 342, 344, 346, 354, 355, 361, 363, 364, 365, 371, 376, 378, 380, 381, 384, 385, 404, 410, 412, and 416 of the amino acid sequence of SEQ ID NO: 1.

In another embodiment, the present invention relates to a polypeptide having amylase activity comprising an amino acid sequence that is at least 80% identical, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the full length amino acid sequence of SEQ ID NO:1, wherein the polypeptide comprises one or more amino acid residue insertion, deletion, or substitution or combinations thereof at or after the amino acid residue position number selected from the group consisting of: 36, 39, 67, 71, 88, 93, 101, 103, 105, 106, 108, 119, 120, 121, 122, 123, 124, 127, 130, 135, 136, 138, 148, 178, 195, 199, 202, 209, 213, 216, 221, 233, 241, 248, 253, 285, 287, 305, 309, 322, 331, 340, 342, 344, 346, 354, 355, 361, 363, 364, 365, 371, 376, 378, 380, 381, 384, 385, 404, 410, 412, and 416 of the amino acid sequence of SEQ ID NO: 1.

In another embodiment, the variant polypeptide comprises one or more amino acid substitution, wherein the one or more amino acid substitution is a substitution selected from the group consisting of: A36N, L39A, L39R, K67R, A71N, Q88H, S93Q, S101P, S101R, N103D, G105S, G105D, G105H, G105Y, S106F, S108D, H119E, H119S, A120P, G121C, S122E, S122D, S122L, S122N, S122P, L123V, L1231, T124N, A127S, A127T, A127L, N130W, G135C, A136E, L1381, L138N, L138M, L138P, L138Q, L138K, L138H, A148D, S178D, Y195F, 1199W, T202E, T202P, T202A, G209S, G209E, G209C, G213D, G213E, G213C, A216V, K221R, Q233R, Q233L, G241D, A248P, E253C, E253V, E2531, F285Y, L287V, T305S, 1309L, S322T, N331S, N340D, A342V, G344H, S346G, G354T, N355R, G361S, G363C, A364V, K365A, K365Q, N371Y, T376A, S378R, S380K, S380R, L381A, L381T, G384Q, G384Y, L385Q, T4041, S410N, S410T, M412L, and N416E of the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, or SEQ ID NO:10.

In one embodiment, the variant polypeptide is a polypeptide having the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, or SEQ ID NO:10, and one or more amino acid substitutions selected from the group consisting of: S378R; A127T; T202E; L39A; N355R; S380K; S410N; A342V; A127L; A127S; T305S; S122N; S122P; and L123V. In one embodiment, the variant polypeptide is a polypeptide having the amino acid sequence of SEQ ID NO:6, and one or more amino acid substitutions selected from the group consisting of: S378R; A127T; T202E; L39A; N355R; S380K; S410N; A342V; A127L; A127S; T305S; S122N; S122P; and L123V. In another embodiment, the variant polypeptide improves the thermostability. In one embodiment, the variant polypeptide that improves the thermostability is a polypeptide having the amino acid sequence of SEQ ID NO:6, and one or more amino acid substitutions selected from the group consisting of: S378R; A127T; T202E; L39A; N355R; S380K; S410N; A342V; A127L; A127S; T305S; S122N; S122P; and L123V.

In one embodiment, the variant polypeptide is a polypeptide having the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, or SEQ ID NO:10, and one or more amino acid substitutions selected from the group consisting of: S378R; A364V; L381A; L39A; T202P; L381T; A248P; S380K; T305S; G384Y; and M412L. In one embodiment, the variant polypeptide is a polypeptide having the amino acid sequence of SEQ ID NO:6, and one or more amino acid substitutions selected from the group consisting of: S378R; A364V; L381A; L39A; T202P; L381T; A248P; S380K; T305S; G384Y; and M412L. In another embodiment, the variant polypeptide improves the performance on cloth (preferably, stain removal from CFT-CS28 and EMPA161 at both 25C and 40C). In one embodiment, the variant polypeptide that improves the performance on cloth (preferably, stain removal from CFT-CS28 and EMPA161 at both 25C and 40C) is a polypeptide having the amino acid sequence of SEQ ID NO:6, and one or more amino acid substitutions selected from the group consisting of: S378R; A364V; L381A; L39A; T202P; L381T; A248P; S380K; T305S; G384Y; and M412L.

In one embodiment, the variant polypeptide is a polypeptide having the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, or SEQ ID NO:10, and one or more amino acid substitutions selected from the group consisting of: A127T; S101P; A127L; A127S; S122P; and S122L. In one embodiment, the variant polypeptide is a polypeptide having the amino acid sequence of SEQ ID NO:6, and one or more amino acid substitutions selected from the group consisting of: A127T; S101P; A127L; A127S; S122P; and S122L. In another embodiment, the variant polypeptide improves the stability of the amylase in the present of protease. In one embodiment, the variant polypeptide that improves the stability of the amylase in the present of protease is a polypeptide having the amino acid sequence of SEQ ID NO:6, and one or more amino acid substitutions selected from the group consisting of: A127T; S101P; A127L; A127S; S122P; and S122L.In one embodiment, the variant polypeptide is a polypeptide having the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, or SEQ ID NO:10, and one or more amino acid substitutions selected from the group consisting of: T202E; N355R; S410N; A342V; K365Q; S122N; and L123V.

In one embodiment, the variant polypeptide is a polypeptide having the amino acid sequence of SEQ ID NO:6, and one or more amino acid substitutions selected from the group consisting of: T202E; N355R; S410N; A342V; K365Q; S122N; and L123V.

In another embodiment, the variant polypeptide improves the performance on cloth (preferably, stain removal from CFT-CS28 and EMPA161 at both 25C and 40C) and improves the stability of the amylase in the present of protease. In one embodiment, the variant polypeptide that improves the performance on cloth (preferably, stain removal from CFT-CS28 and EMPA161 at both 25C and 40C) and improves the stability of the amylase in the present of protease is a polypeptide having the amino acid sequence of SEQ ID NO:6, and one or more amino acid substitutions selected from the group consisting of: T202E; N355R; S410N; A342V; K365Q; S122N; and L123V.

In another embodiment, the polypeptide comprises an amino acid sequence that is at least 80% identical to the full length amino acid sequence as set forth in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, or SEQ ID NO:10; and one or more amino acid substitution to the amino acid sequence of SEQ ID NO:1, wherein the one or more amino acid substitution is selected from the group consisting of: A36N, L39A, L39R, K67R, A71N, Q88H, S93Q, S101P, S101R, N103D, G105S, G105D, G105H, G105Y, S106F, S108D, H119E, H119S, A120P, G121C, S122E, S122D, S122L, S122N, S122P, L123V, L1231, T124N, A127S, A127T, A127L, N130W, G135C, A136E, L1381, L138N, L138M, L138P, L138Q, L138K, L138H, A148D, S178D, Y195F, 1199W, T202E, T202P, T202A, G209S, G209E, G209C, G213D, G213E, G213C, A216V, K221R, Q233R, Q233L,G241D, A248P, E253C, E253V, E2531, F285Y, L287V, T305S, I309L,S322T, N331S, N340D, A342V, G344H, S346G, G354T, N355R, G361S, G363C, A364V, K365A, K365Q, N371Y, T376A, S378R, S380K, S380R, L381A, L381T, G384Q, G384Y, L385Q, T4041, S410N, S410T, M412L, and N416E of the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, or SEQ ID NO:10.

In another embodiment, the polypeptide comprises an amino acid sequence that is at least at least 90%, at least 91%, at least 92%, at least 93%, at least 94%, at least 95%, at least 96%, at least 97%, at least 98%, or at least 99% identical to the full length amino acid sequence as set forth in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, or SEQ ID NO:10; and one or more amino acid substitution to the amino acid sequence of SEQ ID NO:1, wherein the one or more amino acid substitution is selected from the group consisting of: A36N, L39A, L39R, K67R, A71N, Q88H, S93Q, S101P, S101R, N103D, G105S, G105D, G105H, G105Y, S106F, S108D, H119E, H119S, A120P, G121C, S122E, S122D, S122L, S122N, S122P, L123V, L1231, T124N, A127S, A127T, A127L, N130W, G135C, A136E, L1381, L138N, L138M, L138P, L138Q, L138K, L138H, A148D, S178D, Y195F, 1199W, T202E, T202P, T202A, G209S, G209E, G209C, G213D, G213E, G213C, A216V, K221R, Q233R, Q233L, G241D, A248P, E253C, E253V, E2531, F285Y, L287V, T305S, I309L, S322T, N331S, N340D, A342V, G344H, S346G, G354T, N355R, G361S, G363C, A364V, K365A, K365Q, N371Y, T376A, S378R, S380K, S380R, L381A, L381T, G384Q, G384Y, L385Q, T4041, S410N, S410T, M412L, and N416E of the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, or SEQ ID NO:10.

In another embodiment, the polypeptide comprises an amino acid sequence that is at least at least 90%, at least 91%, at least 92%, at least 93%, at least 94%, at least 95%, at least 96%, at least 97%, at least 98%, or at least 99% identical to the full length amino acid sequence as set forth in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, or SEQ ID NO:10; and one or more amino acid substitution to the amino acid sequence of SEQ ID NO:1, wherein the one or more amino acid substitution is selected from the group consisting of: A36N, L39A, L39R, K67R, A71N, Q88H, S93Q, S101P, S101R, N103D, G105S, G105D, G105H, G105Y, S106F, S108D, H119E, H119S, A120P, G121C, S122E, S122D, S122L, S122N, S122P, L123V, L1231, T124N, A127S, A127T, A127L, N130W, G135C, A136E, L1381, L138N, L138M, L138P, L138Q, L138K, L138H, A148D, S178D, Y195F, 1199W, T202E, T202P, T202A, G209S, G209E, G209C, G213D, G213E, G213C, A216V, K221R, Q233R, Q233L, G241D, A248P, E253C, E253V, E2531, F285Y, L287V, T305S, I309L, S322T, N331S, N340D, A342V, G344H, S346G, G354T, N355R, G361S, G363C, A364V, K365A, K365Q, N371Y, T376A, S378R, S380K, S380R, L381A, L381T, G384Q, G384Y, L385Q, T4041, S410N, S410T, M412L, and N416E of the amino acid sequence of SEQ ID NO:6.

Another embodiment of the present invention is a variant polypeptide having amylase activity, wherein the variant polypeptide is an amino acid sequence that is at least 80% identical, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the full length amino acid sequence as set forth in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, or SEQ ID NO:10, wherein the variant polypeptide has a combination of amino acid modifications to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, or SEQ ID NO:10; and the combination of amino acid modifications is selected from the group consisting of a modifications as described in the Examples as: Table 2, Table 3, Table 4, Table 5, Table 6, Table 7, Table 8, and Table 9.

Another embodiment of the present invention is a variant polypeptide having amylase activity, wherein the variant polypeptide is an amino acid sequence that is at least 80% identical, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the full length amino acid sequence as set forth in of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, or SEQ ID NO:10, wherein the variant polypeptide has a combination of amino acid modifications to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, or SEQ ID NO:10, and the combination of amino acid modifications is G105S S108D, T127S. G209C, and Q233R.

Another embodiment of the present invention is a variant polypeptide having amylase activity, wherein the variant polypeptide is an amino acid sequence that is at least 80% identical, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the full length amino acid sequence as set forth in SEQ ID NO:10, wherein the variant polypeptide has a combination of amino acid modifications to the amino acid sequence of SEQ ID NO:10, and the combination of amino acid modifications is G105S, S108D, T127S, G209C, and Q233R.

Another embodiment of the present invention is a variant polypeptide having amylase activity, wherein the variant polypeptide is an amino acid sequence that is at least 80% identical, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the full length amino acid sequence as set forth in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, or SEQ ID NO:10, wherein the variant polypeptide has a combination of amino acid modifications to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, or SEQ ID NO:10, and the combination of amino acid modifications is G105S, S108D, T127S, and G209C.

Another embodiment of the present invention is a variant polypeptide having amylase activity, wherein the variant polypeptide is an amino acid sequence that is at least 80% identical, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the full length amino acid sequence as set forth in SEQ ID NO:10, wherein the variant polypeptide has a combination of amino acid modifications to the amino acid sequence of SEQ ID NO:10, and the combination of amino acid modifications is G105S, S108D, T127S, and G209C.

Another embodiment of the present invention is a variant polypeptide having amylase activity, wherein the variant polypeptide is an amino acid sequence that is at least 80% identical, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the full length amino acid sequence as set forth in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, or SEQ ID NO:10, wherein the variant polypeptide has a combination of amino acid modifications to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, or SEQ ID NO:10, and the combination of amino acid modifications is G105S S108D, T127S. G209E, Q233R, and N331S.

Another embodiment of the present invention is a variant polypeptide having amylase activity, wherein the variant polypeptide is an amino acid sequence that is at least 80% identical, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the full length amino acid sequence as set forth in SEQ ID NO:10, wherein the variant polypeptide has a combination of amino acid modifications to the amino acid sequence of SEQ ID NO:10, and the combination of amino acid modifications is G105S, S108D, T127S, G209E, Q233R, and N331S.

Another embodiment of the present invention is a variant polypeptide having amylase activity, wherein the variant polypeptide is an amino acid sequence that is at least 80% identical, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the full length amino acid sequence as set forth in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, or SEQ ID NO:10, wherein the variant polypeptide has a combination of amino acid modifications to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, or SEQ ID NO:10, and the combination of amino acid modifications is Q88H, L138M, E253V, G105S, T127S, 1309L, T376A, A36N, A71N, A148D, and G209S.

Another embodiment of the present invention is a variant polypeptide having amylase activity, wherein the variant polypeptide is an amino acid sequence that is at least 80% identical, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the full length amino acid sequence as set forth in SEQ ID NO:10, wherein the variant polypeptide has a combination of amino acid modifications to the amino acid sequence of SEQ ID NO:10, and the combination of amino acid modifications is Q88H, L138M, E253V, G105S, T127S, 1309L, T376A, A36N, A71N, A148D, and G209S.

Another embodiment of the present invention is a variant polypeptide having amylase activity, wherein the variant polypeptide is an amino acid sequence that is at least 80% identical, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the full length amino acid sequence as set forth in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, or SEQ ID NO:10, wherein the variant polypeptide has a combination of amino acid modifications to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, or SEQ ID NO:10, and the combination of amino acid modifications is Q88H, L138M, E253V, G105S, T127S, A36N, A71N, A148D, G209S, K221R, N340D, and S322T.

Another embodiment of the present invention is a variant polypeptide having amylase activity, wherein the variant polypeptide is an amino acid sequence that is at least 80% identical, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the full length amino acid sequence as set forth in SEQ ID NO:10, wherein the variant polypeptide has a combination of amino acid modifications to the amino acid sequence of SEQ ID NO:10, and the combination of amino acid modifications is Q88H, L138M, E253V, G105S, T127S, A36N, A71N, A148D, G209S, K221R, N340D, and S322T.

Another embodiment of the present invention is a variant polypeptide having amylase activity, wherein the variant polypeptide is an amino acid sequence that is at least 80% identical, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the full length amino acid sequence as set forth in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, or SEQ ID NO:10, wherein the variant polypeptide has a combination of amino acid modifications to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, or SEQ ID NO:10, and the combination of amino acid modifications is Q88H, L138M, E253V, G105S, T127S, A36N, A71N, A148D, and G209S.

Another embodiment of the present invention is a variant polypeptide having amylase activity, wherein the variant polypeptide is an amino acid sequence that is at least 80% identical, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the full length amino acid sequence as set forth in SEQ ID NO:10, wherein the variant polypeptide has a combination of amino acid modifications to the amino acid sequence of SEQ ID NO:10, and the combination of amino acid modifications is Q88H, L138M, E253V, G105S, T127S, A36N, A71N, A148D, and G209S.

Another embodiment of the present invention is a variant polypeptide having amylase activity, wherein the variant polypeptide is an amino acid sequence that is at least 80% identical, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the full length amino acid sequence as set forth in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, or SEQ ID NO:10, wherein the variant polypeptide has a combination of amino acid modifications to the amino acid sequence of SEQ ID NO: SEQ ID NO:2, SEQ ID NO:3, SEQ ID NO:4, SEQ ID NO:5, SEQ ID NO:6, SEQ ID NO:7, SEQ ID NO:8, SEQ ID NO:9, or SEQ ID NO:10, and the combination of amino acid modifications is Q88H, L138M, E253V, G105S, T127S, 1309L, T376A, A36N, A71N, A136E, A148D, and G209S.

Another embodiment of the present invention is a variant polypeptide having amylase activity, wherein the variant polypeptide is an amino acid sequence that is at least 80% identical, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the full length amino acid sequence as set forth in SEQ ID NO:10, wherein the variant polypeptide has a combination of amino acid modifications to the amino acid sequence of SEQ ID NO:10, and the combination of amino acid modifications is Q88H, L138M, E253V, G105S, T127S, 1309L, T376A, A36N, A71N, A136E, A148D, and G209S.

Another embodiment of the present invention is a variant polypeptide having amylase activity, wherein the variant polypeptide is an amino acid sequence that is at least 80% identical, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the full length amino acid sequence as set forth in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, or SEQ ID NO:10, wherein the variant polypeptide has a combination of amino acid modifications to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, or SEQ ID NO:10, and the combination of amino acid modifications is Q88H, L138M, E253V, G105S, T127S, A36N, A71N, A136E, A148D, and G209S.

Another embodiment of the present invention is a variant polypeptide having amylase activity, wherein the variant polypeptide is an amino acid sequence that is at least 80% identical, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the full length amino acid sequence as set forth in SEQ ID NO:10, wherein the variant polypeptide has a combination of amino acid modifications to the amino acid sequence of SEQ ID NO:10, and the combination of amino acid modifications is Q88H, L138M, E253V, G105S, T127S, A36N, A71N, A136E, A148D, and G209S.

Another embodiment of the present invention is a variant polypeptide having amylase activity, wherein the variant polypeptide is an amino acid sequence that is at least 80% identical, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the full length amino acid sequence as set forth in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, or SEQ ID NO:10, wherein the variant polypeptide has a combination of amino acid modifications to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, or SEQ ID NO:10, and the combination of amino acid modifications is Q88H, L138M, E253V, G105S, T127S, and A36N.

Another embodiment of the present invention is a variant polypeptide having amylase activity, wherein the variant polypeptide is an amino acid sequence that is at least 80% identical, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the full length amino acid sequence as set forth in SEQ ID NO:10, wherein the variant polypeptide has a combination of amino acid modifications to the amino acid sequence of SEQ ID NO:10, and the combination of amino acid modifications is Q88H, L138M, E253V, G105S, T127S, and A36N.

Another embodiment of the present invention is a variant polypeptide having amylase activity, wherein the variant polypeptide is an amino acid sequence that is at least 80% identical, to the full length amino acid sequence as set forth in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, or SEQ ID NO:10, and the variant polypeptide has an increase in enzyme activity, thermostability, pH stability, protease stability, or any combination thereof when compared to the amylase of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, or SEQ ID NO:10.

A further embodiment of the present invention is a polypeptide having amylase activity, wherein the polypeptide is an amino acid sequence that is at least 80% identical, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the full length amino acid sequence as set forth in SEQ ID NO:2, SEQ ID NO:3, SEQ ID NO:4, SEQ ID NO:5, SEQ ID NO:6, SEQ ID NO:7, SEQ ID NO:8, SEQ ID NO:9, or SEQ ID NO:10, and the polypeptide has an increase in enzyme activity, thermostability, pH-stability, protease stability, or any combination thereof when compared to the amylase of SEQ ID NO:1.

A further embodiment of the present invention is a polypeptide having amylase activity, wherein the polypeptide comprises an amino acid sequence that is at least 80% identical, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the full length amino acid sequence as set forth in SEQ ID NO:1, wherein the polypeptide comprises one or more amino acid residue insertion, deletion, or substitution or combinations thereof at or after the amino acid residue position number selected from the group consisting of: 36, 39, 67, 71, 88, 93, 101, 103, 105, 106, 108, 119, 120, 121, 122, 123, 124, 127, 130, 135, 136, 138, 148, 178, 195, 199, 202, 209, 213, 216, 221, 233, 241, 248, 253, 285, 287, 305, 309, 322, 331, 340, 342, 344, 346, 354, 355, 361, 363, 364, 365, 371, 376, 378, 380, 381, 384, 385, 404, 410, 412, and 416 of the amino acid sequence of SEQ ID NO: 1, and the polypeptide has an increase in enzyme activity, thermostability, pH-stability, protease stability, or any combination thereof when compared to the amylase of SEQ ID NO:1.

“Enzymatic activity” means at least one catalytic effect exerted by an enzyme. Enzymatic activity is expressed as units per milligram of enzyme (specific activity) or molecules of substrate transformed per minute per molecule of enzyme (molecular activity). Enzymatic activity can be specified by the enzymes actual function, e.g. proteases exerting proteolytic activity by catalyzing hydrolytic cleavage of peptide bonds, lipases exerting lipolytic activity by hydrolytic cleavage of ester bonds, amylases activity involves (endo)hydrolysis of glucosidic linkages in polysaccharides, etc.

Enzymatic activity may change during storage or operational use of the enzyme. The term “enzyme stability” relates to the retention of enzymatic activity as a function of time during storage or operation. The term “storage” herein means to indicate the fact of products or compositions or formulations being stored from the time of being manufactured to the point in time of being used in final application. Retention of enzymatic activity as a function of time during storage may be called “storage stability” herein.

To determine and quantify changes in catalytic activity of enzymes stored or used under certain conditions over time, the “initial enzymatic activity” is measured under defined conditions at time cero (100%) and at a certain point in time later (x %). By comparison of the values measured, a potential loss of enzymatic activity can be determined in its extent. The extent of enzymatic activity loss determines an enzymes stability or non-stability.

Parameters influencing the enzymatic activity of an enzyme and/or storage stability and/or operational stability are for example pH, temperature, and presence of oxidative substances.

A variant polypeptide may be active over a broad pH at any single point within the range from about pH 4.0 to about pH 12.0. The variant polypeptides enzyme may be active over a range of pH4.0 to pH 11.0, pH 4.0 to pH 10.0, pH 4.0 to pH 9.0, pH 4.0 to pH 8.0, pH 4.0 to pH 7.0, pH 4.0 to pH 6.0, or pH 4.0 to pH 5.0. The variant polypeptides may be active at pH 4.0, pH 4.1, pH 4.2, pH 4.3, pH 4.4, pH 4.5, pH 4.6, pH 4.7, pH 4.8, pH 4.9, pH 5.0, pH 5.1, pH 5.2, pH 5.3, pH 5.4, pH 5.5, pH 5.6, pH 5.7, pH 5.8, pH 5.9, pH 6.0, pH 6.1, pH 6.2, pH 6.3, pH 6.4, pH 6.5, pH 6.6, pH 6.7, pH 6.8, pH 6.9, pH 7.0, pH 7.1, pH 7.2, pH 7.3, pH 7.4, pH 7.5, pH 7.6, pH 7.7, pH 7.8, pH 7.9, pH 8.0, pH 8.1, pH 8.2, pH 8.3, pH 8.4, pH 8.5, pH 8.6 pH 8.7, pH 8.8 pH 8.9, pH 9.0, pH 9.1, pH 9.2, pH 9.3, pH 9.4, pH 9.5, pH 9.6, pH 9.7, pH 9.8, pH 9.9, pH 10.0, pH 10.1, pH 10.2, pH 10.3, pH 10.4, pH 10.5, pH 10.6, pH 10.7, pH 10.8, pH 10.9, pH 11.0, pH 11.1, pH 11.2, pH 11.3, pH 11.4, pH 11.5, pH 11.6, pH 11.7, pH 11.8, pH 11.9, pH 12.0, pH 12.1, pH 12.2, pH 12.3, pH 12.4, and pH 12.5. pH 12.6, pH 12.7, pH 12.8, pH 12.9, and higher.

A “pH stability”, refers to the ability of an enzyme to exert enzymatic activity at a specific pH range.

The variant polypeptides may be active over a broad temperature, wherein the temperature is any point in the range from about 10° C. to about 60° C. The variant polypeptides may be active at a temperature range from 10° C. to 55° C., 10° C. to 50° C., 10° C. to 45° C., 10° C. to 40° C., 10° C. to 35° C., 10° C. to 30° C., or 10° C. to 25° C. The variant polypeptides may be active at a temperature range from 20° C. to 55° C., 20° C. to 50° C., 20° C. to 45° C., 20° C. to 40° C., 20° C. to 35° C., 20° C. to 30° C., or 20° C. to 25° C. The variant polypeptides are active at a temperature of at least 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., or higher temperatures.

The terms “thermal stability” and “thermostability” refer to the ability of a protein to exert catalytic activity at a specific temperature range. Enzymes generally have a finite range of temperatures at which they exert catalytic activity. In addition to enzymes that exert catalytic activity in midrange temperatures (e.g., room temperature), there are enzymes that are capable of exerting catalytic activity in very high or very low temperatures. Thermostability may be characterized by what is known as the T₅₀ value (also called half-life, see above). The T₅₀ indicates the temperature at which 50% residual enzymatic activity is still present after thermal inactivation for a certain time when compared with a reference sample which has not undergone thermal treatment.

The terms “thermal tolerance” and “thermotolerance” refer to the ability of a protein to exert catalytic activity after exposure to a particular temperature, such as a very high or very low temperature. A thermotolerant protein may not exert catalytic activity at the exposure temperature, but will exert catalytic activity once returned to a favorable temperature.

In one embodiment, the variant polypeptides improve the thermostability. In another embodiment the variant polypeptides improve the thermostability by 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., or more degrees C. when compared to the parent polypeptide. In another embodiment, the variant polypeptide that improves the thermostability is described in Table 1, Table 2, Table 3, Table 4, Table 5, Table 6, Table 7, Table 8, and Table 9.

In one embodiment, the variant polypeptides improve the amylase residual activity in the presence of protease when compared to the parent polypeptide. In another embodiment, the variant polypeptides that improve the amylase residual activity in the presence of protease are described in Table 3, Table 4, and Table 5.

In one embodiment, the variant polypeptides improve the residual activity when compared to the parent polypeptide. In another embodiment the variant polypeptides improve the residual activity when compared to the parent polypeptide are described in Table 6, Table 7, Table 8, and Table 9.

In one embodiment, the variant polypeptide is a fragment of the full length amino acid sequence and the fragment has amylase activity.

A “Fragment”, or “subsequence” as used herein are a portion of a polynucleotide or an amino acid sequence, wherein the fragments or subsequences retain or encode for at least one functional activity of the sequence to which it is related.

The term “functional fragment” refers to any nucleic acid or amino acid sequence which comprises merely a part of the full length amino acid sequence, respectively, but still has the same or similar activity and/or function. The fragment comprises at least 70%, at least 80%, at least 85%, at least 90%, at least 95%, at least 96%, at least 97%, at least 98%, or at least 99% of the original sequence. The functional fragment comprises contiguous nucleic acids or amino acids compared to the original nucleic acid or original amino acid sequence, respectively.

In one embodiment, the variant polypeptide having amylase activity is a fragment of a polypeptide comprising an amino acid sequence that is at least 80% identical, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the full length amino acid sequence of SEQ ID NO: 1. In one embodiment, the fragment of the polypeptide comprises an amino acid sequence that is at least 80% identical, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the full length amino acid sequence of SEQ ID NO:2, SEQ ID NO:3, SEQ ID NO:4, SEQ ID NO:5, SEQ ID NO:6, SEQ ID NO:7, SEQ ID NO:8, SEQ ID NO:9, or SEQ ID NO:10, wherein the fragment has amylase activity.

The polypeptide variants can be a hybrid of more than one amylase enzymes. In one embodiment, the polypeptide variant can be a hybrid of the polypeptide variant and at least one additional enzyme selected from the group consisting of: a second amylase, a lipase, a protease, a cellulase, a laccase, a pectinase, and a nuclease, wherein the hybrid has amylase activity and enzymatic activity of the second enzyme.

In one embodiment, the present invention refers to a polypeptide comprising a hybrid of at least one polypeptide as described herein and a second polypeptide having an activity selected from the group consisting of amylase activity, protease activity, lipase activity, cellulase activity, laccase activity, pectinase activity, and nuclease activity, wherein the hybrid also has amylase activity.

In on embodiment the variant polypeptide comprises a hybrid of at least one variant polypeptide as described herein, and a second polypeptide having amylase activity, wherein the hybrid has amylase activity.

A “hybrid” or “chimeric” or “fusion protein” means that a fragment of the amino acid sequence of a first enzyme is combined with a fragment of the amino acid sequence of a second enzyme to form a hybrid enzyme wherein the hybrid has an enzyme activity.

The hybrid enzymes can be engineered with fragments from amino acid sequences of more than two enzymes. The domain of an amylase of this invention may be combined with a domain of a commercially available amylase such as: Amplify®, Duramyl™, Termamyl™, Fungamyl™, Stainzyme™, Stainzyme Plus™, Natalase™, Liquozyme X and BAN™ (from Novozymes A/S), and Rapidase™, Purastar™, Powerase™, Effectenz™ (M100 from DuPont), Preferenz™ (S1000, S110 and F1000; from DuPont), PrimaGreen™ (ALL; DuPont), Optisize™ (DuPont), to form a hybrid enzyme and the hybrid has amylase activity.

In another aspect the present invention refers to a composition comprising the polypeptide described herein. A composition may comprise combinations of the polypeptides with another enzyme. The combination of enzymes can be of the same class, for example a composition comprising a first amylase and a second amylase. Combinations of enzymes can be from a different class of enzymes, for example, a composition comprising a lipase and an amylase. Combinations of enzymes can be compositions comprising at least one amylase of the invention and one or more second enzymes. In one embodiment, the composition comprises one second enzyme, two second enzymes, three second enzymes, four second enzymes, or more than four second enzymes. In one particular embodiment, the second enzyme is selected from the group consisting of: a second amylase, a lipase, a protease, a cellulase, a laccase, a pectinase, and a nuclease, or any combination thereof.

Additional enzymes suitable for the hybrid or the composition of the present invention are further described below. In one embodiment, suitable enzymes include enzyme variants having enzymatic activity which are at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99% identical when compared to the full length polypeptide sequence of the parent enzyme as disclosed below.

Amylase

Alpha-amylase (E.C. 3.2.1.1) enzymes may perform endohydrolysis of (1->4)-alpha-D-glucosidic linkages in polysaccharides containing three or more (1->4)-alpha-linked D-glucose units. Amylase enzymes act on starch, glycogen and related polysaccharides and oligosaccharides in a random manner; reducing groups are liberated in the alpha-configuration. Other examples of amylase enzymes include: Beta-amylase (E.C. 3.2.1.2), Glucan 1,4-alpha-maltotetraohydrolase (E.C. 3.2.1.60), Isoamylase (E.C. 3.2.1.68), Glucan 1,4-alpha-maltohexaosidase (E.C. 3.2.1.98), and Glucan 1,4-alpha-maltohydrolase (E.C. 3.2.1.133).

Many amylase enzymes have been described in patents and published patent applications including, but not limited to: WO 2002/068589, WO 2002/068597, WO 2003/083054, WO 2004/091544, and WO 2008/080093.

Amylases are known to derived from Bacillus licheniformis having SEQ ID NO:2 as described in WO 95/10603. Suitable variants are those which are at least 90% identical to SEQ ID NO: 2 as described in WO 95/10603 and/or comprising one or more substitutions in the following positions:15, 23, 105, 106, 124, 128, 133, 154, 156, 178, 179, 181, 188, 190, 197, 201, 202, 207, 208, 209, 211, 243, 264, 304, 305, 391, 408, and 444 which have amylolytic activity. Such variants are described in WO 94/02597, WO 94/018314, WO 97/043424 and SEQ ID NO:4 of WO 99/019467.

Amylases are known to derived from B. stearothermophilus having SEQ ID NO:6 as described in WO 02/10355 or an amylase which is at least 90% identical thereto having amylolytic activity.

Suitable variants of SEQ ID NO:6 include those which is at least 90% identical thereto and/or further comprise a deletion in positions 181 and/or 182 and/or a substitution in position 193.

Amylases are known to derived from Bacillus sp.707 having SEQ ID NO:6 as disclosed in WO 99/19467 or an amylase which is at least 90% identical thereto having amylolytic activity.

Amylases are known from Bacillus halmapalus having SEQ ID NO:2 or SEQ ID NO:7 as described in WO 96/23872, also described as SP-722, or an amylase which is at least 90% identical to one of the sequences which has amylolytic activity.

Amylases are known to derived from Bacillus sp. DSM 12649 having SEQ ID NO:4 as disclosed in WO 00/22103 or an amylase which is at least 90% identical thereto having amylolytic activity.

Amylases are known from Bacillus strain TS-23 having SEQ ID NO:2 as disclosed in WO 2009/061380 or an amylase which is at least 90% identical thereto having amylolytic activity.

Amylases are known from Cytophaga sp. having SEQ ID NO:1 as disclosed in WO 2013/184577 or an amylase which is at least 90% identical thereto having amylolytic activity.

Amylases are known from Bacillus megaterium DSM 90 having SEQ ID NO:1 as disclosed in WO 2010/104675 or an amylase which is at least 90% identical thereto having amylolytic activity.

Amylases are known having amino acids 1 to 485 of SEQ ID NO:2 as described in WO 00/60060 or amylases comprising an amino acid sequence which is at least 96% identical with amino acids 1 to 485 of SEQ ID NO:2 which have amylolytic activity.

Amylases are also known having SEQ ID NO: 12 as described in WO 2006/002643 or amylases having at least 80% identity thereto and have amylolytic activity. Suitable amylases include those having at least 80% identity compared to SEQ ID NO:12 and/or comprising the substitutions at positions Y295F and M202LITV and have amylolytic activity.

Amylases are also known having SEQ ID NO:6 as described in WO 2011/098531 or amylases having at least 80% identity thereto having amylolytic activity. Suitable amylases include those having at least 80% identity compared to SEQ ID NO:6 and/or comprising a substitution at one or more positions selected from the group consisting of 193 [G,A,S,T or M], 195 [F,W,Y,L,I or V], 197 [F,W,Y,L,I or V], 198 [Q or N], 200 [F,W,Y,L,I or V], 203 [F,W,Y,L,I or V], 206 [F,W,Y,N,L,I,V,H,Q,D or E], 210 [F,W,Y,L,I or V], 212 [F,W,Y,L,I or V], 213 [G,A,S,T or M] and 243 [F,W,Y,L,I or V] and have amylolytic activity.

Amylases are known having SEQ ID NO:1 as described in WO 2013/001078 or amylases having at least 85% identity thereto having amylolytic activity. Suitable amylases include those having at least 85% identity compared to SEQ ID NO:1 and/or comprising an alteration at two or more (several) positions corresponding to positions G304, W140, W189, D134, E260, F262, W284, W347, W439, W469, G476, and G477 and having amylolytic activity.

Amylases are known having SEQ ID NO:2 as described in WO 2013/001087 or amylases having at least 85% identity thereto and having amylolytic activity. Suitable amylases include those having at least 85% identity compared to SEQ ID NO:2 and/or comprising a deletion of positions 181+182, or 182+183, or 183+184, which have amylolytic activity. Suitable amylases include those having at least 85% identity compared to SEQ ID NO:2 and/or comprising a deletion of positions 181+182, or 182+183, or 183+184, which comprise one or two or more modifications in any of positions corresponding to W140, W159, W167, Q169, W189, E194, N260, F262, W284, F289, G304, G305, R320, W347, W439, W469, G476 and G477 and have amylolytic activity.

Amylases also include hybrid α-amylase from above mentioned amylases as for example as described in WO 2006/066594.

Commercially available amylase enzymes include: Amplify®, Duramyl™, Termamyl™, Fungamyl™, Stainzyme™, Stainzyme Plus™, Natalase™, Liquozyme X and BAN™ (from Novozymes A/S), and Rapidase™, Purastar™, Powerase™, Effectenz™ (M100 from DuPont), Preferenz™ (S1000, S110 and F1000; from DuPont), PrimaGreen™ (ALL; DuPont), Optisize™ (DuPont).

Lipase

“Lipases”, “lipolytic enzyme”, “lipid esterase”, all refer to an enzyme of EC class 3.1.1 (“carboxylic ester hydrolase”). Lipases (E.C. 3.1.1.3, Triacylglycerol lipase) may hydrolyze triglycerides to more hydrophilic mono- and diglycerides, free fatty acids, and glycerol. Lipase enzymes usually includes also enzymes which are active on substrates different from triglycerides or cleave specific fatty acids, such as Phospholipase A (E.C. 3.1.1.4), Galactolipase (E.C. 3.1.1.26), cutinase (EC 3.1.1.74), and enzymes having sterol esterase activity (EC 3.1.1.13) and/or waxester hydrolase activity (EC 3.1.1.50).

Many lipase enzymes have been described in patents and published patent applications including, but not limited to: WO2000032758, WO2003/089620, WO2005/032496, WO2005/086900, WO200600976, WO2006/031699, WO2008/036863, WO2011/046812, and WO2014059360.

Lipases are used in detergent and cleaning products to remove grease, fat, oil, and dairy stains. Commercially available lipases include but are not limited to: Lipolase™, Lipex™, Lipolex™ and Lipoclean™ (Novozymes A/S), Lumafast (originally from Genencor) and Lipomax (GistBrocades/now DSM).

The methods for determining lipolytic activity are well-known in the literature (see e.g. Gupta et al. (2003), Biotechnol. Appl. Biochem. 37, p. 63-71). E.g. the lipase activity may be measured by ester bond hydrolysis in the substrate para-nitrophenyl palmitate (pNP-Palmitate, C:16) and releases pNP which is yellow and can be detected at 405 nm.

Protease

Enzymes having proteolytic activity are called “proteases” or “peptidases”. Proteases are active proteins exerting “protease activity” or “proteolytic activity”.

Proteases are members of class EC 3.4. Proteases include aminopeptidases (EC 3.4.11), dipeptidases (EC 3.4.13), dipeptidyl-peptidases and tripeptidyl-peptidases (EC 3.4.14), peptidyldipeptidases (EC 3.4.15), serine-type carboxypeptidases (EC 3.4.16), metallocarboxypeptidases (EC 3.4.17), cysteine-type carboxypeptidases (EC 3.4.18), omega peptidases (EC 3.4.19), serine endopeptidases (EC 3.4.21), cysteine endopeptidases (EC 3.4.22), aspartic endopeptidases (EC 3.4.23), metallo-endopeptidases (EC 3.4.24), threonine endopeptidases (EC 3.4.25), endopeptidases of unknown catalytic mechanism (EC 3.4.99).

Commercially available protease enzymes include but are not limited to Lavergy™ Pro (BASF); Alcalase®, Blaze®, Duralase™, Durazym™, Relase®, Relase® Ultra, Savinase®, Savinase® Ultra, Primase®, Polarzyme®, Kannase®, Liquanase®, Liquanase® Ultra, Ovozyme®, Coronase®, Coronase® Ultra, Neutrase®, Everlase® and Esperase® (Novozymes A/S), those sold under the tradename Maxatase®, Maxacal®, Maxapem®, Purafect®, Purafect® Prime, Purafect MA®, Purafect Ox®, Purafect OxP®, Puramax®, Properase®, FN2®, FN3®, FN4®, Excellase®, Eraser®, Ultimase®, Opticlean®, Effectenz®, Preferenz® and Optimase® (Danisco/DuPont), Axapem™ (Gist-Brocases N.V.), Bacillus lentus Alkaline Protease, and KAP (Bacillus alkalophilus subtilisin) from Kao.

At least one protease may be selected from serine proteases (EC 3.4.21). Serine proteases or serine peptidases (EC 3.4.21) are characterized by having a serine in the catalytically active site, which forms a covalent adduct with the substrate during the catalytic reaction. A serine protease may be selected from the group consisting of chymotrypsin (e.g., EC 3.4.21.1), elastase (e.g., EC 3.4.21.36), elastase (e.g., EC 3.4.21.37 or EC 3.4.21.71), granzyme (e.g., EC 3.4.21.78 or EC 3.4.21.79), kallikrein (e.g., EC 3.4.21.34, EC 3.4.21.35, EC 3.4.21.118, or EC 3.4.21.119) plasmin (e.g., EC 3.4.21.7), trypsin (e.g., EC 3.4.21.4), thrombin (e.g., EC 3.4.21.5) and subtilisin (also known as subtilopeptidase, e.g., EC 3.4.21.62), the latter hereinafter also being referred to as “subtilisin”.

A sub-group of the serine proteases tentatively designated subtilases has been proposed by Siezen et al. (1991), Protein Eng. 4:719-737 and Siezen et al. (1997), Protein Science 6:501-523. They are defined by homology analysis of more than 170 amino acid sequences of serine proteases previously referred to as subtilisin-like proteases. A subtilisin was previously often defined as a serine protease produced by Gram-positive bacteria or fungi, and according to Siezen et al. now is a subgroup of the subtilases. A wide variety of subtilases have been identified, and the amino acid sequence of a number of subtilases has been determined. For a more detailed description of such subtilases and their amino acid sequences reference is made to Siezen et al. (1997), Protein Science 6:501-523.

The subtilases may be divided into 6 sub-divisions, i.e. the subtilisin family, thermitase family, the proteinase K family, the lantibiotic peptidase family, the kexin family and the pyrolysin family.

A subgroup of the subtilases are the subtilisins which are serine proteases from the family S8 as defined by the MEROPS database (http://merops.sanger.ac.uk). Peptidase family S8 contains the serine endopeptidase subtilisin and its homologues. In subfamily S8A, the active site residues frequently occur in the motifs Asp-Thr/Ser-Gly (which is similar to the sequence motif in families of aspartic endopeptidases in clan AA), His-Gy-Thr-His and Gly-Thr-Ser-Met-AlaXaa-Pro. Most members of the peptidase family S8 are active at neutral-mildly alkali pH. Many peptidases in the family are thermostable.

Prominent members of family S8, subfamily A are:

| name | MEROPS Family S8, Subfamily A |
|---|---|
| Subtilisin Carlsberg | S08.001 |
| Subtilisin lentus | S08.003 |
| Thermitase | S08.007 |
| Subtilisin BPN’ | S08.034 |
| Subtilisin DY | S08.037 |
| Alkaline peptidase | S08.038 |
| Subtilisin ALP 1 | S08.045 |
| Subtilisin sendai | S08.098 |
| Alkaline elastase YaB | S08.157 |

Parent proteases of the subtilisin type (EC 3.4.21.62) and variants may be bacterial proteases. Said bacterial protease may be a Gram-positive bacterial polypeptide such as a Bacillus, Clostridium, Enterococcus, Geobacillus, Lactobacillus, Lactococcus, Oceanobacillus, Staphylococcus, Streptococcus, or Streptomyces protease, or a Gram-negative bacterial polypeptide such as a Campylobacter, E. coli, Flavobacterium, Fusobacterium, Helicobacter, Ilyobacter, Neisseria, Pseudomonas, Salmonella, or Ureaplasma protease. A review of this family is provided, for example, in “Subtilases: Subtilisin-like Proteases” by R. Siezen, pages 75-95 in “Subtilisin enzymes”, edited by R. Bott and C. Betzel, New York, 1996.

At least one protease may be selected from the following: subtilisin from Bacillus amyloliquefaciens BPN′ (described by Vasantha et al. (1984) J. Bacteriol. Volume 159, p. 811-819 and JA Wells et al. (1983) in Nucleic Acids Research, Volume 11, p. 7911-7925); subtilisin from Bacillus licheniformis (subtilisin Carlsberg; disclosed in E L Smith et al. (1968) in J. Biol Chem, Volume 243, pp. 2184-2191, and Jacobs et al. (1985) in Nucl. Acids Res, Vol 13, p. 8913-8926); subtilisin PB92 (original sequence of the alkaline protease PB92 is described in EP 283075 A2); subtilisin 147 and/or 309 (Esperase®, Savinase®, respectively) as disclosed in WO 89/06279; subtilisin from Bacillus lentus as disclosed in WO 91/02792, such as from Bacillus lentus DSM 5483 or the variants of Bacillus lentus DSM 5483 as described in WO 95/23221; subtilisin from Bacillus alcalophilus (DSM 11233) disclosed in DE 10064983; subtilisin from Bacillus gibsonii(DSM 14391) as disclosed in WO 2003/054184; subtilisin from Bacillus sp. (DSM 14390) disclosed in WO 2003/056017; subtilisin from Bacillus sp. (DSM 14392) disclosed in WO 2003/055974; subtilisin from Bacillus gibsonii (DSM 14393) disclosed in WO 2003/054184; subtilisin having SEQ ID NO: 4 as described in WO 2005/063974; subtilisin having SEQ ID NO: 4 as described in WO 2005/103244; subtilisin having SEQ ID NO: 7 as described in WO 2005/103244; and subtilisin having SEQ ID NO: 2 as described in application DE 102005028295.4.

At least one subtilisin may be subtilisin 309 (which might be called Savinase® herein) as disclosed as sequence a) in Table I of WO 89/06279 or a variant which is at least 80% identical thereto and has proteolytic activity.

Proteases are known as comprising the variants described in: WO 92/19729, WO 95/23221, WO 96/34946, WO 98/20115, WO 98/20116, WO 99/11768, WO 01/44452, WO 02/088340, WO 03/006602, WO 2004/03186, WO 2004/041979, WO 2007/006305, WO 2011/036263, WO 2011/036264, and WO 2011/072099. Suitable examples comprise especially protease variants of subtilisin protease derived from SEQ ID NO:22 as described in EP 1921147 (with amino acid substitutions in one or more of the following positions: 3, 4, 9, 15, 24, 27, 33, 36, 57, 68, 76, 77, 87, 95, 96, 97, 98, 99, 100, 101, 102, 103, 104, 106, 118, 120, 123, 128, 129, 130, 131, 154, 160, 167, 170, 194, 195, 199, 205, 206, 217, 218, 222, 224, 232, 235, 236, 245, 248, 252 and 274 which have proteolytic activity. In addition, a subtilisin protease is not mutated at positions Asp32, His64 and Ser221.

At least one subtilisin may have SEQ ID NO:22 as described in EP 1921147, or is a variant thereof which is at least 80% identical SEQ ID NO:22 as described in EP 1921147 and has proteolytic activity. In one embodiment, a subtilisin is at least 80% identical to SEQ ID NO:22 as described in EP 1921147 and is characterized by having amino acid glutamic acid (E), or aspartic acid (D), or asparagine (N), or glutamine (Q), or alanine (A), or glycine (G), or serine (S) at position 101 (according to BPN′ numbering) and has proteolytic activity. In one embodiment, subtilisin is at least 80% identical to SEQ ID NO:22 as described in EP 1921147 and is characterized by having amino acid glutamic acid (E), or aspartic acid (D), at position 101 (according to BPN′ numbering) and has proteolytic activity. Such a subtilisin variant may comprise an amino acid substitution at position 101, such as R101E or R101D, alone or in combination with one or more substitutions at positions 3, 4, 9, 15, 24, 27, 33, 36, 57, 68, 76, 77, 87, 95, 96, 97, 98, 99, 100, 101, 102, 103, 104, 106, 118, 120, 123, 128, 129, 130, 131, 154, 160, 167, 170, 194, 195, 199, 205, 206, 217, 218, 222, 224, 232, 235, 236, 245, 248, 252 and/or 274 (according to BPN′ numbering) and has proteolytic activity. In one embodiment, said protease comprises one or more further substitutions: (a) threonine at position 3 (3T), (b) isoleucine at position 4 (41), (c) alanine, threonine or arginine at position 63 (63A, 63T, or 63R), (d) aspartic acid or glutamic acid at position 156 (156D or 156E), (e) proline at position 194 (194P), (f) methionine at position 199 (199M), (g) isoleucine at position 205 (205I), (h) aspartic acid, glutamic acid or glycine at position 217 (217D, 217E or 217G), (i) combinations of two or more amino acids according to (a) to (h).

A suitable subtilisin may be at least 80% identical to SEQ ID NO:22 as described in EP 1921147 and is characterized by comprising one amino acid (according to (a)-(h)) or combinations according to (i) together with the amino acid 101E, 101D, 101N, 101Q, 101A, 101G, or 101S (according to BPN′ numbering) and has proteolytic activity.

In one embodiment, a subtilisin is at least 80% identical to SEQ ID NO:22 as described in EP 1921147 and is characterized by comprising the mutation (according to BPN′ numbering) R101E, or S3T+V41+V205I, or S3T+V4+R101E+V205I or S3T+V4+V199M+V205+L217D, and has proteolytic activity.

In another embodiment, the subtilisin comprises an amino acid sequence having at least 80% identity to SEQ ID NO:22 as described in EP 1921147 and being further characterized by comprising S3T+V41+S9R+A15T+V68A+D99S+R101S+A103S+1104V+N218D (according to the BPN′ numbering) and has proteolytic activity.

A subtilisin may have an amino acid sequence being at least 80% identical to SEQ ID NO:22 as described in EP 1921147 and being further characterized by comprising R101E, and one or more substitutions selected from the group consisting of S156D, L262E, Q137H, S3T, R45E,D,Q, P55N, T58W,Y,L, Q59D,M,N,T, G61 D,R, S87E, G97S, A98D,E,R, S106A,W, N117E, H120V,D,K,N, S125M, P129D, E136Q, S144W, S161T, S163A,G, Y171 L, A172S, N185Q, V199M, Y209W, M222Q, N238H, V244T, N261T,D and L262N,Q,D (as described in WO 2016/096711 and according to the BPN′ numbering), and has proteolytic activity.

Proteases according to the invention have proteolytic activity. The methods for determining proteolytic activity are well-known in the literature (see e.g. Gupta et al. (2002), Appl. Microbiol. Biotechnol. 60: 381-395). Proteolytic activity may be determined by using Succinyl-Ala-Ala-Pro-Phe-p-nitroanilide (Suc-AAPF-pNA, short AAPF; see e.g. DelMar et al. (1979), Analytical Biochem 99, 316-320) as substrate. pNA is cleaved from the substrate molecule by proteolytic cleavage, resulting in release of yellow color of free pNA which can be quantified by measuring OD405.

Cellulase

“Cellulases”, “cellulase enzymes” or “cellulolytic enzymes” are enzymes involved in hydrolysis of cellulose. Three major types of cellulases are known, namely endo-ss-1,4-glucanase (endo-1,4-P-D-glucan 4-glucanohydrolase, E.C. 3.2.1.4; hydrolyzing β-1,4-glucosidic bonds in cellulose), cellobiohydrolase (1,4-P-D-glucan cellobiohydrolase, EC 3.2.1.91), and ss-glucosidase (EC 3.2.1.21).

Cellulase enzymes have been described in patents and published patent applications including, but not limited to: WO1997/025417, WO1998/024799, WO2003/068910, WO2005/003319, and WO2009020459.

Commercially available cellulase enzymes include are Celluzyme™, Endolase™, Carezyme™, Cellusoft™, Renozyme™, Celluclean™ (from Novozymes A/S), Ecostone™, Biotouch™, Econase™, Ecopulp™ (from AB Enzymes Finland), Clazinase™, and Puradax HA™, Genencor detergent cellulase L, IndiAge™ Neutra (from Genencor International Inc./DuPont), Revitalen™ (2000 from DuPont), Primafast™ (DuPont) and KAC-500™ (from Kao Corporation).

Cellulases according to the invention have “cellulolytic activity” or “cellulase activity”. Assays for measurement of cellulolytic activity are known to those skilled in the art. For example, cellulolytic activity may be determined by virtue of the fact that cellulase hydrolyses carboxymethyl cellulose to reducing carbohydrates, the reducing ability of which is determined colorimetrically by means of the ferricyanide reaction, according to Hoffman, W. S., J. Biol. Chem. 120, 51 (1937).

Mannanase

Mannase (E.C. 3.2.1.78) enzymes hydrolyse internal β-1,4 bonds in mannose. Polymers. “Mannanase” may be an alkaline mannanase of Family 5 or 26. Mannanase enzymes are known to be derived from wild-type from Bacillus or Humicola, particularly B. agaradhaerens, B. licheniformis, B. halodurans, B. clausii, or H. insolens. Suitable mannanases are described in WO 99/064619.

Commercially available mannanase enzymes include: Mannaway® (Novozymes AIS).

Pectate Lyase

Pectate lyase (E.C. 4.2.2.2) enzymes eliminative cleavage of (1->4)-alpha-D-galacturonan to give oligosaccharides with 4-deoxy-alpha-D-galact-4-enuronosyl groups at their non-reducing ends.

Pectate lyase enzymes have been described in patents and published patent applications including, but not limited to: WO2004/090099. Pectate lyase are known to be derived from Bacillus, particularly B. licheniformis or B. agaradhaerens, or a variant derived of any of these, e.g. as described in U.S. Pat. No. 6,124,127, WO 99/027083, WO 99/027084, WO 2002/006442, WO 2002/092741, WO 2003/095638.

Commercially available pectate lyase enzymes include: Xpect™, Pectawash™ and Pectaway™ (Novozymes A/S); PrimaGreen™, EcoScour (DuPont).

Nuclease

Nuclease (EC 3.1.21.1) also known as Deoxyribonuclease 1, or DNase preforms endonucleolytic cleavage to 5′-phosphodinucleotide and 5′-phosphooligonucleotide end-products.

Nuclease enzymes have been described in patents and published patent applications including, but not limited to: U.S. Pat. No. 3,451,935, GB1300596, DE10304331, WO2015155350, WO2015155351, WO2015166075, WO2015181287, and WO2015181286.

In one aspect of the invention, at least one amylase variant of the invention is provided in combination with at least one protease. In one embodiment, a amylase variant of the invention is protease stabile in the presence of at least one protease. In one embodiment, a amylase variant of the invention has increased protease stability when compared to the respective amylase parent. In one embodiment, at least one protease is selected from subtilisin 309 as disclosed as sequence a) in Table I of WO 89/06279 or a variant thereof which is at least 80% identical thereto and has proteolytic activity. In one embodiment, a amylase variant of the invention has increased protease stability in the presence of said subtilisin 309 or a variant thereof which is at least 80% identical thereto when compared to the amylase according to SEQ ID NO: 1.

The protease may itself be stabilized by a protease stabilizer or the protease may be non-stabilized. In one embodiment, a amylase variant of the invention has increased protease stability in the presence of a non-stabilized subtilisin 309 or a non-stabilized variant thereof which is at least 80% identical thereto, when compared to the amylase according to SEQ ID NO: 1.

Method of Making

In another embodiment, the present invention refers to a method of making the variant polypeptide as described herein, comprising: providing a nucleic acid sequence encoding the polypeptide described herein, transforming the nucleic acid sequence into a host cell, cultivating the host cell to produce the variant polypeptide, and optionally purifying the variant polypeptide from the host cell.

A polynucleotide encoding a polypeptide may be “expressed”. The term “expression” or “gene expression” means the transcription of a specific gene or specific genes or specific nucleic acid construct. The term “expression” or “gene expression” in particular means the transcription of a gene or genes or genetic construct into structural RNA (e.g., rRNA, tRNA) or mRNA with or without subsequent translation of the latter into a protein. The process includes transcription of DNA and processing of the resulting mRNA product.

Industrial production of enzymes usually is done by using expression systems. “Expression system” may mean a host microorganism, expression hosts, host cell, production organism, or production strain and each of these terms can be used interchangeably. In one embodiment, the expression host is selected from the group consisting of: a bacterial expression system, a yeast expression system, a fungal expression system, and a synthetic expression system. The expression host may be a wildtype cell or a recombinant cell. “Wild-type cells” herein means cells prior to a certain modification. The term “recombinant cell” (also called “genetically modified cell” herein) refers to a cell which has been genetically altered, modified or engineered such it that exhibits an altered, modified or different genotype as compared to the wild-type cell which it was derived from. The “recombinant cell” may comprise an exogenous polynucleotide encoding a certain protein or enzyme and therefore may express said protein or enzyme.

Thus, in one embodiment, the invention is directed to a genetic construct comprising a polynucleotide encoding the amylase as described herein.

In one embodiment, the invention is directed to a host cell comprising a polynucleotide encoding the amylase as described herein.

In yet another embodiment, the present invention is directed to a method of expressing a polynucleotide, comprising the steps of
(a) providing a host cell comprising a heterologous nucleic acid construct comprising a polynucleotide encoding the amylase described herein by introducing the nucleic acid construct comprising the polynucleotide encoding the amylase as described herein into the host cell;
(b) cultivating the recombinant host cell of step (a) under conditions conductive for the expression of the polynucleotide; and
(c) optionally, recovering a protein of interest encoded by the polynucleotide.

Examples of expression systems include but are not limited to: Aspergillus niger, Aspergillus oryzae, Hansenula polymorpha, Thermomyces lanuginosus, Fusarium oxysporum, Fusarium heterosporum, Escherichia coli, Bacillus, preferably Bacillus subtilis, or Bacillus licheniformis, Pseudomonas, preferably Pseudomonas fluorescens, Pichia pastoris (also known as Komagataella phaffii), Myceliopthora thermophile (C1), Themothelomyces thermophila, Schizosaccharomyces pombe, Trichoderma, preferably Trichoderma reesei and Saccharomyces, preferably Saccharomyces cerevisiae. The variant polypeptides may be produced using the expression system listed above.

In one embodiment, the bacterial expression system is selected from an E. col, a Bacillus, a Pseudomonas, and a Streptomyces. In one embodiment, the yeast expression system is selected from a Candida, a Pichia, a Saccharomyces, a Schizosaccharomyces. In one embodiment, the fungal expression system is selected from a Penicillium, an Aspergillus, a Fusarium, a Myceliopthora, a Rhizomucor, a Rhizopus, a Thermomyces, and a Trichoderma.

The term “heterologous” (or exogenous or foreign or recombinant) in the context of polynucleotides and polypeptides is defined herein as:
(a) not native to the host cell;
(b) native to the host cell but structural modifications, e.g., deletions, substitutions, and/or insertions, are included as a result of manipulation of the DNA of the host cell by recombinant DNA techniques to alter the native sequence; or
(c) native to the host cell but expression is quantitatively altered or expression is directed from a genomic location different from the native host cell as a result of manipulation of the DNA of the host cell by recombinant DNA techniques, e.g., a stronger promoter.

With respect to two or more polynucleotide sequences or two or more amino acid sequences, the term “heterologous” is used to characterize that the two or more polynucleotide sequences or two or more amino acid sequences do not occur naturally in the specific combination with each other.

“Genetic Construct” or “expression cassette” as used herein, is a DNA molecule composed of at least one sequence of interest to be expressed, operably linked to one or more control sequences (at least to a promoter) as described herein. Typically, the expression cassette comprises three elements: a promoter sequence, an open reading frame, and a 3′ untranslated region that, in eukaryotes, usually contains a polyadenylation site. Additional regulatory elements may include transcriptional as well as translational enhancers. An intron sequence may also be added to the 5′ untranslated region (UTR) or in the coding sequence to increase the amount of the mature message that accumulates in the cytosol. The expression cassette may be part of a vector or may be integrated into the genome of a host cell and replicated together with the genome of its host cell. The expression cassette usually is capable of increasing or decreasing expression.

The term “vector” as used herein comprises any kind of construct suitable to carry foreign polynucleotide sequences for transfer to another cell, or for stable or transient expression within a given cell. The term “vector” as used herein encompasses any kind of cloning vehicles, such as but not limited to plasmids, phagemids, viral vectors (e.g., phages), bacteriophage, baculoviruses, cosmids, fosmids, artificial chromosomes, or and any other vectors specific for specific hosts of interest. Low copy number or high copy number vectors are also included. Foreign polynucleotide sequences usually comprise a coding sequence which may be referred to herein as “gene of interest”. The gene of interest may comprise introns and exons, depending on the kind of origin or destination of host cell.

A vector as used herein may provide segments for transcription and translation of a foreign polynucleotide upon transformation into a host cell or host cell organelles. Such additional segments may include regulatory nucleotide sequences, one or more origins of replication that is required for its maintenance and/or replication in a specific cell type, one or more selectable markers, a polyadenylation signal, a suitable site for the insertion of foreign coding sequences such as a multiple cloning site etc. One example is when a vector is required to be maintained in a bacterial cell as an episomal genetic element (e.g. plasmid or cosmid molecule). Non-limiting examples of suitable origins of replication include the f1-ori and colE1.

A vector may replicate without integrating into the genome of a host cell, e.g. as a plasmid in a bacterial host cell, or it may integrate part or all of its DNA into the genome of the host cell and thus lead to replication and expression of its DNA.

Foreign nucleic acid may be introduced into a vector by means of cloning. Cloning may mean that by cleavage of the vector (e.g. within the multiple cloning site) and the foreign polynucleotide by suitable means and methods (e.g., restriction enzymes), fitting structures within the individual nucleic acids may be created that enable the controlled fusion of said foreign nucleic acid and the vector.

Once introduced into the vector, the foreign nucleic acid comprising a coding sequence may be suitable to be introduced (transformed, transduced, transfected, etc.) into a host cell or host cell organelles. A cloning vector may be chosen suitable for expression of the foreign polynucleotide sequence in the host cell or host cell organelles.

The term “introduction” or “transformation” as referred to herein encompasses the transfer of an exogenous polynucleotide into a host cell, irrespective of the method used for transfer. That is, the term “transformation” as used herein is independent from vector, shuttle system, or host cell, and it not only relates to the polynucleotide transfer method of transformation as known in the art (cf., for example, Sambrook, J. et al. (1989) Molecular Cloning: A Laboratory Manual, 2nd Ed., Cold Spring Harbor Laboratory Press, Cold Spring Harbor, N.Y.), but it encompasses any further kind polynucleotide transfer methods such as, but not limited to, transduction or transfection. Plant tissue capable of subsequent clonal propagation, whether by organogenesis or embryogenesis, may be transformed with a genetic construct and a whole plant regenerated therefrom. The particular tissue chosen will vary depending on the clonal propagation systems available for, and best suited to, the particular species being transformed.

In one embodiment of the invention, a vector is used for transformation of a host cell.

The polynucleotide may be transiently or stably introduced into a host cell and may be maintained non-integrated, for example, as a plasmid. “Stable transformation” may mean that the transformed cell or cell organelle passes the nucleic acid comprising the foreign coding sequence on to the next generations of the cell or cell organelles. Usually stable transformation is due to integration of nucleic acid comprising a foreign coding sequence into the chromosomes or as an episome (separate piece of nuclear DNA).

“Transient transformation” may mean that the cell or cell organelle once transformed expresses the foreign nucleic acid sequence for a certain time—mostly within one generation. Usually transient transformation is due to nucleic acid comprising a foreign nucleic acid sequence is not integrated into the chromosomes or as an episome.

Alternatively, it may be integrated into the host genome. The resulting transformed plant cell may then be used to regenerate a transformed plant in a manner known to persons skilled in the art.

Recombinant cells may exhibit “increased” or “decreased” expression when compared to the respective wild-type cell.

The term “increased expression”, “enhanced expression” or “overexpression” as used herein means any form of expression that is additional to the original wild-type expression level (which can be absence of expression or immeasurable expression as well). Reference herein to “increased expression”, “enhanced expression” or “overexpression” is taken to mean an increase in gene expression and/or, as far as referring to polypeptides, increased polypeptide levels and/or increased polypeptide activity, relative to control organisms. The increase in expression may be in increasing order of preference at least 5%, at least 10%, at least 20%, at least 30%, at least 40%, at least 50%, at least 60%, at least 70%, at least 80%, at least 85%, at least 90%, or 100% or even more compared to that of control organisms.

Methods for increasing expression of genes or gene products are well documented in the art and include, for example, overexpression driven by appropriate promoters, the use of transcription enhancers or translation enhancers. Isolated nucleic acids which serve as promoter or enhancer elements may be introduced in an appropriate position (typically upstream) of a non-heterologous form of a polynucleotide so as to increase expression of a nucleic acid encoding the polypeptide of interest. For example, endogenous promoters may be altered in vivo by mutation, deletion, and/or substitution (see, Kmiec, U.S. Pat. No. 5,565,350; Zarling et al., WO 93/22443), or isolated promoters may be introduced into an organism in the proper orientation and distance from a gene of the present invention so as to control the expression of the gene.

An intron sequence may also be added to the 5′ untranslated region (UTR) or the coding sequence of the partial coding sequence to increase the amount of the mature message that accumulates in the cytosol. Inclusion of a spliceable intron in the transcription unit in expression constructs has been shown to increase gene expression at both the mRNA and protein levels up to 1000-fold (Buchman and Berg (1988) Mol. Cell biol. 8: 4395-4405; Callis et al. (1987) Genes Dev 1:1183-1200). Such intron enhancement of gene expression is typically greatest when placed near the 5′ end of the transcription unit.

To obtain increased expression or overexpression of a polypeptide most commonly the nucleic acid encoding this polypeptide is overexpressed in sense orientation with a polyadenylation signal. Introns or other enhancing elements may be used in addition to a promoter suitable for driving expression with the intended expression pattern.

Enzymes are generally produced commercially by using recombinant cells which express the desired enzyme by cultivation of the same under conditions suitable for expression of the desired enzyme.

Cultivation normally takes place in a suitable nutrient medium allowing the recombinant cells to grow (this process may be called fermentation) and express the desired protein. At the end of fermentation, fermentation broth is collected and may be further processed, wherein the fermentation broth comprises a liquid fraction and a solid fraction.

The enzyme of interest may be further purified from the fermentation broth. The term “purification” or “purifying” refers to a process in which at least one component, e.g., a protein of interest, is separated from at least another component, e.g., a particulate matter of a fermentation broth, and transferred into a different compartment or phase, wherein the different compartments or phases do not necessarily need to be separated by a physical barrier. Examples of such different compartments are two compartments separated by a filtration membrane or cloth, i.e., filtrate and retentate; examples of such different phases are pellet and supernatant or cake and filtrate, respectively. The resulting solution after purifying the enzyme of interest from the fermentation broth is called herein “purified enzyme solution”.

The desired enzyme may be secreted (into the liquid fraction of the fermentation broth) or may not be secreted from the host cells (and therefore is comprised in the cells of the fermentation broth). Depending on this, the desired enzyme may be recovered from the liquid fraction of the fermentation broth or from cell lysates. Recovery of the desired enzyme uses methods known to those skilled in the art. Suitable methods for recovery of proteins or enzymes from fermentation broth include but are not limited to collection, centrifugation, filtration, extraction, and precipitation. If the enzyme of interest precipitates or crystallizes in the fermentation broth or binds at least in part to the particulate matter of the fermentation broth additional treatment steps might be needed to release the enzyme from the biomass or solubilize enzyme crystals and precipitates. U.S. Pat. No. 6,316,240B1 describes a method for recovering an enzyme, which precipitates and/or crystallizes during fermentation, from the fermentation broth. In case the desired enzyme is comprised in the cells of the fermentation broth release of the enzyme from the cells might be needed. Release from the cells can be achieved for instance, but not being limited thereto, by cell lysis with techniques well known to the skilled person.

The purified enzyme solution may be further processed to form an “enzyme formulation”. “Enzyme formulation” means any non-complex formulation comprising a small number of ingredients, wherein the ingredients serve the purpose of stabilizing the enzymes comprised in the enzyme formulation and/or the stabilization of the enzyme formulation itself. The term “enzyme stability” relates to the retention of enzymatic activity as a function of time during storage or operation. The term “enzyme formulation stability” relates to the maintenance of physical appearance of the enzyme formulation during storage or operation as well as the avoidance of microbial contamination during storage or operation.

An “enzyme formulation” is a composition which is meant to be formulated into a complex formulation which itself may be determined for final use. An “enzyme formulation” according to the invention is not a complex formulation comprising several components, wherein the components are formulated into the complex formulation to exert each individually a specific action in a final application. A complex formulation may be without being limited thereto a detergent formulation, wherein individual detergent components are formulated in amounts effective in the washing performance of the detergent formulation.

In one aspect of the invention, at least one amylase variant of the invention is comprised in an enzyme formulation.

The enzyme formulation can be either solid or liquid. Enzyme formulations can be obtained by using techniques known in the art. For instance, without being limited thereto, solid enzyme formulations can be obtained by extrusion or granulation. Suitable extrusion and granulation techniques are known in the art and are described for instance in WO9419444A1 and WO9743482A1.

“Liquid” in the context of enzyme formulation is related to the physical appearance at 20° C. and 101.3 kPa.

Liquid enzyme formulations may comprise amounts of enzyme in the range of 0.1% to 40% by weight, or 0.5% to 30% by weight, or 1% to 25% by weight, or 3% to 10%, all relative to the total weight of the enzyme formulation.

The liquid enzyme formulation may comprise more than one type of enzyme. In one embodiment, the enzyme formulation comprises one or more amylases according to the present invention. In one embodiment, the enzyme formulation comprises one or more amylases according to the present invention and at least one additional enzyme selected from the group consisting of selected from the group consisting of: a second amylase, a lipase, a protease, a cellulase, a laccase, a pectinase, a nuclease, and any combination thereof.

Aqueous enzyme formulations of the invention may comprise water in amounts of more than about 50% by weight, more than about 60% by weight, more than about 70% by weight, or more than about 80% by weight, all relative to the total weight of the enzyme formulation.

Liquid enzyme formulations of the invention may comprise residual components such as salts originating from the fermentation medium, cell debris originating from the production host cells, metabolites produced by the production host cells during fermentation.

In one embodiment, residual components may be comprised in liquid enzyme formulations in amounts less than 30% by weight, less than 20% by weight less, than 10% by weight, or less than 5% by weight, all relative to the total weight of the aqueous enzyme formulation. In one embodiment, the enzyme formulation, in particular the liquid enzyme formulation, comprises in addition to the one or more enzymes one or more additional compounds selected from the group consisting of solvent, salt, pH regulator, preservative, stabilizer, chelators, and thickening agent. The preservative in a liquid enzyme formulation maybe a sorbitol, a benzoate, a proxel, or any combination therefore. The stabilizers in a liquid enzyme formulation maybe an MPG, a glycerol, an acetate, or any combination thereof. The chelators in a liquid enzyme formulation maybe a citrate.

In one embodiment, an enzyme formulation comprises at least one polypeptide variant of the invention and at least one preservative. Non-limiting examples of suitable preservatives include (quaternary) ammonium compounds, isothiazolinones, organic acids, and formaldehyde releasing agents. Non-limiting examples of suitable (quaternary) ammonium compounds include benzalkonium chlorides, polyhexamethylene biguanide (PHMB), Didecyldimethylammonium chloride (DDAC), and N-(3-aminopropyl)-N-dodecylpropane-1,3-diamine (Diamine). Non-limiting examples of suitable isothiazolinones include 1,2-benzisothiazolin-3-one (BIT), 2-methyl-2H-isothiazol-3-one (MIT), 5-chloro-2-methyl-2H-isothiazol-3-one (CIT), 2-octyl-2H-isothiazol-3-one (OIT), and 2-butyl-benzo[d]isothiazol-3-one (BBIT). Non-limiting examples of suitable organic acids include benzoic acid, sorbic acid, L-(+)-lactic acid, formic acid, and salicylic acid. Non-limiting examples of suitable formaldehyde releasing agent include N,N′-methylenebismorpholine (MBM), 2,2′,2″-(hexahydro-1,3,5-triazine-1,3,5-triyl)triethanol (HHT), (ethylenedioxy)dimethanol, .alpha.,.alpha.′,.alpha.″-trimethyl-1,3,5-triazine-1,3,5(2H,4H,6H)-triethanol (HPT), 3,3′-methylenebis[5-methyloxazolidine] (MBO), and cis-1-(3-chloroallyl)-3,5,7-triaza-1-azoniaadamantane chloride (CTAC).

Further useful preservatives include iodopropynyl butylcarbamate (IPBC), halogen releasing compounds such as dichloro-dimethyl-hydantoine (DCDMH), bromo-chloro-dimethyl-hydantoine (BCDMH), and dibromo-dimethyl-hydantoine (DBDMH); bromo-nitro compounds such as Bronopol (2-bromo-2-nitropropane-1,3-diol), 2,2-dibromo-2-cyanoacetamide (DBNPA); aldehydes such as glutaraldehyde; phenoxyethanol; Biphenyl-2-ol; and zinc or sodium pyrithione.

In one embodiment, an enzyme formulation comprises at least one polypeptide variant of the invention and at least one enzyme stabilizer. An enzyme stabilizer is selected from substances which are capable of reducing loss of enzymatic activity during storage of at least one enzyme comprised in a liquid enzyme formulation. Reduced loss of enzymatic activity within this invention may mean that the loss of enzymatic activity is reduced by at least 5%, by at least 10%, by at least 15%, by at least 20%, by at least 25%, by at least 30%, by at least 40%, by at least 50%, by least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or at least 99.5% when compared to the initial enzymatic activity before storage. Preferred stabilizers are selected from the group consisting of salt (e.g., CaCl2), propanediol, polyethylene glycol, an MPG, a glycerol, an acetate, or any combination thereof.

Enzyme Applications

In another embodiment, the polypeptide variant as described herein may be used in foods, for example the enzyme can be an additive for baking. The enzymes can be used in feed, for example the enzyme is an animal feed additive. The enzyme can be used in the starch processing industry, for example the amylases are used in the conversion of starch to ethanol or sugars (high fructose corn syrup) and other byproducts such as oil, dry distiller's grains, etc. The polypeptide variants are used in in pulp and paper processing, for example, the enzymes can be used for improving paper strength. The enzymes can be used for mining and oil well services, for example cellulases can be used for breaking guar during oil well fracturing. In one embodiment, the polypeptide variant as described herein are used in detergent formulations or cleaning formulations.

In one embodiment, the present invention refers to a method of preparing a dough or a baked product prepared from the dough, the method comprising adding one of the variant polypeptides having amylase activity as described herein to the dough and baking it. In one embodiment, the present invention refers to a method of use of the variant polypeptide having amylase activity as described herein for processing starch. In one embodiment, the present invention refers to a method of use of the variant polypeptide having amylase activity as described herein for cleaning or washing textiles, hard surfaces, or dishes. In one embodiment, the present invention refers to a method of use of the variant polypeptide having amylase activity as described herein for making ethanol. In one embodiment, the present invention refers to a method of use of the variant polypeptide having amylase activity as described herein for processing pulp or paper. In one embodiment, the present invention refers to a method of use of the variant polypeptide having amylase activity as described herein for feeding an animal.

In one embodiment, the amylases of the present invention are used in detergent formulations or cleaning formulations.

“Detergent formulation” or “cleaning formulation” means compositions designated for cleaning soiled material. Cleaning includes laundering and hard surface cleaning. Soiled material according to the invention includes textiles and/or hard surfaces.

The term “laundering” relates to both household laundering and industrial laundering and means the process of treating textiles with a solution containing a detergent composition of the present invention. The laundering process may be carried out by using technical devices such as a household or an industrial washing machine. Alternatively, the laundering process may be done by hand.

The term “textile” means any textile material including yarns (thread made of natural or synthetic fibers used for knitting or weaving), yarn intermediates, fibers, non-woven materials, natural materials, synthetic materials, as well as fabrics (a textile made by weaving, knitting or felting fibers) made of these materials such as garments (any article of clothing made of textile), cloths and other articles.

The term “fibers” includes natural fibers, synthetic fibers, and mixtures thereof. Examples of natural fibers are of plant (such as flax, jute and cotton) or animal origin, comprising proteins like collagen, keratin and fibroin (e.g. silk, sheep wool, angora, mohair, cashmere). Examples for fibers of synthetic origin are polyurethane fibers such as Spandex® or Lycra®, polyester fibers, polyolefins such as elastofin, or polyamide fibers such as nylon. Fibers may be single fibers or parts of textiles such as knitwear, wovens, or nonwovens.

The term “hard surface cleaning” is defined herein as cleaning of hard surfaces wherein hard surfaces may include any hard surfaces in the household, such as floors, furnishing, walls, sanitary ceramics, glass, metallic surfaces including cutlery or dishes.

The term “dish wash” refers to all forms of washing dishes, e.g. by hand or automatic dish wash. Dish washing includes, but is not limited to, the cleaning of all forms of crockery such as plates, cups, glasses, bowls, all forms of cutlery such as spoons, knives, forks and serving utensils as well as ceramics, plastics such as melamine, metals, china, glass and acrylics.

The detergent formulation of the invention comprises one or more detergent component(s). The component(s) chosen depend(s) on the desired cleaning application and/or physical form of a detergent composition.

The term “detergent component” is defined herein to mean any types of ingredient, which is suitable for detergent compositions, such as surfactants, building agents, polymers, bleaching systems. Any component(s) known in the art acknowledging their known characteristics are suitable detergent component(s) according to the invention. Detergent components in one embodiment means components which provide washing or cleaning performance or which effectively aid the processing (maintain physical characteristics during processing, storage and use; e.g. rheology modifiers, hydrotropes, desiccants) when present in effective amounts.

Usually, a detergent composition is a complex formulation of more than two detergent components.

Detergent components may have more than one function in the final application of a detergent formulation, therefore any detergent component mentioned in the context of a specific function herein, may also have another function in the final application of a detergent formulation. The function of a specific detergent component in the final application of a detergent formulation usually depends on its amount within the detergent formulation, i.e. the effective amount of a detergent component.

The term “effective amount” includes amounts of certain components to provide effective stain removal and effective cleaning conditions (e.g. pH, quantity of foaming), amounts of certain components to effectively provide optical benefits (e.g. optical brightening, dye transfer inhibition), and amounts of certain components to effectively aid the processing (maintain physical characteristics during processing, storage and use; e.g. rheology modifiers, hydrotropes, desiccants).

In one embodiment, a detergent formulation is a formulation of more than two detergent components, wherein at least one component is effective in stain-removal, at least one component is effective in providing the optimal cleaning conditions, and at least one component is effective in maintaining the physical characteristics of the detergent.

Cleaning performance is evaluated under relevant cleaning conditions. The term “relevant cleaning conditions” herein refers to the conditions, particularly cleaning temperature, time, cleaning mechanics, suds concentration, type of detergent and water hardness, actually used in laundry machines, automatic dish washers or in manual cleaning processes.

Individual detergent components and usage in detergent compositions are known to those skilled in the art. Suitable detergent components comprise inter alia surfactants, builders, polymers, alkaline, bleaching systems, fluorescent whitening agents, suds suppressors and stabilizers, hydrotropes, and corrosion inhibitors. Further examples are described e.g. in “complete Technology Book on Detergents with Formulations (Detergent Cake, Dishwashing Detergents, Liquid & Paste Detergents, Enzyme Detergents, Cleaning Powder & Spray Dried Washing Powder)”, Engineers India Research Institute (EIRI), 6^{th} edition (2015). Another reference book for those skilled in the art may be “Detergent Formulations Encyclopedia”, Solverchem Publications, 2016.

Detergent components vary in type and/or amount in a detergent formulation depending on the desired application such as laundering white textiles, colored textiles, and wool. The component(s) chosen further depend(s) on physical form of a detergent formulation (liquid, solid, gel, provided in pouches or as a tablet, etc). The component(s) chosen e.g. for laundering formulations further depend on regional conventions which themselves are related to aspects like washing temperatures used, mechanics of laundry machine (vertical vs. horizontal axis machines), water consumption per wash cycle etc. and geographical characteristics like average hardness of water.

For example: A low detergent concentration system includes laundering formulations where less than about 800 ppm of detergent components are present in the wash water. A medium detergent concentration includes laundering formulations where between about 800 ppm and about 2,000 ppm of detergent components are present in the wash water. A high detergent concentration includes laundering formulations where more than about 2,000 ppm of detergent components are present in the wash water.

The numeric ranges recited for the individual detergent components provide amounts comprised in detergent compositions. Such ranges have to be understood to be inclusive of the numbers defining the range and include each integer within the defined range.

If not described otherwise, “% by weight” or “% w/w” is meant to be related to total detergent composition. In this case “% by weight” or “% w/w” is calculated as follows: concentration of a substance as the weight of that substance divided by the total weight of the composition, multiplied by 100.

Detergent formulations of the invention may comprise one or more surfactant(s). “Surfactant” (synonymously used herein with “surface active agent”) means an organic chemical that, when added to a liquid, changes the properties of that liquid at an interface. According to its ionic charge, a surfactant is called non-ionic, anionic, cationic, or amphoteric.

Non-limiting examples of surfactants are disclosed McCutcheon's 2016 Detergents and Emulsifiers, and McCutcheon's 2016 Functional Materials, both North American and International Edition, MC Publishing Co, 2016 edition. Further useful examples are disclosed in earlier editions of the same publications which are known to those skilled in the art.

Non-ionic surfactant means a surfactant that contains neither positively nor negatively charged (i.e. ionic) functional groups. In contrast to anionic and cationic surfactants, non-ionic surfactants do not ionize in solution.

Examples provided below for surfactants of any kind are to be understood to be non-limiting.

Non-ionic surfactants may be compounds of the general formulae (Ia) and (Ib):

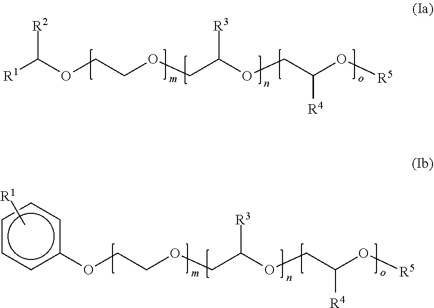

The variables of the general formulae (Ia) and (Ib) are defined as follows:
R¹ is selected from C₁-C₂₃ alkyl and C₂-C₂₃ alkenyl, wherein alkyl and/or alkenyl are linear or branched; examples are n-C₇H₁₅, n-C₉H₁₉, n-C₁₁H₂₃, n-C₁₃H₂₇, n-C₁₅H₃₁, nC₁₇H₃₅, i-C₉H₁₉, i-C₁₂H₂₅.
R² is selected from H, C₁-C₂₀ alkyl and C₂-C₂₀ alkenyl, wherein alkyl and/or alkenyl are linear or branched.
R³ and R⁴, each independently selected from C₁-C₁₆ alkyl, wherein alkyl is linear or branched; examples are methyl, ethyl, n-propyl, isopropyl, n-butyl, isobutyl, sec-butyl, tert-butyl, n-pentyl, isopentyl, sec-pentyl, neopentyl, 1,2-dimethylpropyl, isoamyl, n-hexyl, isohexyl, sec-hexyl, n-heptyl, n-octyl, 2-ethylhexyl, n-nonyl, n-decyl, isodecyl.
R⁵ is selected from H and C₁-C₁₈ alkyl, wherein alkyl is linear or branched.

The integers of the general formulae (Ia) and (Ib) are defined as follows:

m is in the range of zero to 200, preferably 1-80, more preferably 3-20; n and o, each independently in the range of zero to 100; n preferably is in the range of 1 to 10, more preferably 1 to 6; o preferably is in the range of 1 to 50, more preferably 4 to 25. The sum of m, n and o is at least one, preferably the sum of m, n and o is in the range of 5 to 100, more preferably in the range of from 9 to 50.

The non-ionic surfactants of the general formula (Ia) and (Ib) may be of any structure, is it block or random structure, and is not limited to the displayed sequences.

In one embodiment, the detergent formulation comprises at least one non-ionic surfactant selected from general formula (Ia), wherein m is in the range of 3 to 11, preferably not more than 7; n and o is 0, R¹ is C₁₂-C₁₄, R⁵ is H. The detergent formulation may comprise at least two non-ionic surfactant, selected from compounds of general formula (Ia), wherein one of said non-ionic surfactants is characterized in R¹ being C₁₂, R⁵ being H, m is 7, n and o=0, and the other surfactant is characterized in R¹ being C₁₄, R⁵ being H, m being 7, n and o=0.

Non-ionic surfactants may further be compounds of the general formula (II), which might be called alkyl-polyglycosides (APG):

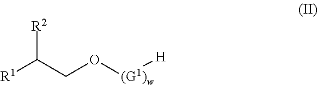

The variables of the general formula (II) are defined as follows:
R¹ is selected from C₁-C₁₇ alkyl and C₂-C₁₇ alkenyl, wherein alkyl and/or alkenyl are linear or branched; examples are n-C₇H₁₅, n-C₉H₁₉, n-C₁₁H₂₃, n-C₁₃H₂₇, n-C₁₅H₃₁, n-C₁₇H₃₅, i-C₉H₁₉, i-C₁₂H₂₅.
R² is selected from H, C₁-C₁₇ alkyl and C₂-C₁₇ alkenyl, wherein alkyl and/or alkenyl are linear or branched.
G¹ is selected from monosaccharide residues with 4 to 6 carbon atoms, such as glucose and xylose.

The integer w of the general formula (II) is in the range of from 1.1 to 4, w being an average number.

Non-ionic surfactants may further be compounds of general formula (III):

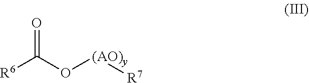

The variables of the general formula (III) are defined as follows:
AO is selected from ethylene oxide (EO), propylene oxide (PO), butylene oxide (BO), and mixtures thereof.
R⁶ is selected from C₅-C₁₇ alkyl and C₅-C₁₇ alkenyl, wherein alkyl and/or alkenyl are linear or branched.
R⁷ is selected from H, C₁-C₁₈-alkyl, wherein alkyl is linear or branched.

The integer y of the general formula (III) is a number in the range of 1 to 70, preferably 7 to 15.

Non-ionic surfactants may further be selected from sorbitan esters and/or ethoxylated or propoxylated sorbitan esters. Non-limiting examples are products sold under the trade names SPAN and TWEEN.

Non-ionic surfactants may further be selected from alkoxylated mono- or di-alkylamines, fatty acid monoethanolamides (FAMA), fatty acid diethanolamides (FADA), ethoxylated fatty acid monoethanolamides (EFAM), propoxylated fatty acid monoethanolamides (PFAM), polyhydroxy alkyl fatty acid amides, or N-acyl N-alkyl derivatives of glucosamine (glucamides, GA, or fatty acid glucamide, FAGA), and combinations thereof.

Mixtures of two or more different non-ionic surfactants may also be present in detergent formulations according to the present invention.

Amphoteric surfactants are those, depending on pH, which can be either cationic, zwitterionic or anionic.

Surfactants may be compounds comprising amphoteric structures of general formula (IV), which might be called modified amino acids (proteinogenic as well as non-proteinogenic):

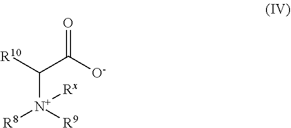

The variables in general formula (IV) are defined as follows:
R⁸ is selected from H, C₁-C₄ alkyl, C₂-C₄ alkenyl, wherein alkyl and/or are linear or branched.
R⁹ is selected from C₁-C₂₂ alkyl, C₂-C₂₂ alkenyl, C₁₀-C₂₂ alkylcarbonyl, and C₁₀-C₂₂ alkenylcarbonyl.
R¹⁰ is selected from H, methyl, —(CH₂)₃NHC(NH)NH₂, —CH₂C(O)NH₂, —CH₂C(O)OH, —(CH₂)₂C(O)NH₂, —(CH₂)₂C(O)OH, (imidazole-4-yl)-methyl, —CH(CH₃)C₂H₅, —CH₂CH(CH₃)₂, —(CH₂)₄NH₂, benzyl, hydroxymethyl, —CH(OH)CH₃, (indole-3-yl)-methyl, (4-hydroxy-phenyl)methyl, isopropyl, —(CH₂)₂SCH₃, and —CH₂SH.
R^{x} is selected from H and C₁-C₄ alkyl.

Surfactants may further be compounds comprising amphoteric structures of general formulae (Va), (Vb), or (Vc), which might be called betaines and/or sulfobetaines:

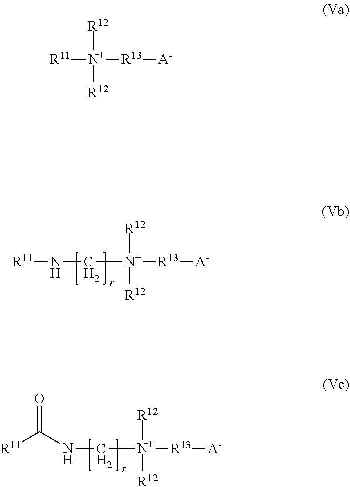

The variables in general formulae (Va), (Vb) and (Vc) are defined as follows:
R¹¹ is selected from linear or branched C₁₀-C₂₂ alkyl and linear or branched C₁₀-C₂₂ alkenyl.
R¹² are each independently selected from linear C₁-C₄ alkyl.
R¹³ is selected from C₁-C₅ alkyl and hydroxy C₁-C₅ alkyl; for example, 2-hydroxypropyl.
A⁻ is selected from carboxylate and sulfonate.

The integer r in general formulae (Va), (Vb), and (Vc) is in the range of 2 to 6.

Surfactants may further be compounds comprising amphoteric structures of general formula (VI), which might be called alkyl-amphocarboxylates:

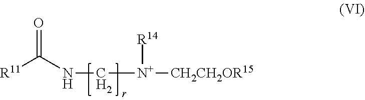

The variables in general formula (VI) are defined as follows:
R¹¹ is selected from C₇-C₂₂ alkyl and C₇-C₂₂ alkenyl, wherein alkyl and/or alkenyl are linear or branched, preferably linear.
R¹⁴ is selected from —CH₂C(O)O⁻M⁺, —CH₂CH₂C(O)O⁻M⁺ and —CH₂CH(OH)CH₂SO₃⁻M⁺.
R¹⁵ is selected from H and —CH₂C(O)O⁻.

The integer r in general formula (VI) is in the range of 2 to 6.

Non-limiting examples of further suitable alkyl-amphocarboxylates include sodium cocoamphoacetate, sodium lauroamphoacetate, sodium capryloamphoacetate, disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, and disodium capryloamphodipropionate.

Surfactants may further be compounds comprising amphoteric structures of general formula (VII), which might be called amine oxides (AO):

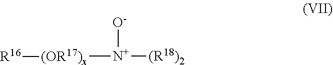

The variables in general formula (VII) are defined as follows:
R¹⁶ is selected from C₈-C₁₈ linear or branched alkyl, hydroxy C₈-C₁₈ alkyl, acylamidopropoyl and C₈-C₁₈ alkyl phenyl group; wherein alkyl and/or alkenyl are linear or branched.
R¹⁷ is selected from C₂-C₃ alkylene, hydroxy C₂-C₃ alkylene, and mixtures thereof.
R¹⁸ each residue can be independently selected from C₁-C₃ alkyl and hydroxy C₁-C₃; R¹⁸ groups can be attached to each other, e.g., through an oxygen or nitrogen atom, to form a ring structure.

The integer x in general formula (VII) is in the range of 0 to 5, preferably from 0 to 3, most preferably 0.

Non-limiting examples of further suitable amine oxides include C₁₀-C₁₈ alkyl dimethyl amine oxides and C₈-C₁₈ alkoxy ethyl dihydroxyethyl amine oxides. Examples of such materials include dimethyloctyl amine oxide, diethyldecyl amine oxide, bis-(2-hydroxyethyl) dodecyl amine oxide, dimethyldodecylamine oxide, dipropyltetradecyl amine oxide, methylethylhexadecyl amine oxide, dodecylamidopropyl dimethyl amine oxide, cetyl dimethyl amine oxide, stearyl dimethyl amine oxide, tallow dimethyl amine oxide and dimethyl-2-hydroxyoctadecyl amine oxide.

A further example of a suitable amine oxide is cocamidylpropyl dimethylaminoxide, sometimes also called cocamidopropylamine oxide.

Mixtures of two or more different amphoteric surfactants may be present in detergent compositions according to the present invention.

Anionic surfactant means a surfactant with a negatively charged ionic group. Anionic surfactants include, but are not limited to, surface-active compounds that contain a hydrophobic group and at least one water-solubilizing anionic group, usually selected from sulfates, sulfonate, and carboxylates to form a water-soluble compound.

Anionic surfactants may be compounds of general formula (VIII), which might be called (fatty) alcohol/alkyl (ethoxy/ether) sulfates [(F)A(E)S] when A⁻ is SO₃⁻, (fatty) alcohol/alkyl (ethoxy/ether) carboxylate [(F)A(E)C] when A⁻ is —RCOO-:

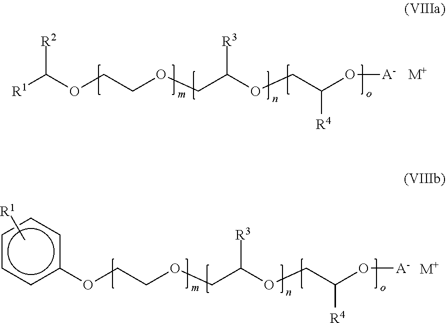

The variables in genera formulae (VIIIa and VIIIb) are defined as follows:

R¹ is selected from C₁-C₂₃-alkyl (such as 1-, 2-, 3-, 4-C₁-C₂₃-alkyl) and C₂-C₂₃-alkenyl, wherein alkyl and/or alkenyl are linear or branched, and wherein 2-, 3-, or 4-alkyl; examples are n-C₇H₁₅, n-C₉H₁₉, n-C₁₁H₂₃, n-C₁₃H₂₇, n-C₁₅H₃₁, n-C₁₇H₃₅, i-CH₁₉, i-C₁₂H₂₅.

R² is selected from H, C₁-C₂₀-alkyl and C₂-C₂₀-alkenyl, wherein alkyl and/or alkenyl are linear or branched.

R³ and R⁴, each independently selected from C₁-C₁₆-alkyl, wherein alkyl is linear or branched; examples are methyl, ethyl, n-propyl, isopropyl, n-butyl, isobutyl, sec-butyl, tert-butyl, n-pentyl, isopentyl, sec-pentyl, neopentyl, 1,2-dimethylpropyl, isoamyl, n-hexyl, isohexyl, sec-hexyl, n-heptyl, n-octyl, 2-ethylhexyl, n-nonyl, n-decyl, isodecyl.
A⁻ is selected from —RCOO—, —SO₃ and RSO₃-, wherein R is selected from linear or branched C₁-C₅-alkyl, and C₁-C₄ hydroxyalkyl, wherein alkyl is.
M⁺ is selected from H and salt forming cations. Salt forming cations may be monovalent or multivalent; hence M⁺ equals 1/v M^{v+}. Examples include but are not limited to sodium, potassium, magnesium, calcium, ammonium, and the ammonium salt of mono-, di, and triethanolamine.

The integers of the general formulae (VIIIa) and (VIIIb) are defined as follows:
m is in the range of zero to 200, preferably 1-80, more preferably 3-20; n and o, each independently in the range of zero to 100; n preferably is in the range of 1 to 10, more preferably 1 to 6; o preferably is in the range of 1 to 50, more preferably 4 to 25. The sum of m, n and o is at least one, preferably the sum of m, n and o is in the range of 5 to 100, more preferably in the range of from 9 to 50.

Anionic surfactants of the general formula (VIIIa) and (VIIIb) may be of any structure, block copolymers or random copolymers.

Further suitable anionic surfactants include salts (M⁺) of C₁₂-C₁₈ sulfo fatty acid alkyl esters (such as C₁₂-C₁₈ sulfo fatty acid methyl esters), C₁₀-C₁₈-alkylarylsulfonic acids (such as n-C₁₀-C₁₈-alkylbenzene sulfonic acids) and C₁₀-C₁₈ alkyl alkoxy carboxylates.

M⁺ in all cases is selected from salt forming cations. Salt forming cations may be monovalent or multivalent; hence M⁺ equals 1/v M^{v+}. Examples include but are not limited to sodium, potassium, magnesium, calcium, ammonium, and the ammonium salt of mono-, di, and triethanolamine.

In one embodiment, the detergent formulation comprises at least one anionic surfactant selected from compounds of general formula (VIIIa), wherein R¹ is C₁₁-C₁₃, R₂ is H, m is 1-4, n and o=0, A⁻ is SO₃⁻, M⁺is Na⁺. The detergent formulation may comprise at least two anionic surfactants, selected from compounds of general formula (VIIIa), wherein one of said anionic surfactants is characterized in R¹ being C₁₁, R² being H, m being 2, n and o=0, A⁻ being SO₃⁻, M⁺ being Na⁺ and the other surfactant is characterized in R¹ being C₁₃, R² being H, m being 2, n and o=0, A⁻ being SO₃⁻, M⁺ being Na⁺.

Non-limiting examples of further suitable anionic surfactants include branched alkylbenzenesulfonates (BABS), phenylalkanesulfonates, alpha-olefinsulfonates (AOS), olefin sulfonates, alkene sulfonates, alkane-2,3-diylbis(sulfates), hydroxyalkanesulfonates and disulfonates, secondary alkanesulfonates (SAS), paraffin sulfonates (PS), sulfonated fatty acid glycerol esters, alkyl- or alkenylsuccinic acid, fatty acid derivatives of amino acids, diesters and monoesters of sulfo-succinic acid.

In one embodiment, the detergent formulation comprises at least one anionic surfactant selected from compounds of general formula (IX):

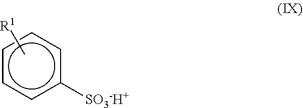

wherein R¹ in formula (IX) is C₁₀-C₁₃ alkyl. The detergent formulation may comprise at least two anionic surfactants, selected from compounds of general formula (IX), wherein one of said anionic surfactants is characterized in R¹ being C₁₀, and the other surfactant is characterized in R¹ being C₁₃.

Anionic surfactants may be compounds of general formula (X), which might be called N-acyl amino acid surfactants:

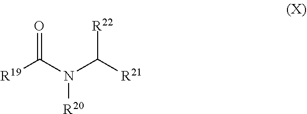

The variables in general formula (X) are defined as follows:
R¹⁹ is selected from linear or branched C₆-C₂₂-alkyl and linear or branched C₆-C₂₂-alkenyl such as oleyl.
R²⁰ is selected from H and C₁-C₄-alkyl.
R²¹ is selected from H, methyl, —(CH₂)₃NHC(NH)NH₂, —CH₂C(O)NH₂, —CH₂C(O)OH, —(CH₂)₂C(O)NH₂, —(CH₂)₂C(O)OH, (imidazole-4-yl)-methyl, —CH(CH₃)C₂H₅, —CH₂CH(CH₃)₂, —(CH₂)₄NH₂, benzyl, hydroxymethyl, —CH(OH)CH₃, (indole-3-yl)-methyl, (4-hydroxy-phenyl)methyl, isopropyl, —(CH₂)₂SCH₃, and —CH₂SH.
R²² is selected from —COOX and —CH₂SO₃X, wherein X is selected from Li⁺, Na⁺ and K⁺.

Non-limiting examples of suitable N-acyl amino acid surfactants are the mono- and dicarboxylate salts (e.g., sodium, potassium, ammonium and ammonium salt of mono-, di, and triethanolamine) of N-acylated glutamic acid, for example, sodium cocoyl glutamate, sodium lauroyl glutamate, sodium myristoyl glutamate, sodium palmitoyl glutamate, sodium stearoyl glutamate, disodium cocoyl glutamate, disodium stearoyl glutamate, potassium cocoyl glutamate, potassium lauroyl glutamate, and potassium myristoyl glutamate; the carboxylate salts (e.g., sodium, potassium, ammonium and ammonium salt of mono-, di, and triethanolamine) of N-acylated alanine, for example, sodium cocoyl alaninate, and triethanolamine lauroyl alaninate; the carboxylate salts (e.g., sodium, potassium, ammonium and ammonium salt of mono-, di, and triethanolamine) of N-acylated glycine, for example, sodium cocoyl glycinate, and potassium cocoyl glycinate; the carboxylate salts (e.g., sodium, potassium, ammonium and ammonium salt of mono-, di, and triethanolamine) of N-acylated sarcosine, for example, sodium lauroyl sarcosinate, sodium cocoyl sarcosinate, sodium myristoyl sarcosinate, sodium oleoyl sarcosinate, and ammonium lauroyl sarcosinate.

Anionic surfactants may further be selected from the group of soaps. Suitable are salts (M⁺) of saturated and unsaturated C₁₂-C₁₈ fatty acids, such as lauric acid, myristic acid, palmitic acid, stearic acid, behenic acid, oleic acid, (hydrated) erucic acid. M⁺is selected from salt forming cations. Salt forming cations may be monovalent or multivalent; hence M⁺ equals 1/v M^{v+}. Examples include but are not limited to sodium, potassium, magnesium, calcium, ammonium, and the ammonium salt of mono-, di, and triethanolamine.

Further non-limiting examples of suitable soaps include soap mixtures derived from natural fatty acids such as tallow, coconut oil, palm kernel oil, laurel oil, olive oil, or canola oil. Such soap mixtures comprise soaps of lauric acid and/or myristic acid and/or palmitic acid and/or stearic acid and/or oleic acid and/or linoleic acid in different amounts, depending on the natural fatty acids from which the soaps are derived.

Further non-limiting examples of suitable anionic surfactants include salts (M⁺) of sulfates, sulfonates or carboxylates derived from natural fatty acids such as tallow, coconut oil, palm kernel oil, laurel oil, olive oil, or canola oil. Such anionic surfactants comprise sulfates, sulfonates or carboxylates of lauric acid and/or myristic acid and/or palmitic acid and/or stearic acid and/or oleic acid and/or linoleic acid in different amounts, depending on the natural fatty acids from which the soaps are derived.

Mixtures of two or more different anionic surfactants may also be present in detergent compositions according to the present invention.

Mixtures of non-ionic and/or amphoteric and/or anionic surfactants may also be present in detergent compositions according to the present invention.

Cationic surfactant means a surfactant with a positively charged ionic group.

Typically, these cationic moieties are nitrogen containing groups such as quaternary ammonium or protonated amino groups. The cationic protonated amines can be primary, secondary, or tertiary amines.

Cationic surfactants may be compounds of the general formula (XI) which might be called quaternary ammonium compounds (quats):

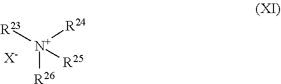

The variables in general formula (XI) are defined as follows:
R²³ is selected from H, C₁-C₄ alkyl (such as methyl) and C₂-C₄ alkenyl, wherein alkyl and/or alkenyl is linear or branched.
R²⁴ is selected from C₁-C₄ alkyl (such as methyl), C₂-C₄ alkenyl and C₁-C₄ hydroxyalkyl (such as hydroxyethyl), wherein alkyl and/or alkenyl is linear or branched.
R²⁵ is selected from C₁-C₂₂ alkyl (such as methyl, C₁, alkyl), C₂-C₄ alkenyl, C₁₂-C₂₂ alkylcarbonyloxymethyl and C₁₂-C₂₂ alkylcarbonyloxyethyl (such as C₁₆-C₁₈ alkylcarbonyloxyethyl), wherein alkyl and/or alkenyl is linear or branched.
R²⁶ is selected from C₁₂-C₁₈ alkyl, C₂-C₄ alkenyl, C₁₂-C₂₂ alkylcarbonyloxymethyl, C₁₂-C₂₂ alkylcarbonyloxyethyl and 3-(C₁₂-C₂₂ alkylcarbonyloxy)-2(C₁₂-C₂₂ alkylcarbonyloxy)-propyl.
X⁻ is selected from halogenid, such as Cl⁻ or Br⁻.

Non-limiting examples of further cationic surfactants include, amines such as primary, secondary and tertiary monoamines with C₁ alkyl or alkenyl chains, ethoxylated alkylamines, alkoxylates of ethylenediamine, imidazoles (such as 1-(2-hydroxyethyl)-2-imidazoline, 2-alkyl-1-(2-hydroxyethyl)-2-imidazoline, and the like), quaternary ammonium salts like alkylquaternary ammonium chloride surfactants such as n-alkyl(C₁₂-C₁₈)dimethylbenzyl ammonium chloride, n-tetradecyldimethylbenzylammonium chloride monohydrate, and a naphthylene-substituted quaternary ammonium chloride such as dimethyl-1-naphthylmethylammonium chloride.

Particularly suitable cationic surfactants that may be:
N,N-dimethyl-N-(hydroxy-C₇-C₂₅-alkyl)ammonium salts;
mono- and di(C₇-C₂₅-alkyl) dimethylammonium compounds quaternized with alkylating agents;
ester quats, in particular quaternary esterified mono-, di- and trialkanolamines which are esterified with C₁₀-C₂₂-carboxylic acids;
imidazoline quats, in particular 1-alkylimidazolinium salts of formulae XII or XIII

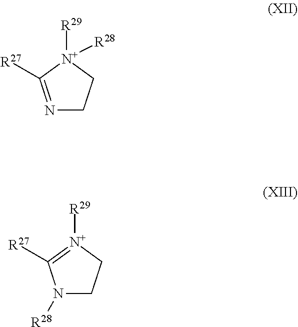

The variables in formulae (XII) and (XIII) are defined as follows:
R²⁷ is selected from C₁-C₂₅-alkyl and C₂-C₂₅-alkenyl;
R²⁸ is selected from C₁-C₄-alkyl and hydroxy-C₁-C₄-alkyl;
R²⁹ is selected from C₁-C₄-alkyl, hydroxy-C₁-C₄-alkyl and a R*—(CO)—R³⁰—(CH₂)j- radical, wherein R* is selected from C₁-C₂₁-alkyl and C₂-C₂₁-alkenyl; R³⁰ is selected from —O— and —NH—; j is 2 or 3.

The detergent formulation may comprise a mixture of surfactants selected from compounds of general formula (Ia), compounds of general formula (VIIIa), and compounds of general formula (IX).

### Example 1: Red Starch Assay

Quantitation of starch hydrolysis for the alpha-amylase and variant enzymes was measured using the Red Starch method as described by Megazyme, “Assay of Alpha-Amylase using RedStarch” with the following modifications. 10 μL of 2% red starch prepared in 100 mM HEPES, pH 8.0 buffer was reacted with 20 μL diluted enzyme at 25° C. The reaction was terminated after 10 min by the addition of 50 μL of 95% v/v ethanol. After vigorous mixing the reaction was equilibrated for 10 min at room temperature followed by centrifugation at 1,000×g for 8 min. 60 μL of the reaction was transferred and the solution absorption was read at 510 nm in a BioTek plate reader.

### Example 2: Residual Activity

Residual activity was calculated by comparing the activity of each enzyme as measured using the red starch assay before and after a heat challenge at 50 degrees C. After heating the sample for 15 minutes at 50 degrees C., the sample was chilled at 4° C. for 10 min before being tested, using the red starch assay, at 25° C.

### Example 3: ThermoFluor Assay

The melting point (Tm) for each alpha-amylase enzyme variant was measured in a high throughput manner using the ThermoFluor assay as described in Lo, M C; Aulabaugh, A; Jin, G; Cowling, R; Bard, J; Malamas, M; Ellestad, G (1 Sep. 2004). “Evaluation of fluorescence based thermal shift assays for hit identification in drug discovery” Analytical Biochemistry 332 (1): 153-9.10.1016/j.ab.2004.04.031, PMID 15301960, with the following modifications. A 20 μl reaction in 100 mM HEPES pH 8.0, 3×SYPRO orange, and enzyme supernatant was subjected to a heat ramp from 25° C. to 80° C. with a ramp rate of 1° C. per 30 sec. The dye fluorescence was monitored using a BioRad CFX 384 real-time PCR machine. The melt curve data was analyzed using the supplied CFX Manager Software and an internally developed melting temperature curation program.

### Example 4: pNP-G7 Assay

Amylase activity was measured by following the hydrolytic release of p-nitrophenol from a proprietary reagent (Infinity Amylase Liquid Stable Reagent, ThermoScientific) consisting of ethylidene-para-nitrophenyl-heptasaccharide (E-pNP-G7) and alpha-glucosidase. By linking a polysaccharide (glucose oligomer) to p-nitrophenol, it is possible to follow alpha-amylase activity of the target variant by detecting the liberation of p-nitrophenol at 405 nm. Amylases were expressed and the secreted amylase separated from the cells by centrifugation. The supernatants were added to the assay buffer and Infinity Amylase Liquid Stable Reagent in 96-well plate format. The absorbance at 405 nm was detected every 30 seconds over 30 minutes. The linear portion of the slope was used to determine the rate of activity.

### Example 5: Amylase Variant Expression

The variant polypeptides having alpha-amylase activity were obtained by constructing expression plasmids containing the encoding polynucleotide sequences, transforming plasmids into Pichia pastoris (Komagataella phaffii) and growing the resulting expression strains in the following way. Fresh Pichia pastoris cells of the expression strains were obtained by spreading the glycerol stocks of sequence-confirmed strains onto Yeast extract Peptone Dextrose (YPD) agar plates containing Zeocin. After 2 days, starter seed cultures of the production strains were inoculated into 100 mL of Buffered Glycerol complex Medium (BMGY) using cells from these plates, and grown for 20-24 hours at 30° C. and 225-250 rpm. Seed cultures were scaled up by transferring suitable amounts into 2-4 L of BMMY medium in a baffled Fermenter. Fermentations were carried out at 30° C. and under 1100 rpm of agitation, supplied via flat-blade impellers, for 48-72 hours. After the initial batch-phase of fermentation, sterile-filtered Methanol was added as feed whenever the dissolved oxygen level in the culture dipped below 30%. Alternatively, feed was added every 3 hours at 0.5% v/v of the starting batch culture. The final fermentation broth was centrifuged at 7000×g for 30 mins at 4° C. to obtain the cell-free supernatant. The variant polypeptides having alpha-amylase activity were identified as follows: supernatant was assayed for protein of interest expression by either SDS-PAGE or capillary electrophoresis.

A parent enzyme was selected and is identified as the amino acid sequence of SEQ ID NO:1, and fragments were created. The enzyme fragments were tested for expression, activity, and melting temperature. The results are shown in the table below.

TABLE 1

| | | | pNP | |
|---|---|---|---|---|
| | | BCA Specific | activity | |
| | | Activity 37° C. | at 25° C. | |
| Patent | Expression | pH 7.5 | delta | |
| Sequence No.: | mg/ml | (umole/min/mg) | abs/mg | Tm ° C. |
| SEQ ID NO: 1/13 | 0.55 | 595.5 | 14000 | 38 C. |
| SEQ ID NO: 2/14 | 0.92 | 471.7 | 5975 | |
| SEQ ID NO: 3/15 | 0.74 | 365.8 | 3828 | |
| SEQ ID NO: 4/16 | 0.38 | 487.9 | 6042 | |
| SEQ ID NO: 5/17 | 0.06 | 337.7 | 3227 | |
| SEQ ID NO: 6/18 | 2.10 | 241.4 | 5585 | 42-44 C. |
| SEQ ID NO: 7/19 | 0.60 | 247.2 | 4913 | |
| SEQ ID NO: 8/20 | 0.91 | 276.3 | 5350 | |
| SEQ ID NO: 9/21 | 0.67 | 157.9 | 4913 | |
| SEQ ID NO: 10/22 | 6.00 | 431.0 | 2778 | 50 C. |

### Example 6: Variant Alpha-Amylase Enzymes

A parent enzyme was selected and is identified in this application as the amino acid sequence of SEQ ID NO:6, which encoded by the nucleic acid sequence of SEQ ID NO.:18. The parent enzyme was engineered in the lab to generate non-naturally occurring alpha amylase variant enzymes having improve characteristics of the enzyme. The improved characteristics, include thermostability, pH, enzyme activity, expression, protease stability, or any combination thereof. The variant polypeptide enzymes were created starting with the parent enzyme and evolving it using Gene Site Saturation Mutagenesis (GSSM) of the parent enzyme as described in at least U.S. Pat. Nos. 6,562,594, 6,171,820, and 6,764,835; Error Prone PCR; and/or Tailored Multi-site combinatorial analysis.

Variant polypeptides having amylase activity were generated and tested for Performance on Cloth having stains CFT-CS28 and EMPA161 at both 25C and 40C, and compared to SEQ ID NO:6. In addition, the variant polypeptides having amylase activity were tested for protease stability and the results show the residual activity after a 30 min. protease challenge with 0.005% Savinase™ protease at 40C compared to SEQ ID NO.:6. The variant polypeptides having amylase activity are listed in the Table below and the X indicates that the new variant polypeptides having amylase activity have better in performance on cloth, protease stability, or both performance on cloth and protease stability when compared to SEQ ID NO:1. In addition, the melting temperature (Tm) is provided along with the difference in (Tm) when compared to the amylase of SEQ ID No.:1. The results as described in the table below show the amylase variants have improvements in performance on cloth, protease stability, both cloth performance and protease stability; thermostability, or any combination thereof.

TABLE 2

| | | | Both Cloth | | delta Tm (° C.) |
|---|---|---|---|---|---|
| Amylase Variant | | | performance | | from |
| SEQ ID NO: 6 and | Performance | Protease | and protease | | SEQ ID |
| modifications | on Cloth | stability | stability | Tm ° C. | NO: 1 |
| S378R | x | | | 50 | 12 |
| A364V | x | | | | |
| A127T | | x | | 50 | 12 |
| T202E | | | x | 53 | 15 |
| L381A | x | | | | |
| L39A | x | | | 50 | 12 |
| T202P | x | | | | |
| L381T | x | | | | |
| A248P | x | | | | |
| N355R | | | x | 52 | 14 |
| S380K | x | | | 52 | 14 |
| S410N | | | x | 52 | 14 |
| A342V | | | x | 51 | 13 |
| S101P | | x | | | |
| A127L | | x | | 51 | 13 |
| A127S | | x | | 49 | 11 |
| T305S | x | | | 49 | 11 |
| K365Q | | | x | | |
| G384Y | x | | | | |
| S122N | | | x | 54 | 16 |
| S122P | | x | | 54 | 16 |
| L123V | | | x | 46 | 8 |
| S122L | | x | | | |
| M412L | x | | | | |
| SEQ ID NO: 6 | | | | 49 | 11 |
| (pPicZ) | | | | | |
| SEQ ID NO: 6 | | | | 44 | 6 |
| (produced in AOX) | | | | | |

The single point mutations were combined into various combinations to generate new amylase variants. The thermostability of the amylase variants was measured using a ThermoFluor assay and the amylase variants were compared to the thermostability of parent amylase of SEQ ID NO:1. In addition, the protease stability of the amylase variants was tested in presence of protease in MOPS Buffer pH 7,samples stored at 25 degrees C. and 40 degrees C., and the amylase activity was measured using the pNP assay. The results as described in the table below show the amylase variants have improvements in thermostability (Tm° C.), residual activity as a percentage of the original amylase activity before the protease stability challenge, or any combination thereof.

TABLE 3

| Amylase variants having | | | % Residual | % Residual |
|---|---|---|---|---|
| SEQ ID NO: 6 | | Delta | activity at 25° C. | activity at 40° C. |
| and modifications as | Average | Tm ° C. from | after | after |
| described below | Tm ° C. | SEQ ID NO: 1 | 0.5% protease | 0.1% protease |
| SEQ ID NO: 1 | 38.00 | | | |
| 202E---355R---342V--- | 45.00 | 7.00 | | 59.00 |
| 378R---127T | | | | |
| 202E---410N---342V--- | 45.30 | 7.30 | | 57.00 |
| 378R---127T | | | | |
| 355R---410N---342V--- | 43.50 | 5.50 | | 38.00 |
| 378R---127T | | | | |
| 202E---355R---410N---342V | 43.60 | 5.60 | | 1.00 |
| 202E---355R---410N---127T | 45.30 | 7.30 | | 52.00 |
| 202E---355R---342V---378R | 43.80 | 5.80 | | 0.00 |
| 202E---355R---378R---127T | 45.10 | 7.10 | | 53.00 |
| 202E---410N---342V---127T | 44.60 | 6.60 | | 47.00 |
| 355R---410N---342V---378R | 42.00 | 4.00 | | 1.00 |
| 355R---410N---342V---127T | 43.10 | 5.10 | | 37.00 |
| 355R---410N---378R---127T | 44.80 | 6.80 | | 51.00 |
| 355R---342V---378R---127T | 43.40 | 5.40 | | 33.00 |
| 410N---342V---378R---127T | 43.30 | 5.30 | | 41.00 |
| 202E---355R---342V | 43.30 | 5.30 | | 0.00 |
| 202E---355R---127T | 44.90 | 6.90 | | 64.00 |
| 202E---410N---342V | 43.40 | 5.40 | | 1.00 |
| 202E---410N---378R | 43.80 | 5.80 | | 0.00 |
| 202E---378R---127T | 45.00 | 7.00 | | 48.00 |
| 355R---410N---342V | 41.80 | 3.80 | | 0.00 |
| 355R---410N---127T | 43.80 | 5.80 | | 34.00 |
| 355R---342V---127T | 43.80 | 5.80 | | 38.00 |
| 355R---378R---127T | 43.90 | 5.90 | | 40.00 |
| 410N---342V---378R | 42.25 | 4.25 | | 1.00 |
| 410N---342V---127T | 43.90 | 5.90 | | 47.00 |
| 410N---378R---127T | 43.50 | 5.50 | | 47.00 |
| 342V---378R---127T | 43.70 | 5.70 | | 23.00 |
| 202E---355R | 43.60 | 5.60 | | 1.00 |
| 202E---410N | 43.40 | 5.40 | | 1.00 |
| 202E---127T | 45.00 | 7.00 | | 64.00 |
| 355R---127T | 43.60 | 5.60 | | 49.00 |
| 410N---342V | 42.00 | 4.00 | | 1.00 |
| 410N---378R | 41.80 | 3.80 | | 1.00 |
| 410N---127T | 43.60 | 5.60 | | 54.00 |
| 342V---378R | 41.75 | 3.75 | | 0.00 |
| 342V---127T | 43.40 | 5.40 | | 47.00 |
| 378R---127T | 43.25 | 5.25 | | 53.00 |
| 39A-127S | 45.30 | 7.30 | | 51.00 |
| 39A-122N | 46.40 | 8.40 | | 55.00 |
| 380K-127S | 43.70 | 5.70 | | 50.00 |
| 380K-122N | 45.90 | 7.90 | | 72.00 |
| 305S-127S | 44.00 | 6.00 | | 67.00 |
| 305S-122N | 45.50 | 7.50 | | 60.00 |
| 378R---S122N | 47.00 | 9.00 | 66.25 | 57.82 |
| 355R---410N---378R---S122N | 46.80 | 8.80 | 107.45 | 44.07 |
| 202E---355R---378R---S122N | 48.30 | 10.30 | 108.65 | 60.19 |
| 202E---410N---342V---S122N | 47.90 | 9.90 | 113.00 | 68.07 |
| 355R---S122N | 46.40 | 8.40 | 109.20 | 44.76 |
| 355R---342V---S122N | 45.80 | 7.80 | 106.59 | 37.90 |
| 410N---342V---378R---S122N | 46.80 | 8.80 | 109.03 | 40.32 |
| 355R---410N---S122N | 46.80 | 8.80 | 104.11 | 49.12 |
| 410N---S122N | 47.00 | 9.00 | 100.16 | 46.98 |
| 410N---342V---S122N | 46.80 | 8.80 | 107.62 | 41.01 |
| 202E---355R---S122N | 48.30 | 10.30 | 102.61 | 29.99 |
| 410N---378R---S122N | 47.30 | 9.30 | 100.25 | 22.45 |
| 355R---410N---342V--- | 46.60 | 8.60 | 104.17 | 47.24 |
| 378R---S122N | | | | |
| 202E---S122N | 48.10 | 10.10 | 105.01 | 63.35 |
| 202E---355R---342V--- | 48.00 | 10.00 | 102.25 | 63.27 |
| 378R---S122N | | | | |
| 202E---355R---410N---S122N | 47.90 | 9.90 | 112.97 | 61.25 |
| 342V---378R---S122N | 46.60 | 8.60 | 106.11 | 45.44 |
| 202E---410N---342V--- | 48.00 | 10.00 | 95.13 | 62.94 |
| 378R---S122N | | | | |
| 355R---410N---342V---S122N | 46.60 | 8.60 | 118.52 | 48.15 |
| 202E---378R---S122N | 48.40 | 10.40 | 102.42 | 58.48 |
| 355R---342V---378R---S122N | 47.00 | 9.00 | 115.34 | 42.64 |
| 342V---S122N | 47.00 | 9.00 | 114.18 | 58.28 |
| 355R---378R---S122N | 47.50 | 9.50 | 100.00 | 60.00 |
| 202E---355R---342V---S122N | 48.70 | 10.70 | 100.00 | 60.00 |
| 202E---410N---378R---S122N | 48.90 | 10.90 | 100.00 | 60.00 |
| 202E---410N---S122N | 49.00 | 11.00 | 100.00 | 60.00 |
| 355R---410N---378R | 43.60 | 5.60 | 40.00 | 0.00 |
| 202E---355R---410N---378R | 45.00 | 7.00 | 40.00 | 0.00 |
| 355R---342V---378R | 43.50 | 5.50 | 40.00 | 0.00 |
| 202E---410N---342V---378R | 44.80 | 6.80 | 40.00 | 0.00 |
| 202E---355R---378R | 45.00 | 7.00 | 40.00 | 0.00 |
| 202E---355R---410N | 45.10 | 7.10 | 40.00 | 0.00 |
| 202E---342V---378R---S122N | 49.00 | 11.00 | 100.00 | 60.00 |
| 202E---342V---S122N | 49.10 | 11.10 | 100.00 | 60.00 |
| 202E---355R---410N--- | 48.80 | 10.80 | 100.00 | 60.00 |
| 342V---378R---S122N | | | | |
| 202E---355R---410N--- | 49.10 | 11.10 | 100.00 | 60.00 |
| 378R---S122N | | | | |
| 202E---342V | 44.75 | 6.75 | 40.00 | 0.00 |
| 202E---378R | 45.10 | 7.10 | 40.00 | 0.00 |
| 202E---355R---410N--- | 44.50 | 6.50 | 40.00 | 0.00 |
| 342V---378R | | | | |
| 202E---355R---410N--- | 48.20 | 10.20 | 100.00 | 60.00 |
| 342V---S122N | | | | |
| 202E---342V---378R | 44.00 | 6.00 | 40.00 | 0.00 |
| 355R---410N | 43.50 | 5.50 | 40.00 | 0.00 |
| 355R---342V | 43.50 | 5.50 | 40.00 | 0.00 |
| 355R---378R | 43.40 | 5.40 | 40.00 | 0.00 |

The single point mutations were combined into various combinations to generate new amylase variants. The thermostability of the amylase variants was measured using a ThermoFluor assay and the amylase variants were compared to the thermostability of parent amylase of SEQ ID NOA. In addition, the protease stability of the amylase variants was tested in presence of protease (0.5% and 0.75% protease) in MOPS Buffer pH 7,samples stored at 37 degrees C., and the amylase activity was measured using the pNP assay. The results as described in the table below show the amylase variants have improvements in thermostability (Tm° C.), residual activity as a percentage of the original amylase activity before the protease stability challenge, or any combination thereof.

TABLE 4

| Amylase variants having | | | | % Residual |
|---|---|---|---|---|
| SEQ ID NO: 6 | Ave | Delta | % Residual activity | activity at 37° C. |
| and modifications | Tm | Tm ° C. from | at 37° C. after | after |
| as described below | ° C. | SEQ ID NO: 1 | 0.5% protease | 0.75% protease |
| 378R | 43.50 | 5.50 | 0.50 | −0.50 |
| 127T | 45.50 | 7.50 | 46.00 | 66.50 |
| 355R | 43.25 | 5.25 | 0.50 | 1.50 |
| S380 | 43.25 | 5.25 | 0.00 | 0.50 |
| 127S | 45.75 | 7.75 | 49.50 | 60.00 |
| 122N | 47.50 | 9.50 | 48.50 | 66.00 |
| 378R-355R-380K-122N-127T | | | | 71.00 |
| 378R-355R-380K-122P-127S | | | | 86.00 |
| 378R-355R-380K-122N-127S | | | | 24.00 |
| 378R-355R-380K-122P-127T | | | | 77.00 |
| 378R-380K-122N-127T | | | | 61.00 |
| 378R-380K-122P-127S | | | | 83.00 |
| 378R-380K-122N-127S | | | | 32.00 |
| 378R-355R-122N-127T | 46.63 | 8.63 | 70.50 | 77.00 |
| 378R-355R-122P-127S | 48.00 | 10.00 | 77.50 | 82.75 |
| 378R-355R-122N-127S | 46.25 | 8.25 | 45.25 | 58.50 |
| 378R-355R-122P-127T | 47.00 | 9.00 | 83.75 | 92.50 |
| 378R-122N-127T | 47.00 | 9.00 | 81.50 | 81.00 |
| 378R-122P-127S | 49.00 | 11.00 | 79.75 | 86.50 |
| 378R-122N-127S | 47.50 | 9.50 | 49.75 | 67.25 |
| 378R-122P-127T | 48.00 | 10.00 | 85.25 | 104.75 |
| 355R-122N-127T | 46.50 | 8.50 | 66.50 | 81.00 |
| 355R-122P-127S | 49.25 | 11.25 | 77.50 | 80.75 |
| 355R-122N-127S | 46.88 | 8.88 | 43.75 | 63.25 |
| 355R-122P-127T | 47.88 | 9.88 | 84.25 | 98.50 |
| 380K-122N-127T | 46.50 | 8.50 | 65.75 | 78.00 |
| 380K-122P-127S | 48.50 | 10.50 | 67.50 | 77.75 |
| 380K-122N-127S | 47.13 | 9.13 | 38.25 | 58.50 |
| 380K-122P-127T | 47.63 | 9.63 | 84.75 | 98.50 |
| 122N-138I | 44.00 | 6.00 | 12.50 | 12.25 |
| 122N-138N | 50.63 | 12.63 | 87.75 | 97.50 |
| 127T-138N | 48.38 | 10.38 | 93.50 | 95.75 |
| L138I | 19.00 | | | |
| L138N | 46.50 | 8.50 | 2.50 | 20.50 |
| SEQ ID NO: 1 | 38.00 | | | |
| SEQ ID NO: 6 | 44.00 | 6.00 | 0.00 | 0.00 |

The single point mutations were combined into various combinations to generate new amylase variants. The thermostability of the amylase variants was measured using a ThermoFluor assay and the amylase variants were compared to the thermostability of parent amylase of SEQ ID NO:1. In addition, the protease stability of the amylase variants was tested in presence of protease 0.75% protease) in MOPS Buffer pH 7, samples stored at 37 degrees C., and the amylase activity was measured using the pNP assay. The expression for the variant polypeptides having alpha-amylase activity were identified as follows: supernatant was assayed for protein of interest expression by either SODS-PAGE or capillary electrophoresis. The results as described in the table below show the amylase variants have improvements in thermostability (Tm° C.), average expression, residual activity as a percentage of the original amylase activity before the protease stability challenge, or any combination thereof.

TABLE 5

| Amylase variants having | | Delta Tm ° C. | Average | Avg. % Residual |
|---|---|---|---|---|
| SEQ ID NO: 6 and | Ave | from | Expression. | activity at 37° C. after |
| modifications as described below | Tm ° C. | SEQ ID NO: 1 | (mg/mL) | 0.75% protease |
| SEQ ID NO: 1 | 38.0 | | | |
| SEQ ID NO: 6 | 43.0 | 5.0 | 0.16 | 0 |
| 355R | 43.0 | 5.0 | 0.07 | 0 |
| 378R | 43.5 | 5.5 | 0.14 | 0 |
| S380 | 43.5 | 5.5 | 0.09 | 0 |
| 127T | 45.5 | 7.5 | 0.18 | 19 |
| S122P | 47.3 | 9.3 | 0.03 | 1 |
| S380K-S122P-A127T-L138M | 48.3 | 10.3 | 0.07 | 89 |
| S380K-S122P-A127T-L138N | 51.5 | 13.5 | 0.05 | 97 |
| S380K-S122P-A127T-L138P | 43.0 | 5.0 | 0.06 | |
| S380K-S122P-A127T-L138Q | 48.1 | 10.1 | 0.06 | 84 |
| S380K-S122P-A127T-L138K | — | | 0.05 | |
| S380K-S122P-A127T-L138H | 50.5 | 12.5 | 0.05 | 90 |
| S378R-S380K-S122P-A127T-L138M | 49.8 | 11.8 | 0.08 | 90 |
| S378R-S380K-S122P-A127T-L138N | 52.9 | 14.9 | 0.15 | 99 |
| S378R-S380K-S122P-A127T-L138P | 44.4 | 6.4 | 0.10 | |
| S378R-S380K-S122P-A127T-L138Q | 48.5 | 10.5 | 0.11 | 82 |
| S378R-S380K-S122P-A127T-L138K | 50.1 | 12.1 | 0.08 | 109 |
| S378R-S380K-S122P-A127T-L138H | 50.0 | 12.0 | 0.07 | 111 |
| S378R-355R-S122P-A127T-L138M | 49.2 | 11.2 | 0.08 | 81 |
| S378R-355R-S122P-A127T-L138N | 52.3 | 14.3 | 0.13 | 94 |
| S378R-355R-S122P-A127T-L138P | 43.3 | 5.3 | 0.08 | |
| S378R-355R-S122P-A127T-L138Q | 47.5 | 9.5 | 0.05 | 79 |
| S378R-355R-S122P-A127T-L138K | 49.8 | 11.8 | 0.08 | 90 |
| S378R-355R-S122P-A127T-L138H | 50.5 | 12.5 | 0.07 | 101 |
| 355R-S380K-S122P-A127T-L138M | 48.8 | 10.8 | 0.05 | 88 |
| 355R-S380K-S122P-A127T-L138N | 50.5 | 12.5 | 0.07 | 102 |
| 355R-S380K-S122N-A127T | 46.5 | 8.5 | 0.09 | 50 |
| 355R-S380K-S122P-A127S | 48.4 | 10.4 | 0.04 | 77 |
| 355R-S380K-S122N-A127S | 46.5 | 8.5 | 0.02 | 17 |
| 355R-S380K-S122P-A127T | 47.2 | 9.2 | 0.03 | 65 |
| S380K-S122P-A127T-L138N | 50.8 | 12.8 | 0.04 | |
| S378R-S380K-S122P-A127T | 48.8 | 10.8 | 0.09 | 80 |

### Example 7: Extended Evolution GSSM Library

The extended evolution started with the amino acid sequence of SEQ ID NO:10, which is encoded by the nucleic acid sequence of SEQ ID NO: 22, and then used Gene Site Saturation Mutagenesis (GSSM) to generate amylase variants. The thermostability of the amylase variants was measured using a ThermoFluor assay and the amylase variants were compared to the thermostability of parent amylase of SEQ ID NO:1. The expression for the variant polypeptides having alpha-amylase activity were identified as follows: supernatant was assayed for protein of interest expression by either SDS-PAGE or capillary electrophoresis. The residual activity was determined using the red starch assay. The results as described in the table below show the amylase variants have improvements in thermostability (Tm° C.), expression, residual activity as a percentage of the original parent amylase activity, or any combination thereof.

TABLE 6

| Amylase Variants | | delta Tm ° C. | | |
|---|---|---|---|---|
| SEQ ID NO: 10 | | fromSEQ | quant | residual |
| and modifications | Tm ° C. | ID NO: 1 | (mg/mL) | activity % |
| A136E | 48.5 | 10.5 | 0.033 | 31.61290323 |
| A216V-G241D | 49.5 | 11.5 | 0.033 | 42.47027741 |
| A36N+A71N+A136E+ | 48.5 | 10.5 | 0.035 | 28.49331713 |
| A148D+G209S | | | | |
| A71N+A136E+A148D+ | 48.5 | 10.5 | 0.030 | 31.00833965 |
| G209S+A409S | | | | |
| A71N+A136E+A148D+ | 48.5 | 10.5 | 0.030 | 44.17061611 |
| G209S+G354T | | | | |
| E253I | 48.5 | 10.5 | 0.027 | 30.87132141 |
| E253V | 49 | 11 | 0.061 | 44.48584203 |
| E253V-G361S | 49.5 | 11.5 | 0.096 | 55.26950925 |
| E253V-G363C | 49.5 | 11.5 | 0.055 | 37.86929275 |
| E253V-I309L-T376A | 50.5 | 12.5 | 0.055 | 65.11976048 |
| E253V-N331S | 50 | 12 | 0.056 | 44.65317919 |
| E253V-N340D | 50 | 12 | 0.064 | 63.98831264 |
| E253V-S322T | 50.5 | 12.5 | 0.066 | 56.89655172 |
| F285Y-L287V-N371Y- | 50.5 | 12.5 | 0.036 | 67.95302013 |
| L385Q-S410T | | | | |
| G103S-S108D | 50 | 12 | 0.046 | 49.57627119 |
| G105D | 50.5 | 12.5 | 0.037 | 61.55440415 |
| G105H | 50.5 | 12.5 | 0.021 | 60.44090631 |
| G105S | 50.5 | 12.5 | 0.020 | 56.96202532 |
| G105S-T127S | 50.5 | 12.5 | 0.055 | 71.76025918 |
| G105Y | 49.5 | 11.5 | 0.035 | 56.77252106 |
| G209E | 50 | 12 | | 53.42939481 |
| G213C | 48 | 10 | | 46.88102894 |
| G213D | 48.5 | 10.5 | 0.055 | 42.1625 |
| G213E | 48.5 | 10.5 | 0.057 | 56.0863 |
| G241D | 48.5 | 10.5 | 0.053 | 44.8238 |
| H119E+A120P+G121C+P122E+ | 47.5 | 9.5 | 0.089 | 34.844 |
| L123V+T124N+G135C | | | | |
| H119S+A120P+G121C+P122E+ | 48 | 10 | 0.053 | 44.0442 |
| L123V+T124N+G135C | | | | |
| I199W | 48 | 10 | 0.059 | 38.6921 |
| K221R-E253V | 0 | −38 | 0.000 | 360.8696 |
| K380R | 48 | 10 | 0.050 | 36.8502 |
| L123I | 48.5 | 10.5 | 0.053 | 38.804 |
| N103D | 48.5 | 10.5 | 0.055 | 46.1781 |
| N103D-G105S | 47 | 9 | 0.084 | 44.8224 |
| N130W | 47 | 9 | 0.051 | 40.4787 |
| N416E | 47 | 9 | 0.059 | 56.6826 |
| Q233L-E253V-T376A | 47.5 | 9.5 | 0.056 | 33.5933 |
| Q233R-E253V | 47.5 | 9.5 | 0.112 | 56.7076 |
| Q88H-L138M | 47 | 9 | 0.066 | 34.2313 |
| Q88H-S122D | 47 | 9 | | 31.71219187 |
| Q88H-S122D-L138M | 47 | 9 | | 35.0182704 |
| Q88H-S122E | 47.5 | 9.5 | | 33.4384858 |
| S101P | 47 | 9 | 0.070 | 37.60572544 |
| S101R-N103D | 47.5 | 9.5 | 0.062 | 28.17990162 |
| S106F | 47.5 | 9.5 | 0.074 | 39.71559167 |
| S178D | 50 | 12 | 0.074 | |
| S346G | 50 | 12 | 0.088 | |
| S93Q | 52.5 | 14.5 | 0.109 | |
| T202A | 51 | 13 | 0.060 | |
| T404I | 49.5 | 11.5 | 0.065 | |
| SEQ ID NO: 10/22 | 52 | 14 | 0.080 | |
| Y195F-E253V | 51 | 13 | 0.086 | |

### Example 8: Extended Evolution Synthetic Library

The extended evolution started with the amino acid sequence of SEQ ID NO:10, which is encoded by the nucleic acid sequence of SEQ ID NO:22, and the amylase variants were synthetic created. The thermostability of the amylase variants was measured using a ThermoFluor assay and the amylase variants were compared to the thermostability of parent amylase of SEQ ID NO:1. The amylase activity and % residual activity was determined using a red starch assay. The results as described in the table below show the amylase variants have improvements in thermostability (TmC), and residual activity as a percentage of the original parent amylase activity, or any combination thereof.

TABLE 7

| | | Change Tm ° C. | | |
|---|---|---|---|---|
| Amylase variants | | from SEQ ID | Red | residual |
| SEQ ID NO: 10 and modifications | Tm ° C. | NO: 1 | Starch | activity % |
| A36N-A136E+A148D+G209S | 49.50 | 11.50 | 0.77 | 46.70 |
| A36N-A71N+A136E+A148D | 49.00 | 11.00 | 0.87 | 46.00 |
| A36N-A71N+A136E+A148D+G209S+E253V | 53.00 | 15.00 | 0.82 | 51.20 |
| A36N-A71N+A136E+G209S | 49.50 | 11.50 | 0.63 | 41.80 |
| A36N-A71N+A148D+G209S | 49.00 | 11.00 | 1.25 | 67.80 |
| A36N-A71N+A148D+G209S+E253V | 53.00 | 15.00 | 0.65 | 51.30 |
| A36N-A71N+A148D+G209S+Q88H+L138M | 51.50 | 13.50 | 0.86 | 59.60 |
| H119E-A120P-G121C-P122E-L123V-T124N- | 51.50 | 13.50 | 0.69 | 55.80 |
| G135C-E253V | | | | |
| H119E-A120P-G121C-P122E-L123V-T124N- | 54.50 | 16.50 | 0.75 | 46.00 |
| G135C-E253V-Q88H | | | | |
| H119E-A120P-G121C-P122E-L123V-T124N- | 53.50 | 15.50 | 0.74 | 45.00 |
| G135C-E253V-Q88H-L138M | | | | |
| Q88H-L138M-E253V | 51.50 | 13.50 | 0.75 | 59.20 |
| Q88H-L138M-E253V-A36N-A71N-A136E- | 55.00 | 17.00 | 0.79 | 60.10 |
| A148D-G209S | | | | |
| Q88H-L18M-E253V-A36N-A71N-A136E- | 51.50 | 13.50 | 0.62 | 40.80 |
| A148D-G209S-K221R-N340D-S322T-S122D | | | | |
| Q88H-L138M-E253V-A36N-A71N-A148D- | 54.50 | 16.50 | 1.00 | 60.90 |
| G209S | | | | |
| Q88H-L138M-E253V-A36N-A71N-A148D- | 54.50 | 16.50 | 0.75 | 59.40 |
| G209S-K221R-N340D-S322T | | | | |
| Q88H-L138M-E253V-A71N-A136E-A148D- | 53.00 | 15.00 | 0.75 | 55.70 |
| G209S | | | | |
| Q88H-L138M-E253V-A71N-A148D-G209S | 52.50 | 14.50 | 0.65 | 56.60 |
| Q88H-L138M-E253V-G105S-T1275S | 55.00 | 17.00 | 0.96 | 50.00 |
| Q88H-L138M-E253V-G105S-T127S-A36N | 56.50 | 18.50 | 0.75 | 64.30 |
| Q88H-L138M-E253V-G105S-T127S-A36N- | 57.00 | 19.00 | 0.74 | 49.50 |
| A71N-A136E-A148D-G209S | | | | |
| Q88H-L138M-E253V-G105S-T127S-A36N- | 52.00 | 14.00 | 0.50 | 42.20 |
| A71N-A136E-A148D-G209S-K221R-N340D- | | | | |
| S322T-S122D | | | | |
| Q88H-L138M-E253V-G105S-T127S-A36N- | 57.00 | 19.00 | 0.70 | 52.60 |
| A71N-A148D-G209S | | | | |
| Q88H-L138M-E253V-G105S-T127S-A36N- | 57.00 | 19.00 | 1.30 | 114.70 |
| A71N-A148D-G2095-K221R-N340D-S322T | | | | |
| Q88H-L138M-E253V-G105S-T127S-I309L- | 55.00 | 17.00 | 0.96 | 58.90 |
| T376A | | | | |
| Q88H-L138M-E253V-G105S-T127S-I309L- | 57.00 | 19.00 | 0.57 | 43.10 |
| T376A-A36N-A71N-A136E-A148D-G2095 | | | | |
| Q88H-L138M-E253V-G105S-T127S-I309L- | 52.00 | 14.00 | 0.49 | 34.30 |
| T376A-A36N-A71N-A136E-A148D-G209S- | | | | |
| K221R-N340D-S322T-S122D | | | | |
| Q88H-L138M-E253V-G105S-T127S-I309L- | 56.50 | 18.50 | 1.66 | 110.20 |
| T376A-A36N-A71N-A148D-G209S | | | | |
| Q88H-L138M-E253V-G105S-T127S-I309L- | 56.50 | 18.50 | 0.72 | 49.30 |
| T376A-A36N-A71N-A148D-G209S-K221R- | | | | |
| N340D-S322T | | | | |
| Q88H-L138M-E253V-G105S-T127S-I309L- | 53.00 | 15.00 | 0.55 | 41.20 |
| T376A-K221R-N340D-S322T-S122D-G209E | | | | |
| Q88H-L138M-E253V-G105S-T1275-K221R- | 53.00 | 15.00 | 0.62 | 48.00 |
| N340D-S322T-S122D-G209E | | | | |
| Q88H-L138M-E253V-G105S-T127S-N331S- | 55.00 | 17.00 | 0.94 | 47.30 |
| G361S | | | | |
| Q88H-L138M-E253V-G105S-T127S-N331S- | 57.50 | 19.50 | 0.97 | 55.80 |
| G361S-A36N-A71N-A136E-A148D-G209S | | | | |
| Q88H-L138M-E253V-G105S-T127S-N331S- | 52.00 | 14.00 | 0.69 | 41.80 |
| G361S-A36N-A71N-A136E-A148D-G209S- | | | | |
| K221R-N340D-S322T-S122D | | | | |
| Q88H-L138M-E253V-G105S-T127S-N331S- | 57.00 | 19.00 | 0.71 | 50.50 |
| G361S-A36N-A71N-A148D-G209S | | | | |
| Q88H-L138M-E253V-G105S-T127S-N331S- | 57.00 | 19.00 | 0.74 | 50.60 |
| G361S-A36N-A71N-A148D-G209S-K221R- | | | | |
| N340D-5322T | | | | |
| Q88H-L138M-E253V-G105S-T127S-N331S- | 53.00 | 15.00 | 0.60 | 46.10 |
| G361S-K221R-N340D-S322T-S122D-G209E | | | | |
| Q88H-L138M-E253V-I309L-T376A | 51.50 | 13.50 | 0.62 | 44.90 |
| Q88H-L138M-E253V-I309L-T376A-A36N | 53.50 | 15.50 | 0.77 | 60.40 |
| Q88H-L138M-E253V-I309L-T376A-A36N- | 54.50 | 16.50 | 0.72 | 55.90 |
| A71N-A136E-A148D-G209S | | | | |
| Q88H-L138M-E253V-I309L-T376A-A36N- | 51.00 | 13.00 | 0.60 | 39.40 |
| A71N-A136E-A148D-G209S-K221R-N340D- | | | | |
| S322T-S122D | | | | |
| Q88H-L138M-E253V-I309L-T376A-A36N- | 54.50 | 16.50 | 0.69 | 55.30 |
| A71N-A148D-G2095 | | | | |
| Q88H-L138M-E253V-I309L-T376A-A36N- | 54.00 | 16.00 | 1.19 | 82.90 |
| A71N-A148D-G209S-K221R-N340D-S322T | | | | |
| Q88H-L138M-E253V-I309L-T376A-K221R- | 52.00 | 14.00 | 0.61 | 47.40 |
| N340D-S322T-S122D-G209E | | | | |
| Q88H-L138M-E253V-K221R-N340D-S322T- | 52.50 | 14.50 | 0.56 | 48.00 |
| S122D-G209E | | | | |
| Q88H-L138M-E253V-N331S-G361S | 51.50 | 13.50 | 0.92 | 58.90 |
| Q88H-L138M-E253V-N331S-G361S-A36N | 54.00 | 16.00 | 0.69 | 50.80 |
| Q88H-L138M-E253V-N331S-G361S-A36N- | 55.50 | 17.50 | 0.98 | 50.90 |
| A71N-A136E-A148D-G209S | | | | |
| Q88H-L138M-E253V-N331S-G361S-A36N- | 51.50 | 13.50 | 0.69 | 58.00 |
| A71N-A136E-A148D-G209S-K221R-N340D- | | | | |
| S322T-S122D | | | | |
| Q88H-L138M-E253V-N331S-G361S-A36N- | 55.00 | 17.00 | 1.26 | 64.70 |
| A71N-A148D-G209S | | | | |
| Q88H-L138M-E253V-N331S-G361S-A36N- | 54.50 | 16.50 | 0.78 | 52.50 |
| A71N-A148D-G209S-K221R-N340D-S322T | | | | |
| Q88H-L138M-E253V-N331S-G361S-K221R- | 52.50 | 14.50 | 0.60 | 58.10 |
| N340D-S322T-S122D-G209E | | | | |
| Q88H-L138M-G105S-T127S | 51.50 | 13.50 | 0.54 | 38.20 |
| Q88H-L138M-S101R-N103D | 50.00 | 12.00 | 0.63 | 34.30 |
| Q88H-L138M-S122D-E253V | 51.00 | 13.00 | 1.21 | 84.70 |
| Q88H-L138M-S122E-E253V | 51.00 | 13.00 | 1.05 | 54.50 |
| Q88H-S122D-E253V | 51.50 | 13.50 | 0.75 | 51.80 |

### Example 9: Extended Evolution

The extended evolution started with the amino acid sequence of SEQ ID NO:10, which is encoded by the nucleic acid sequence of SEQ ID NO: 22, and the amylase variants were synthetic created. The thermostability of the amylase variants was measured using a ThermoFluor assay and the amylase variants were compared to the thermostability of parent amylase of SEQ ID NO: 1. The amylase activity was determined using a red starch assay. The results as described in the table below show the amylase variants have improvements in thermostability (Tm° C.), and residual activity as a percentage of the original parent amylase activity, or any combination thereof.

TABLE 8

| Amylase Variants | | | Activity |
|---|---|---|---|
| SEQ ID NO: 10 and | | ΔTm ° C. | Remaining |
| modifications | Tm ° C. | SEQ ID NO: 1 | (%) |
| G105S-S108D---T127S--- | 53.5 | 15.5 | 79.0 |
| G209E---Q233R---N331S | | | |
| G105S-S108D---T127S--- | 53.5 | 15.5 | 92.6 |
| G209E---N331S | | | |
| G105S-S108D---G209E--- | 52.5 | 14.5 | 81.6 |
| Q233R---N331S | | | |
| G105S-S108D---G209E--- | 52.5 | 14.5 | 69.8 |
| N331S | | | |
| T127S---G209E---Q233R--- | 52.5 | 14.5 | 59.2 |
| N331S | | | |
| T127S---G209E---N331S | 52.5 | 14.5 | 76.7 |
| G209E---Q233R---N331S | 51.0 | 13.0 | 89.7 |
| G209E---Q233R | 51.5 | 13.5 | 68.0 |
| G209E---N331S | 51.5 | 13.5 | 79.0 |
| N103D---G105S-S108D--- | 52.5 | 14.5 | 109.9 |
| T127S---G209E---Q233R--- | | | |
| N331S | | | |
| N103D---G105S-S108D--- | 51.5 | 13.5 | 102.3 |
| T127S---G209E---Q233R | | | |
| N103D---G105S-S108D--- | 51.5 | 13.5 | 86.9 |
| G209E---Q233R---N331S | | | |
| N103D---G105S-S108D--- | 52.0 | 14.0 | 76.2 |
| G209E---N331S | | | |
| N103D---T127S---G209E--- | 53.5 | 15.5 | 99.3 |
| Q233R---N331S | | | |
| N103D---T127S---G209E--- | 53.5 | 15.5 | 91.8 |
| Q233R | | | |
| N103D---T127S---G209E--- | 53.5 | 15.5 | 89.4 |
| N331S | | | |
| N103D---G209E---Q233R--- | 52.0 | 14.0 | 69.8 |
| N331S | | | |
| N103D---G209E---Q233R | 52.5 | 14.5 | 52.3 |
| N103D---G209E---N331S | 52.5 | 14.5 | 81.5 |
| G105S-S108D---T127S--- | 53.0 | 15.0 | 95.6 |
| G209C---Q233R---N331S | | | |
| G105S-S108D---T127S--- | 53.5 | 15.5 | 73.4 |
| G209C---N331S | | | |
| G105S-S108D--G209C--- | 52.5 | 14.5 | 83.9 |
| N331S | | | |
| T127S---G209C---Q233R--- | 52.0 | 14.0 | 94.0 |
| N331S | | | |
| T127S---G209C---Q233R | 52.0 | 14.0 | 95.3 |
| G209C---Q233R---N331S | 50.5 | 12.5 | 70.1 |
| G209C---N331S | 51.0 | 13.0 | 73.4 |
| N103D---G105S-S108D--- | 52.5 | 14.5 | 74.9 |
| T127S---G209C---Q233R--- | | | |
| N331S | | | |
| N103D---G105S-S108D--- | 52.5 | 14.5 | 78.9 |
| T127S---G209C---Q233R | | | |
| N103D---G105S-S108D--- | 53.0 | 15.0 | 73.6 |
| T127S---G209C | | | |
| N103D---G105S-S108D--- | 51.5 | 13.5 | 81.9 |
| G209C---Q233R | | | |
| N103D---G105S-S108D--- | 51.5 | 13.5 | 80.6 |
| G209C---N331S | | | |
| N103D---T127S---G209C--- | 53.0 | 15.0 | 93.3 |
| Q233R---N331S | | | |
| N103D---T127S---G209C--- | 53.0 | 15.0 | 77.0 |
| N331S | | | |
| N103D---T127S---G209C | 53.5 | 15.5 | 102.1 |
| N103D---G209C---Q233R | 52.0 | 14.0 | 95.9 |
| G105S-S108D---T127S--- | 52.0 | 14.0 | 88.1 |
| Q233R---N331S | | | |
| G105S-S108D---T127S--- | 52.0 | 14.0 | 73.9 |
| Q233R | | | |
| G105S-S108D---T127S--- | 52.5 | 14.5 | 91.0 |
| N331S | | | |
| G105S-S108D---T127S | 52.5 | 14.5 | 100.8 |
| G105S-S108D---Q233R--- | 51.0 | 13.0 | 91.8 |
| N331S | | | |
| G105S-S108D---Q233R | 51.0 | 13.0 | 78.0 |
| G105S-S108D---N331S | 51.5 | 13.5 | 62.4 |
| T127S---Q233R---N331S | 50.5 | 12.5 | 92.4 |
| T127S---Q233R | 50.5 | 12.5 | 109.4 |
| T127S---N331S | 51.0 | 13.0 | 93.8 |
| T127S | 51.0 | 13.0 | 102.9 |
| Q233R---N331S | 50.0 | 12.0 | 77.4 |
| Q233R | 50.0 | 12.0 | 87.2 |
| N331S | 50.0 | 12.0 | 78.1 |
| N103D---G105S-S108D--- | 51.5 | 13.5 | 83.2 |
| T127S---Q233R---N331S | | | |
| N103D---G105S-S108D--- | 52.5 | 14.5 | 90.9 |
| T127S---N331S | | | |
| N103D---G105S-S108D--- | 51.5 | 13.5 | 115.0 |
| T127S | | | |
| N103D---G105S-S108D--- | 50.0 | 12.0 | 101.5 |
| Q233R---N331S | | | |
| N103D---G105S-S108D--- | 50.0 | 12.0 | 88.5 |
| Q233R | | | |
| N103D---G105S-S108D--- | 51.0 | 13.0 | 66.0 |
| N331S | | | |
| N103D---G105S-S108D | 51.0 | 13.0 | 59.3 |
| N103D---T127S---Q233R--- | 52.0 | 14.0 | 65.0 |
| N331S | | | |
| N103D---T127S---Q233R | 52.0 | 14.0 | 69.6 |
| N103D---T127S---N331S | 52.0 | 14.0 | 92.3 |
| N103D---T127S | 52.5 | 14.5 | 78.9 |
| N103D---Q233R---N331S | 51.0 | 13.0 | 93.6 |
| N103D---Q233R | 51.0 | 13.0 | 111.5 |
| N103D---N331S | 51.0 | 13.0 | 97.7 |
| N103D | 51.5 | 13.5 | 97.1 |
| I309L---G361S---G363C--- | 49.5 | 11.5 | 66.5 |
| T376A | | | |
| I309L---G361S---T376A | 49.5 | 11.5 | 82.3 |
| I309L---G361S | 49.5 | 11.5 | 90.2 |
| I309L---G363C---T376A | 49.5 | 11.5 | 78.9 |
| I309L---T376A | 49.5 | 11.5 | 79.0 |
| I309L | 51.0 | 13.0 | 69.0 |
| G361S---T376A | 50.0 | 12.0 | 73.3 |
| G363C | 50.0 | 12.0 | 84.5 |
| T376A | 50.5 | 12.5 | 80.0 |
| Q233L---I309L---G361S--- | 48.5 | 10.5 | 36.9 |
| G363C---T376A | | | |
| Q233L---I309L---G361S--- | 48.5 | 10.5 | 63.1 |
| T376A | | | |
| Q233L---I309L---G3615 | 48.5 | 10.5 | 80.8 |
| Q233L---I309L---G3630--- | 48.5 | 10.5 | 56.2 |
| T376A | | | |
| Q233L---I309L---G363C | 48.5 | 10.5 | 62.2 |
| Q233L---I309L---T376A | 48.5 | 10.5 | 81.1 |
| Q233L---I309L | 48.5 | 10.5 | 71.3 |
| Q233L---G361S---G363C--- | 49.0 | 11.0 | 40.6 |
| T376A | | | |
| Q233L---G361S---T376A | 49.0 | 11.0 | 81.0 |
| Q233L---G361S | 49.0 | 11.0 | 64.5 |
| Q233L---G363C---T376A | 49.0 | 11.0 | 71.6 |
| Q233L---T376A | 49.0 | 11.0 | 98.4 |
| Q233L | 49.0 | 11.0 | 89.0 |
| SEQ ID NO: 10 | 47.0 | 9.0 | 61.8 |
| E253V | 50.5 | 12.5 | 73.5 |

### Example 10: Extended Evolution

The extended evolution started with the amino acid sequence of SEQ ID NO:10, which is encoded by the nucleic acid sequence of SEQ ID NO: 22, and the amylase variants were synthetically created. The thermostability of the amylase variants was measured using a ThermoFluor assay and the amylase variants were compared tote thermostability of parent amylase of SEQ ID NO: 1. The amylase activity was determined using a red starch assay. The results as described in the table below show the amylase variants have improvements in thermostability (Tm° C.), and residual activity as a percentage of the original parent amylase activity, or any combination thereof.

TABLE 9

| | Thermofluor- | | |
|---|---|---|---|
| Amylase Variants | Melting | Delta Tm | |
| SEQ ID NO: 10 and | Temperature | ° C. SEQ ID | Activity |
| Modifications | ° C. | NO: 1 | Remaining |
| G105S-S108D---T127S---G209E---Q233R | 53.5 | 15.5 | 75.8 |
| G105S-S108D---T127S---G209E | 54.0 | 16.0 | 77.7 |
| G105S-S108D---G209E---Q233R | 52.5 | 14.5 | 62.9 |
| G105S-S108D---G209E | 53.0 | 15.0 | 73.9 |
| T127S---G209E---Q233R | 52.5 | 14.5 | 81.4 |
| T127S---G209E | 52.5 | 14.5 | 93.3 |
| G209E | 51.5 | 13.5 | 91.3 |
| N103D---G105S-S108D---T127S---G209E---N331S | 53.0 | 15.0 | 65.6 |
| N103D---G105S-S108D---T127S---G209E | 53.0 | 15.0 | 91.9 |
| N103D---G105S-S108D---G209E---Q233R | 51.5 | 13.5 | 80.5 |
| N103D---G105S-S108D---G209E | 52.0 | 14.0 | 69.2 |
| N103D---T127S---G209E | 53.5 | 15.5 | 63.8 |
| N103D---G209E | 52.5 | 14.5 | 70.6 |
| G105S-S108D---T127S---G209C---Q233R | 53.0 | 15.0 | 92.2 |
| G105S-S108D---T127S---G209C | 53.5 | 15.5 | 76.5 |
| G105S-S108D---G209C---Q233R---N331S | 52.0 | 14.0 | 81.5 |
| G105S-S108D---G209C---Q233R | 52.0 | 14.0 | 79.0 |
| G105S-S108D---G209C | 52.5 | 14.5 | 52.6 |
| T127S---G209C---N331S | 52.0 | 14.0 | 74.0 |
| T127S---G209C | 52.0 | 14.0 | 74.6 |
| G209C---Q233R | 50.5 | 12.5 | 64.7 |
| G209C | 51.0 | 13.0 | 71.2 |
| N103D---G105S-S108D---T127S---G209C---N331S | 53.0 | 15.0 | 66.8 |
| N103D---G105S-S108D---G209C---Q233R---N331S | 51.5 | 13.5 | 60.6 |
| N103D---G105S-S108D---G209C | 52.0 | 14.0 | 67.9 |
| N103D---T127S---G209C---Q233R | 53.0 | 15.0 | 66.0 |
| N103D---G209C---Q233R---N331S | 52.0 | 14.0 | 61.3 |
| N103D---G209C--N331S | 52.0 | 14.0 | 48.3 |
| N103D---G209C | 52.5 | 14.5 | 64.1 |
| G105S-S108D | 51.5 | 13.5 | 79.8 |
| N103D---G105S-S108D---T127S---Q233R | 51.5 | 13.5 | 70.3 |
| I309L---G361S---G363C | 49.5 | 11.5 | 55.6 |
| I309L---G363C | 49.5 | 11.5 | 60.6 |
| G361S---G363C---T376A | 50.0 | 12.0 | 49.8 |
| G361S---G363C | 49.5 | 11.5 | 70.9 |
| G361S | 50.0 | 12.0 | 56.0 |
| G363C---T376A | 50.0 | 12.0 | 64.1 |
| Q233L---1309L---G361S---G363C | 48.5 | 10.5 | 28.5 |
| Q233L---G361S---G363C | 49.0 | 11.0 | 31.9 |
| Q233L---G363C | 49.0 | 11.0 | 53.8 |
| SEQ ID NO: 10 | 46.5 | 8.5 | 56.3 |
| E253V | 50.0 | 12.0 | 77.4 |

### Example 11: Wash Performance on Starch Stains

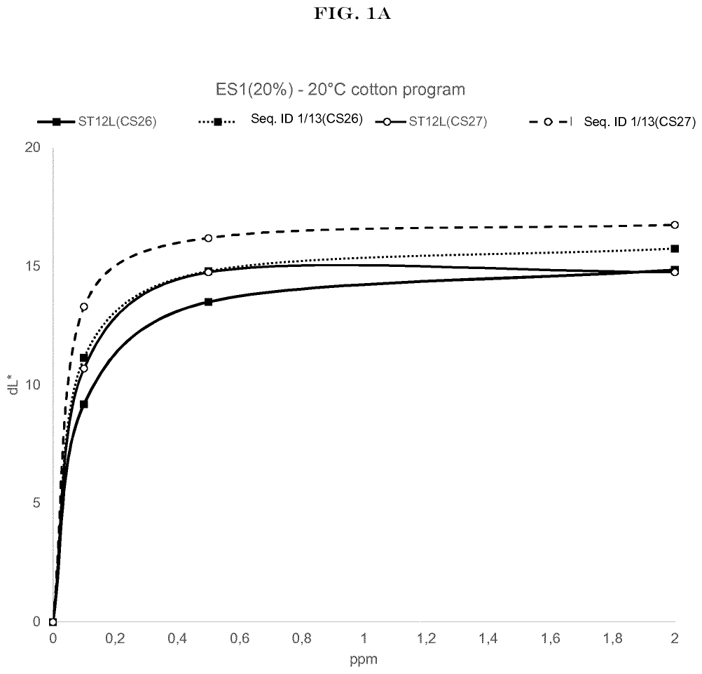

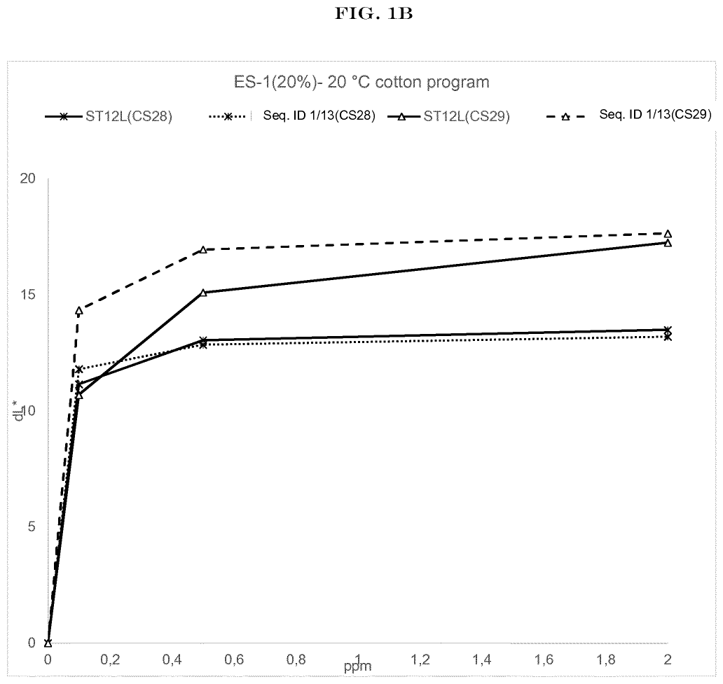

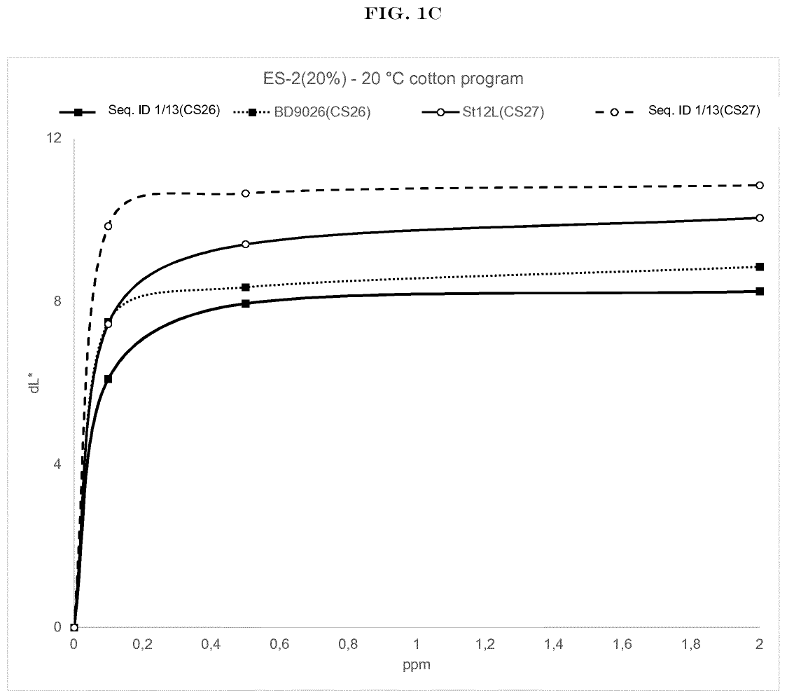

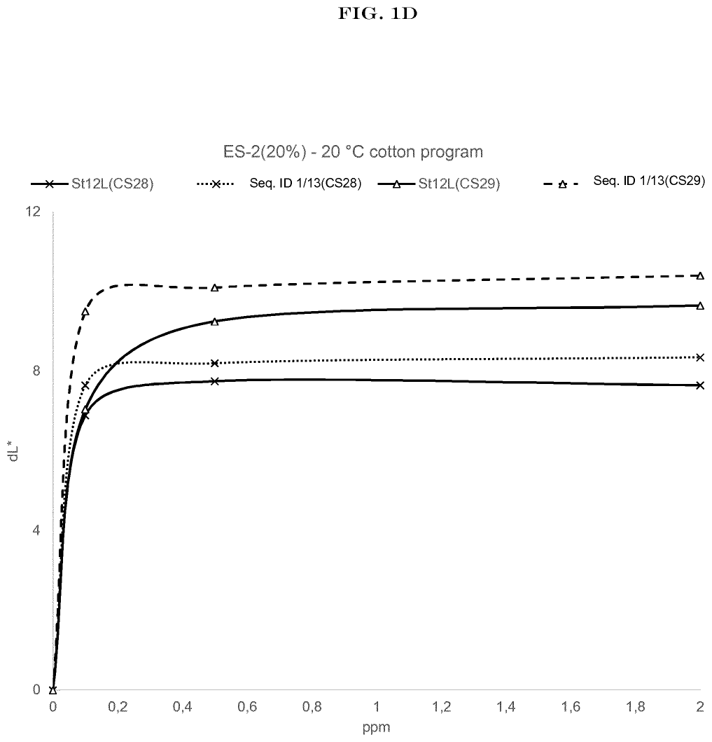

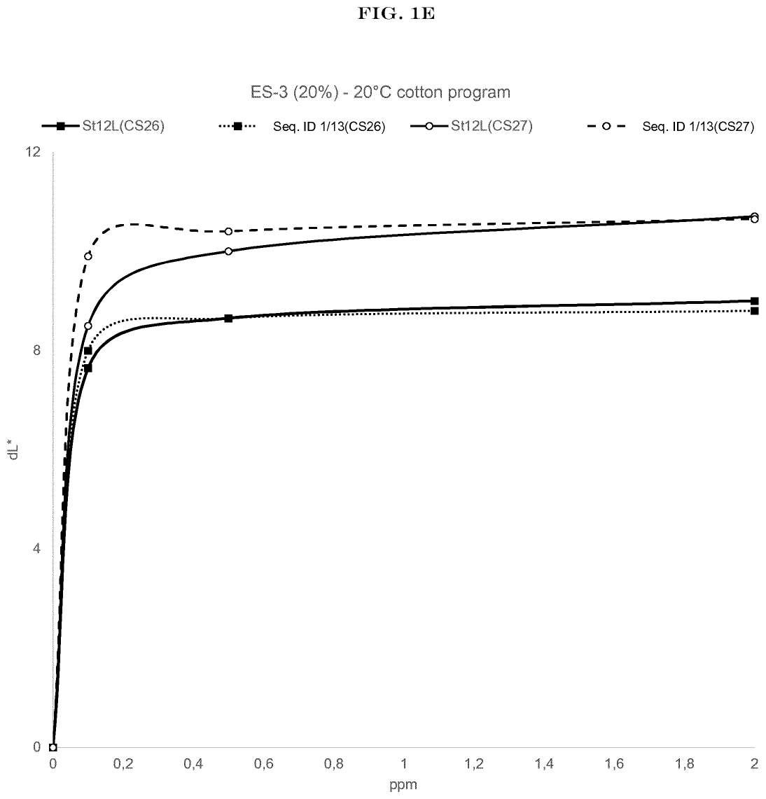

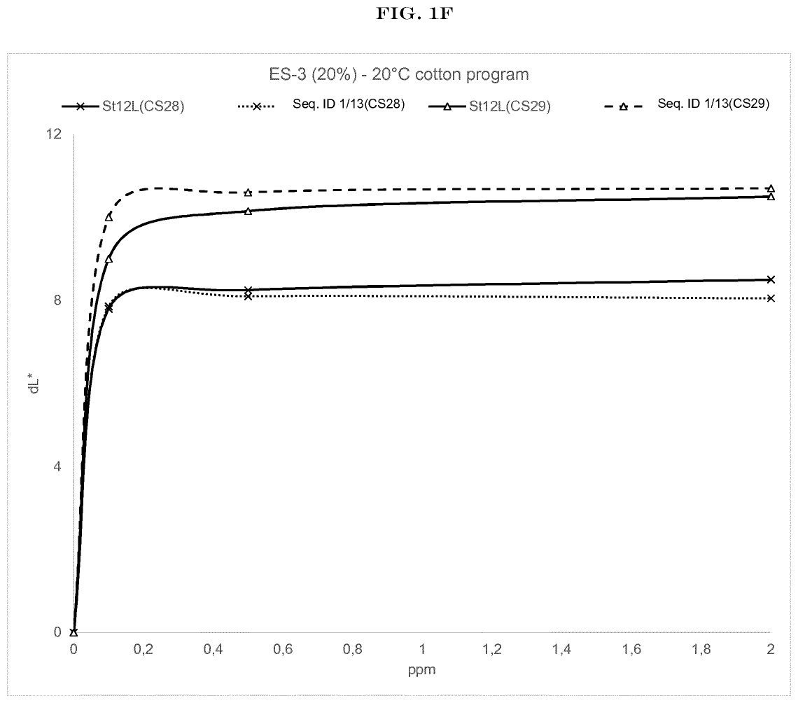

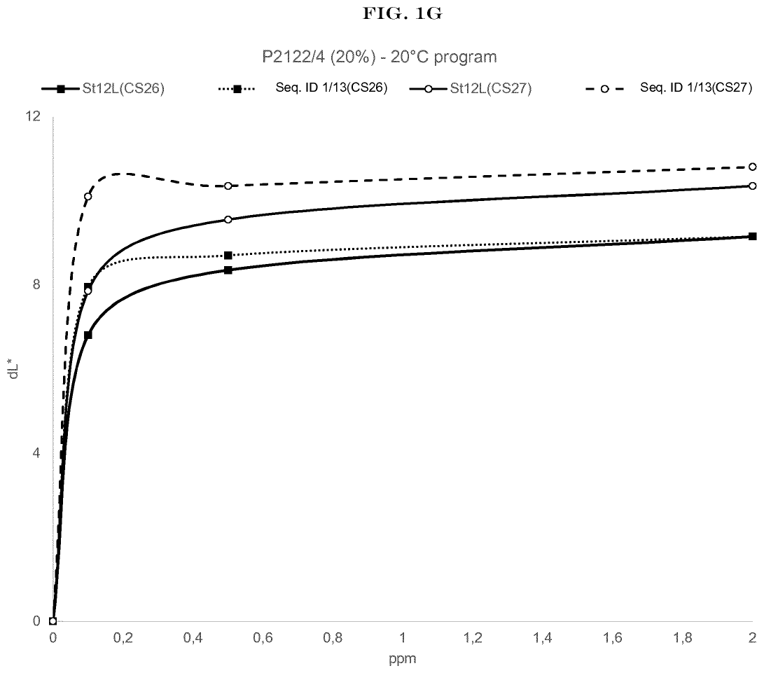

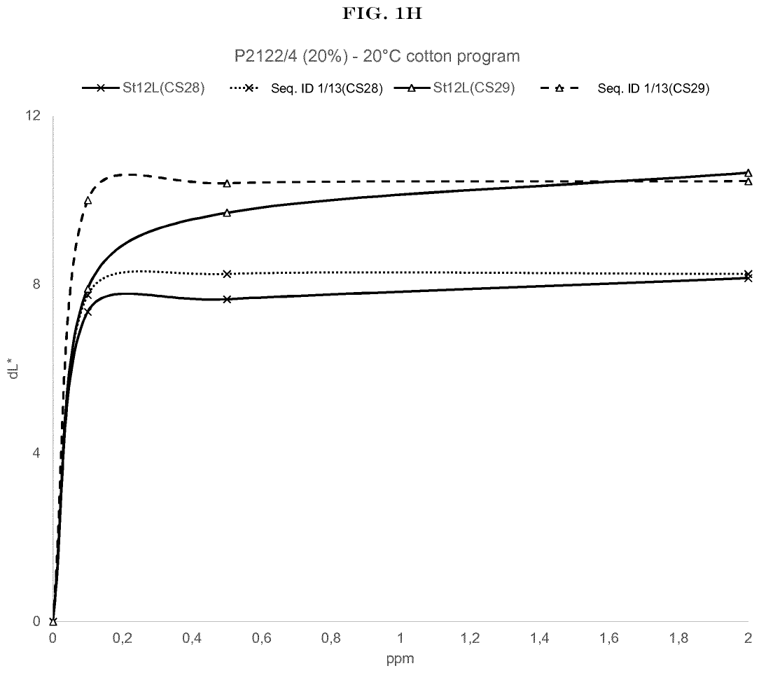

Wash performance of the amylase of SEQ ID NO:1 and Stainzyme 12L (ST12L) on various starch stains (CS-26, CS-27, CS-28, CS-29 obtained from Center for Testmaterials B.V.). The full scale test was performed with a European standard wash machine (front loader) using a standard cotton wash program at 20° C. The detergent formulation was dosed with 5 g/l to mimic European wash conditions. The results are shown in FIG. 1A and FIG. 1B, ES1 formulation; FIG. 1C and FIG. 1D ES2; FIG. 1 End FIG. 1F, ES-3; FIG. 1G and FIG. 1H, P2122/4 formulation.

### Example 12: Wash Performance on Cocoa Oatmeal Stain

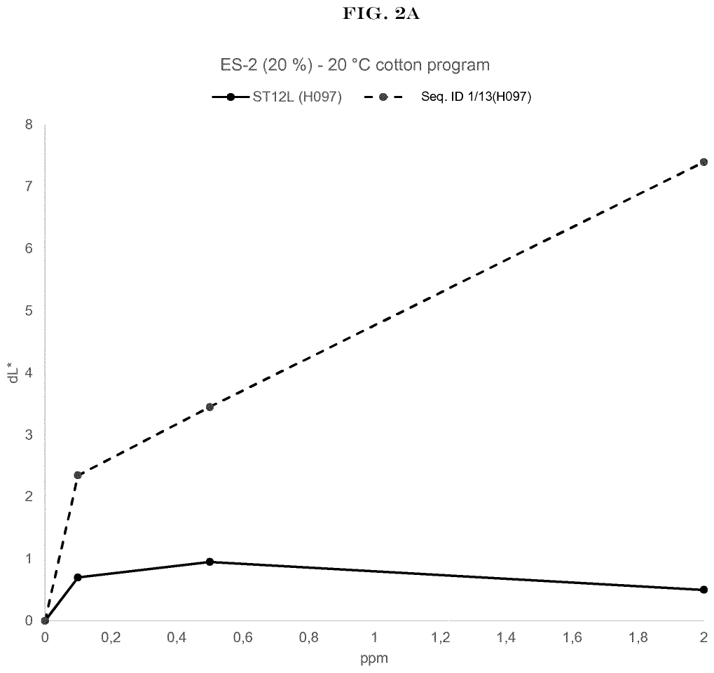

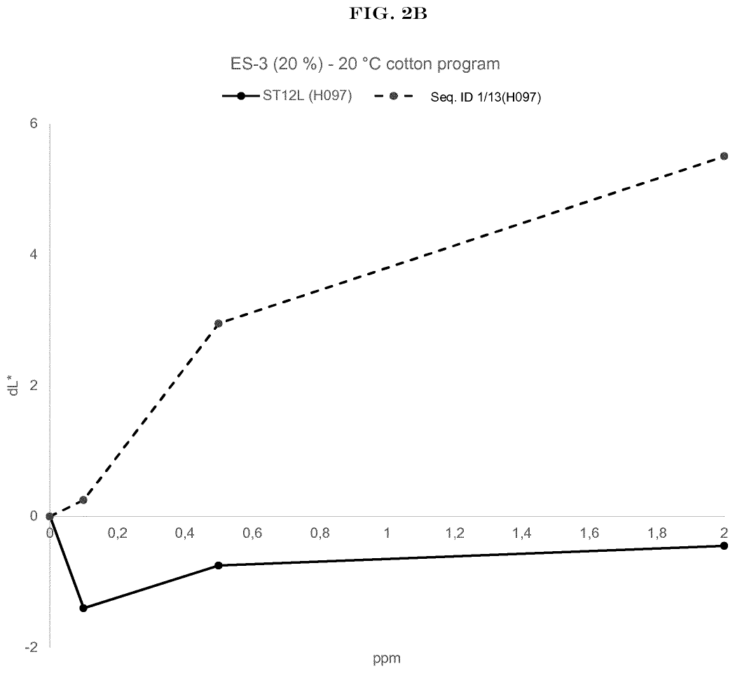

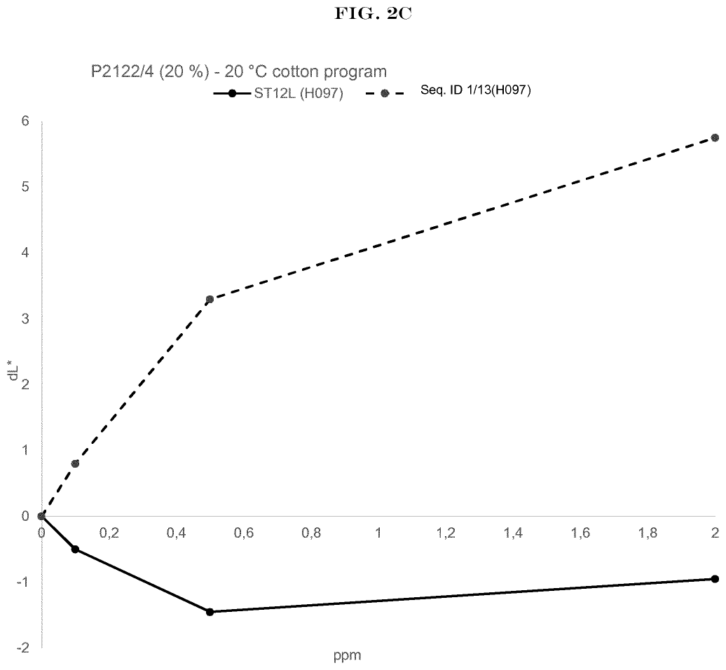

Wash performance of SEQ ID NO:1 and Stainzyme 12L (ST12L) on cocoa oatmeal stain (KC-H097 ex CFT). The full scale test was performed with a standard wash machine (front loader) using a standard cotton wash program at 20° C. The detergent formulation was dosed with 5 g/l to mimic European wash conditions. The results are shown in FIG. 2A, ES-2; FIG. 2B, ES-3; and FIG. 2C, P2122/4 formulation.

### Example 13: Wash Performance on Knitted Cotton

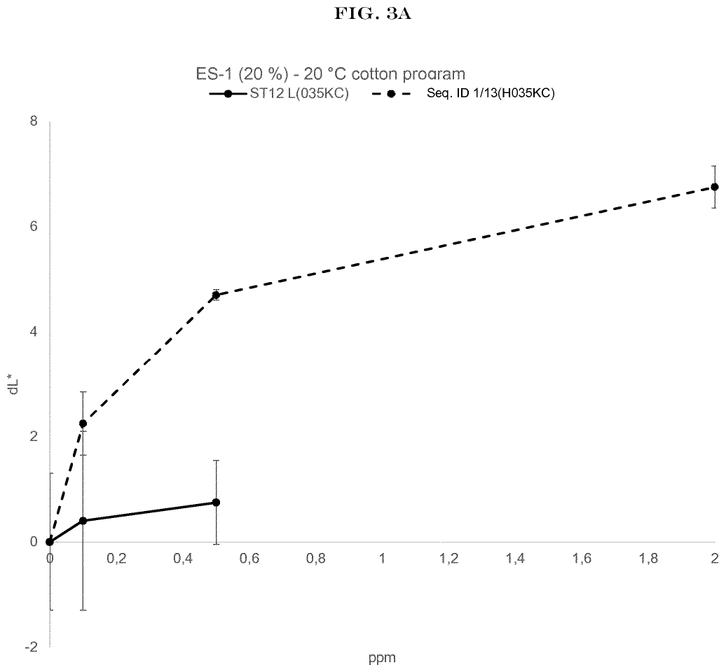

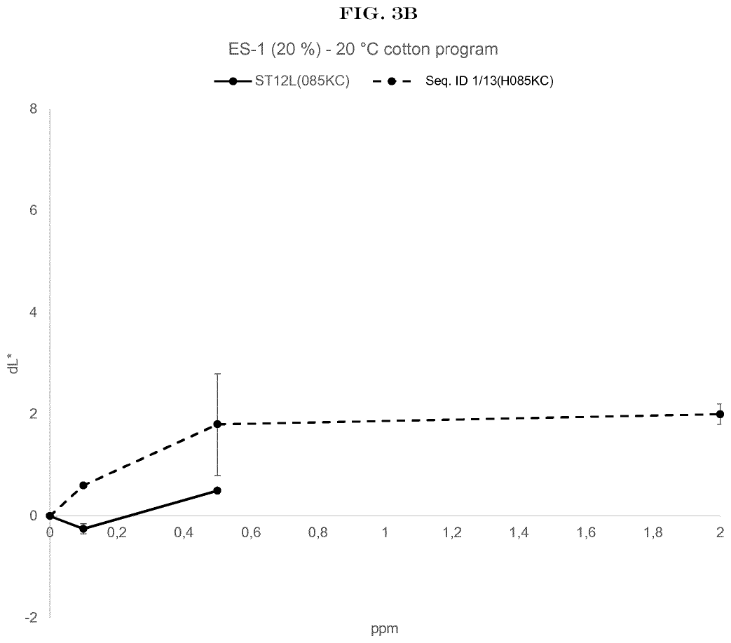

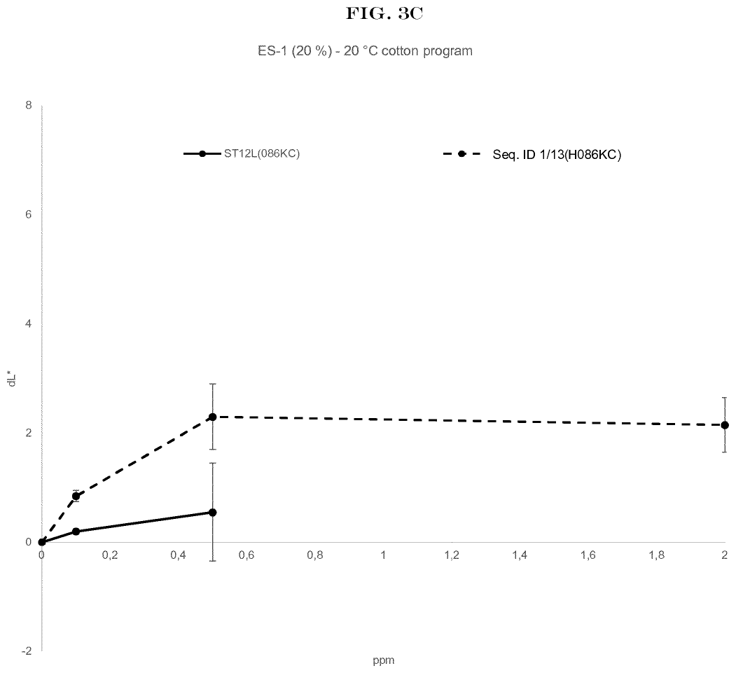

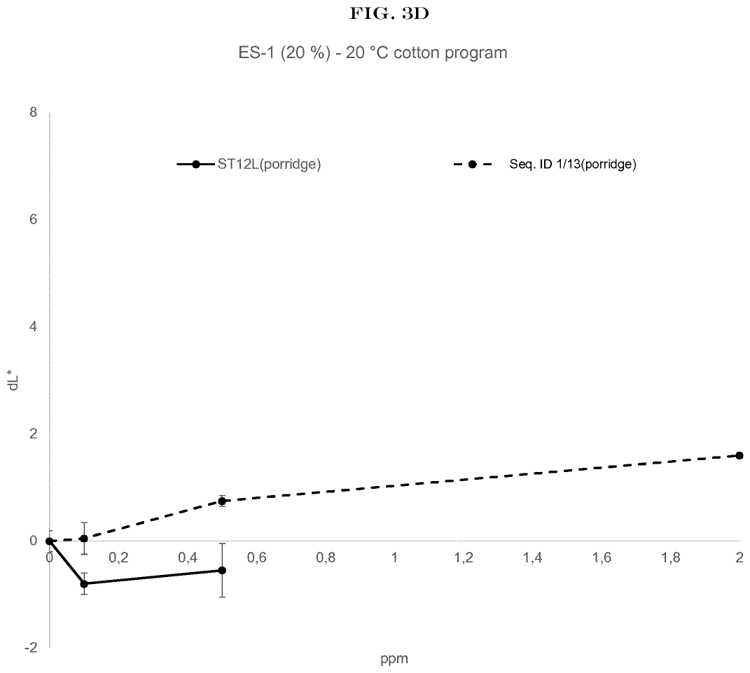

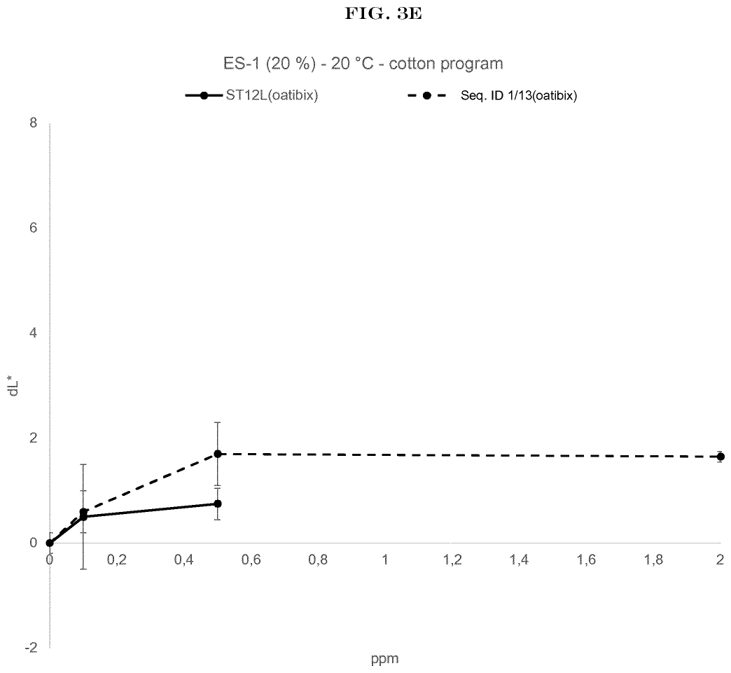

Wash performance of SEQ ID NO:1 and Stainzyme 12L (ST12L) on different fresh stains on knitted cotton from Warwick Equest Ltd. The full-scale test was performed with a standard wash machine (front loader) using a standard cotton wash program at 20° C. The detergent formulation ES-1 was dosed with 5 g/l to mimic European wash conditions. The results are shown in FIG. 3A, chocolate porridge oats (035); FIG. 3B, raspberry oats (085); FIG. 3C, golden syrup oats (086); FIG. 3D, porridge (−); and FIG. 3E, oatibix (−).

### Example 14: Wash Performance on Sum Over Different Starch Stains

Wash performance of the amylase variants in a non-bio commercial liquid laundry formulation on 11 different amylase sensitive stains on knitted cotton on basis of ddE. The amylase variants tested were: (A) SEQ ID NO:10 and S380K-S122P-A1271T; (B) BD53606 is SEQ ID NO:10 and S378R-355R-S122P-A127T-L138H; (C) BD53609 is SEQ ID NO:10 and S380K-S122P-A127T; (D) BD53629 is SEQ ID NO:10 and S378R-S380K-S122P-A127T-L138H; and, Stainzyme 12L.

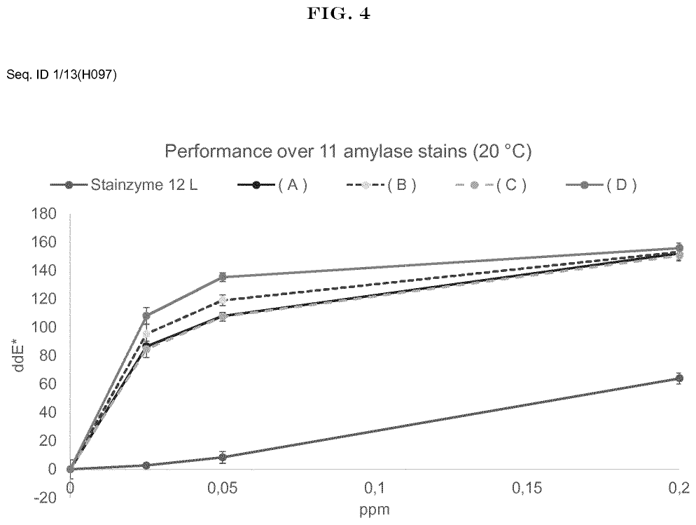

The full-scale test was performed with a standard wash machine (front loader) using a standard cotton wash program at 20° C. The detergent formulation was dosed with 5 g/l to mimic European wash conditions. The results are shown in FIG. 4, Sum over 11 different starch stains: C-S-26 (corn starch), C-S-27 (potato starch), C-S-28 (rice starch), C-S-29 (tapioca starch), C-S-126 (aged corn starch), C-S-127 (aged potato starch), C-S-128 (aged rice starch), C-S-129 (aged tapioca starch), EMPA160 (Chocolate Cream), EMPA161 (Corn starch), EMPA163 (Porridge) (C-S stains by Center for Testmaterials, NL, EMPA stains by Swissatest, CH).

### Example 15: 20180103 JB_Detergent Amylase Variant Table Normalized_Performance

The wash performance of amylase enzymes variants were tested in full scale test machines under the following conditions:
stain 1: EMPA161, 5 g/L ES 2 (20%), 20° C., FS, ddE at 0.2 ppm
stain 2: CFT C-S-26, 5 g/L ES 2 (20%), 20° C., FS, ddE at 0.2 ppm
stain 3: CFT C-S-27, 5 g/L ES 2 (20%), 20° C., FS, ddE at 0.2 ppm
stain 4: CFT C-S-28, 5 g/L ES 2 (20%), 20° C., FS, ddE at 0.2 ppm
stain 5: CFT C-S-29, 5 g/L ES 2 (20%), 20° C., FS, ddE at 0.2 ppm
stain 6: CFT C-S-126, 5 g/L ES 2 (20%), 20° C., FS, ddE at 0.2 ppm
stain 7: CFT C-S-127, 5 g/L ES 2 (20%), 20° C., FS, ddE at 0.2 ppm
stain 8: CFT C-S-128, 5 g/L ES 2 (20%), 20° C., FS, ddE at 0.2 ppm
stain 9: CFT C-S-129, 5 g/L ES 2 (20%), 20° C., FS, ddE at 0.2 ppm

The normalized results for these tests is shown below.

| | Stain | Stain | Stain | Stain | Stain | Stain | Stain | Stain | Stain |
|---|---|---|---|---|---|---|---|---|---|
| Enzyme | 1 | 2 | 3 | 4 | 5 | 6 | 7 | 8 | 9 |
| SEQ ID NO: 6/17 | 100 | 100 | 100 | 100 | 100 | 100 | 100 | 100 | 100 |
| SEQ ID NO: 6 and | 129 | 189 | 112 | 82 | 122 | 210 | 210 | 198 | 216 |
| S378R | | | | | | | | | |
| SEQ ID NO: 6 and | 177 | 188 | 131 | 86 | 128 | 223 | 311 | 205 | 242 |
| S380K | | | | | | | | | |
| SEQ ID NO: 6 and | 82 | 121 | 63 | 59 | 88 | 140 | 82 | 120 | 142 |
| N355R | | | | | | | | | |
| SEQ ID No: 6 and | 66 | 93 | 49 | 48 | 73 | 95 | 86 | 101 | 108 |
| A342V | | | | | | | | | |
| SEQ ID NO: 10 and | 297 | 223 | 197 | 130 | 146 | 216 | 472 | 265 | 343 |
| 355R-S380K-S122P- | | | | | | | | | |
| A127T-L138N | | | | | | | | | |
| SEQ ID NO: 10 and | 0 | 270 | 252 | 147 | 172 | 295 | 992 | 355 | 424 |
| S378R-355R-S122P- | | | | | | | | | |
| A127T-L138H | | | | | | | | | |
| SEQ ID NO: 10 | 241 | 193 | 162 | 134 | 133 | 195 | 218 | 205 | 287 |
| and S380K-S122P- | | | | | | | | | |
| A127T-L138H | | | | | | | | | |
| SEQ ID NO: 10 | 375 | 267 | 262 | 148 | 173 | 301 | 1160 | 372 | 448 |
| and S378R-S380K- | | | | | | | | | |
| S122P-A127T-L138H | | | | | | | | | |

The wash performance of amylase enzyme variants were tested in full scale test machines under the following conditions:
stain 10: CFT C-S-28, 5 g/L ES (20%), 20° C., LOM, ddL at 0.2 ppm
stain 11: EMPA161, 5 g/L ES 2(20%), 20° C., LOM, ddL at 0.2 ppm
stain 12: CFT C-S-28, 5 g/L ES2(20%), 40° C., LOM, ddL at 0.8 ppm
stain 13: EMPA161, 5 g/L ES 2(20%), 40° C., LOM, ddL at 0.8 ppm

The results are shown in the table below.

| Enzyme | Stain 10 | Stain 11 | Stain 12 | Stain 13 |
|---|---|---|---|---|
| SEQ ID NO: 6/17 | 100 | 100 | 100 | 100 |
| SEQ ID NO: 6 and A127T | 71 | 98 | | |
| SEQ ID NO: 6 and N355R | 101 | 96 | −32 | 90 |
| SEQ ID NO: 6 and A127L | 59 | 62 | | |
| SEQ ID NO: 6 and 5378R | 140 | 153 | 225 | 269 |
| SEQ ID NO: 6 and L39A | 100 | 127 | | |
| SEQ ID NO: 6 and S380K | 108 | 138 | 79 | 120 |
| SEQ ID NO: 6 and S380K | 145 | 174 | 199 | 258 |
| SEQ ID NO: 6 and A1275 | 51 | 39 | | |
| SEQ ID NO: 6 and T3055 | 130 | 143 | 162 | 137 |

1. A polypeptide having amylase activity comprising an amino acid sequence that is at least 80% identical, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the full length amino acid sequence of SEQ ID NO:2, SEQ ID NO:3, SEQ ID NO:4, SEQ ID NO:5, SEQ ID NO:6, SEQ ID NO:7, SEQ ID NO:8, SEQ ID NO:9, or SEQ ID NO:10.

2. The polypeptide of claim 1, comprising one or more amino acid residue insertions, deletions, substitutions, or any combinations thereof to the amino acid sequence of SEQ ID NO:2, SEQ ID NO:3, SEQ ID NO:4, SEQ ID NO:5, SEQ ID NO:6, SEQ ID NO:7, SEQ ID NO:8, SEQ ID NO:9, or SEQ ID NO:10.

3. The polypeptide of claim 2, wherein the one or more amino acid residue insertions, deletions, substitutions, or any combinations thereof is at the amino acid residue position number selected from the group consisting of: 36, 39, 67, 71, 88, 93, 101, 103, 105, 106, 108, 119, 120, 121, 122, 123, 124, 127, 130, 135, 136, 138, 148, 178, 195, 199, 202, 209, 213, 216, 221, 233, 241, 248, 253, 285, 287, 305, 309, 322, 331, 340, 342, 344, 346, 354, 355, 361, 363, 364, 365, 371, 376, 378, 380, 381, 384, 385, 404, 410, 412, and 416 of the amino acid sequence of SEQ ID NO:2, SEQ ID NO:3, SEQ ID NO:4, SEQ ID NO:5, SEQ ID NO:6, SEQ ID NO:7, SEQ ID NO:8, SEQ ID NO:9, or SEQ ID NO:10.

4. A polypeptide having amylase activity comprising an amino acid sequence that is at least 80% identical, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the full length amino acid sequence of SEQ ID NO:1, wherein the polypeptide comprises one or more amino acid residue insertion, deletion, or substitution or combinations thereof at the amino acid residue position number selected from the group consisting of: 36, 39, 67, 71, 88, 93, 101, 103, 105, 106, 108, 119, 120, 121, 122, 123, 124, 127, 130, 135, 136, 138, 148, 178, 195, 199, 202, 209, 213, 216, 221, 233, 241, 248, 253, 285, 287, 305, 309, 322, 331, 340, 342, 344, 346, 354, 355, 361, 363, 364, 365, 371, 376, 378, 380, 381, 384, 385, 404, 410, 412, and 416 of the amino acid sequence of SEQ ID NO: 1.

5. The polypeptide of claim 2, wherein the one or more amino acid substitution is a substitution selected from the group consisting of: A36N, L39A, L39R, K67R, A71N, Q88H, S93Q, S101P, S101R, N103D, G105S, G105D, G105H, G105Y, S106F, S108D, H₁₁₉E, H₁₁₉S, A120P, G121C, S122E, S122D, S122L, S122N, S122P, L123V, L1231, T124N, A127S, A127T, A127L, N130W, G135C, A136E, L1381, L138N, L138M, L138P, L138Q, L138K, L138H, A148D, S178D, Y195F, 1199W, T202E, T202P, T202A, G209S, G209E, G209C, G213D, G213E, G213C, A216V, K221R, Q233R, Q233L, G241D, A248P, E253C, E253V, E2531, F285Y, L287V, T305S, 1309L, S322T, N331S, N340D, A342V, G344H, S346G, G354T, N355R, G361S, G363C, A364V, K365A, K365Q, N371Y, T376A, S378R, S380K, S380R, L381A, L381T, G384Q, G384Y, L385Q, T4041, S410N, S41T, M412L, and N416E of the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, or SEQ ID NO:10.

6. A polypeptide comprising an amino acid sequence that is at least 80% identical, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the full length amino acid sequence as set forth in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, or SEQ ID NO:10; and one or more amino acid substitution, wherein the one or more amino acid substitution is selected from the group consisting of: A36N, L39A, L39R, K67R, A71N, Q88H, S93Q, S101P, S101R, N103D, G105S, G105D, G105H, G105Y, S106F, S108D, H₁₁₉E, H₁₁₉S, A120P, G121C, S122E, S122D, S122L, S122N, S122P, L123V, L1231, T124N, A127S, A127T, A127L, N130W, G135C, A136E, L1381, L138N, L138M, L138P, L138Q, L138K, L138H, A148D, S178D, Y195F, 1199W, T202E, T202P, T202A, G209S, G209E, G209C, G213D, G213E, G213C, A216V, K221R, Q233R, Q233L, G241D, A248P, E253C, E253V, E2531, F285Y, L287V, T305S, 1309L, S322T, N331S, N340D, A342V, G344H, S346G, G354T, N355R, G361 S, G363C, A364V, K365A, K365Q, N371 Y, T376A, S378R, S380K, S380R, L381A, L381T, G384Q, G384Y, L385Q, T4041, S410N, S41T, M412L, and N416E, of the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, or SEQ ID NO:10.

7. A polypeptide having amylase activity, wherein the polypeptide is an amino acid sequence that is at least 80% identical, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the full length amino acid sequence as set forth in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, or SEQ ID NO:10, wherein the poly-peptide has a combination of amino acid modifications to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, or SEQ ID NO:10; and the combination is selected from the group consisting of any mutations listed in Table 2 to Table 9.

8. A polypeptide having amylase activity, wherein the polypeptide is an amino acid sequence that is at least 80% identical, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the full length amino acid sequence as set forth in SEQ ID NO:2, SEQ ID NO:3, SEQ ID NO:4, SEQ ID NO:5, SEQ ID NO:6, SEQ ID NO:7, SEQ ID NO:8, SEQ ID NO:9, or SEQ ID NO:10, and the polypeptide has an in-crease in enzyme activity, thermostability, pH-stability, protease stability, or any combination thereof when compared to the amylase of SEQ ID NO:1.

9. A polypeptide having amylase activity, wherein the polypeptide comprises an amino acid sequence that is at least 80% identical, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the full length amino acid sequence as set forth in SEQ ID NO:1, wherein the polypeptide comprises one or more amino acid residue insertion, deletion, or substitution or combinations thereof at the amino acid residue position number selected from the group consisting of: 36, 39, 67, 71, 88, 93, 101, 103, 105, 106, 108, 119, 120, 121, 122, 123, 124, 127, 130, 135, 136, 138, 148, 178, 195, 199, 202, 209, 213, 216, 221, 233, 241, 248, 253, 285, 287, 305, 309, 322, 331, 340, 342, 344, 346, 354, 355, 361, 363, 364, 365, 371, 376, 378, 380, 381, 384, 385, 404, 410, 412, and 416 of the amino acid sequence of SEQ ID NO: 1, and the polypeptide has an increase in enzyme activity, thermostability, pH-stability, protease stability, or any combination thereof when compared to the amylase of SEQ ID NO:1.

10. The polypeptide of claim 1, wherein the polypeptide is a fragment of the full length amino acid sequence and the fragment has amylase activity.

11. A polypeptide comprising a hybrid of at least one polypeptide of claim 1, and a second polypeptide having an activity selected from the group consisting of amylase activity, protease activity, lipase activity, cellulase activity, laccase activity, pectinase activity, and nuclease activity, wherein the hybrid also has amylase activity.

12. A composition comprising the polypeptide of claim 1.

13. The composition of claim 12, further comprising a second enzyme selected from the group consisting of: a second amylase, a lipase, a protease, a cellulase, a laccase, a pectinase, a nuclease, and any combination thereof.

14. A polypeptide having amylase activity, which is encoded by a polynucleotide having a nucleic acid sequence at least 80% identical, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the full length polynucleotide sequence of SEQ ID NO:13, SEQ ID NO:14, SEQ ID NO:15, SEQ ID NO:16, SEQ ID NO:17, SEQ ID NO:18, SEQ ID NO:19, SEQ ID NQ:20, SEQ ID NO:21, or SEQ ID NO:22.

15. A polypeptide having amylase activity, which is encoded by a polynucleotide having a nucleic acid sequence at least 80% identical, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the full length polynucleotide sequence of SEQ ID NO: 13, wherein the polypeptide comprises one or more amino acid residue insertion, deletion, or substitution or combinations thereof at the amino acid residue position number selected from the group consisting of: 36, 39, 67, 71, 88, 93, 101, 103, 105, 106, 108, 119, 120, 121, 122, 123, 124, 127, 130, 135, 136, 138, 148, 178, 195, 199, 202, 209, 213, 216, 221, 233, 241, 248, 253, 285, 287, 305, 309, 322, 331, 340, 342, 344, 346, 354, 355, 361, 363, 364, 365, 371, 376, 378, 380, 381, 384, 385, 404, 410, 412, and 416 of the amino acid sequence of SEQ ID NO: 1.

16. A method of making the polypeptide of claim 1, comprising: providing a nucleic acid sequence, transforming the nucleic acid sequence into an expression host, cultivating the expression host to produce the polypeptide, and purifying the polypeptide.

17. The method of claim 16, wherein the expression host is selected from the group consisting of: a bacterial expression system, a yeast expression system, a fungal expression system, and a synthetic expression system.

18. The method of claim 17, wherein the bacterial expression system is selected from an E. coli, a Bacillus, a Pseudomonas, and a Streptomyces, wherein the yeast expression sys-tem is selected from a Candida, a Pichia, a Saccharomyces, a Schizosaccharomyces or, wherein the fungal expression system is selected from a Penicillium, an Aspergillus, a Fusarium, a Myceliopthora, a Themothelomyces, a Rhizomucor, a Rhizopus, a Thermomyces, and a Trichoderma.

19. A method of preparing a dough or a baked product prepared from the dough, the method comprising adding the polypeptide of claim 1, to the dough and baking it.

20. (canceled)
